SEQUENCE LISTING (24 sequences)

SEQ ID NO: 1 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 2 - protein, 365 aa, Homo sapiens [sequence omitted]

SEQ ID NO: 3 - protein, 158 aa, Homo sapiens
VGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLP

SEQ ID NO: 4 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 5 - protein, 497 aa, Synthetic construct
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVEDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVEFMPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAIT

SEQ ID NO: 6 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 7 - DNA, 3987 bp, Homo sapiens
  source 1..3987: mol_type = other DNA
cttcctggactggggatcccggctaaatatagctgtttctgtcttacaacacaggctccagtatataaatcaggcaaattccccatttgagcatgaacctctgaaaactgccggcatctgaggtttcctccaaggccctctgaagtgcagcccataatgaaggtcttggcggcaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctagcaggaggtcttgaagtgtgctgtgaaccgagggatctcaggagttgggtccagatgtgggggcctgtccaagggtggctggggcccagggcatcgctaaacccaaatgggggctgctggcagaccccgagggtgcctggccagtccactccactctgggctgggctgtgatgaagctgagcagagtggaaacttccatagggagggagctagaagaaggtgccccttcctctgggagattgtggactggggagcgtgggctggacttctgcctctacttgtccctttggccccttgctcactttgtgcagtgaacaaactacacaagtcatctacaagagccctgaccacagggtgagacagcagggcccaggggagtggaccagcccccagcaaattatcaccatctgtgcctttgctgccccttaggttgggacttaggtgggccagaggggctaggatcccaaaggactccttgtcccctagaagtttgatgagtggaagatagagaggggcctctgggatggaaggctgtcttcttttgaggatgatcagagaacttgggcataggaacaatctggcagaagtttccagaaggaggtcacttggcattcaggctcttggggaggcagagaagccaccttcaggcctgggaaggaagacactgggaggaggagaggcctggaaagctttggtaggttcttcgttctcttccccgtgatcttccctgcagcctgggatggccagggtctgatggctggacctgcagcaggggtttgtggaggtgggtagggcaggggcaggttgctaagtcaggtgcagaggttctgagggacccaggctcttcctctgggtaaaggtctgtaagaaggggctggggtagctcagagtagcagctcacatctgaggccctgggaggccttgtgaggtcacacagaggtacttgagggggactggaggccgtctctggtccccagggcaagggaacagcagaacttagggtcagggtctcagggaaccctgagctccaagcgtgctgtgcgtctgacctggcatgatttctatttattatgatatcctatttatattaacttattggtgctttcagtggccaagttaattcccctttccctggtccctactcaacaaaatatgatgatggctcccgacacaagcgccagggccagggcttagcagggcctggtctggaagtcgacaatgttacaagtggaataagccttacgggtgaagctcagagaagggtcggatctgagagaatggggaggcctgagtgggagtggggggccttgctccacccccccccatcccctactgtgacttgctttagggtgtcagggtccaggctgcaggggctgggccaatttgtggagaggccgggtgcctttctgtcttgattccagggggctggttcacactgttcttgggcgccccagcattgtgttgtgaggcgcactgttcctggcagatattgtgccccctggagcagtgggcaagacagtccttgtggcccaccctgtccttgtttctgtgtccccatgctgcctctgaaatagcgccctggaacaaccctgcccctgcacccagcatgctccgacacagcagggaagctcctcctgtggcccggacacccatagacggtgcggggggcctggctgggccagaccccaggaaggtggggtagactggggggatcagctgcccattgctcccaagaggaggagagggaggctgcagatgcctgggactcagaccaggaagctgtgggccctcctgctccacccccatcccactcccacccatgtctgggctcccaggcagggaacccgatctcttcctttgtgctggggccaggcgagtggagaaacgccctccagtctgagagcaggggagggaaggaggcagcagagttggggcagctgctcagagcagtgttctggcttcttctcaaaccctgagcgggctgccggcctccaagttcctccgacaagatgatggtactaattatggtacttttcactcactttgcacctttccctgtcgctctctaagcactttacctggatggcgcgtgggcagtgtgcaggcaggtcctgaggcctggggttggggtggagggtgcggcccggagttgtccatctgtccatcccaacagcaagacgaggatgtggctgttgagatgtgggccacactcacccttgtccaggatgcagggactgccttctccttcctgcttcatccggcttagcttggggctggctgcattcccccaggatgggcttcgagaaagacaaacttgtctggaaaccagagttgctgattccacccggggggcccggctgactcgcccatcacctcatctccctgtggacttgggagctctgtgccaggcccaccttgcggccctggctctgagtcgctctcccacccagcctggacttggccccatgggacccatcctcagtgctccctccagatcccgtccggcagcttggcgtccaccctgcacagcatcactgaatcacagagcctttgcgtgaaacagctctgccaggccgggagctgggtttctcttccctttttatctgctggtgtggaccacacctgggcctggccggaggaagagagagtttaccaagagagatgtctccgggcccttatttattatttaaacatttttttaaaaagcactgctagtttacttgtctctcctccccatcgtccccatcgtcctccttgtccctgacttggggcacttccaccctgacccagccagtccagctctgccttgccggctctccagagtagacatagtgtgtggggttggagctctggcacccggggaggtagcatttccctgcagatggtacagatgttcctgccttagagtcatctctagttccccacctcaatcccggcatccagccttcagtcccgcccacgtgctagctccgtgggcccaccgtgcggccttagaggtttccctccttcctttccactgaaaagcacatggccttgggtgacaaattcctctttgatgaatgtaccctgtggggatgtttcatactgacagattatttttatttattcaatgtcatatttaaaatatttattttttataccaaatgaatacttttttttttaagaaaaaaaagagaaatgaataaagaatctactcttggctggcaaaaaaaaaaaaaaaaaa

SEQ ID NO: 8 - protein, 213 aa, Homo sapiens
MNSFSTSAFGPVAFSLGLLLVLPAAFPAPVPPGEDSKDVAAPHRQPLTSSERIDKQIRYILDGISALRKETCNKSNMCESESKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 9 - protein, 136 aa, Homo sapiens
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 189 aa, Homo sapiens
MNSFSTIPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 12 - DNA, 2354 bp, Homo sapiens
  source 1..2354: mol_type = other DNA
gctcagggcacatgcctcccctccccaggccgcggcccagctgaccctcggggctcccccggcagcggacagggaagggttaaaggcccccggctccctgccccctgccctggggaacccctggccctgtggggacatgaactgtgtttgccgcctggtcctggtcgtgctgagcctgtggccagatacagctgtcgcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaat

SEQ ID NO: 13 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 14 - DNA, 1902 bp, Homo sapiens
  source 1..1902: mol_type = other DNA
agtctttgagagtcacatctcttatttggaccagtatagacagaagtaaacccagctgacttgtttcctgggacagttgagttaagggatggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtagcagttagtcccttctctcttccttgctttctcttctaatggaatatgcgtagttccctggggcctcgctttcccatcttaaatttctaaaaacagttaagacaacaggcattttctttcttttttctctgaccacctgcagcctgttgaaggactacaggtattttcatcaagtagcgttggagacatacacaaatgggcatacaagtttagcctggggggtgtgatttgtgtgcgtgcttgcatgtgctgcaggtgtaagagagtgggagcagggacaacgtccttccacttcagggttctaacctttctaacccactaagtaacctctacaggcatttaactgccttacagacagaatatacatatgttaattctagtcctggatgactcggtctgagaagattcaatttaaaatcagactctttagttgatttaaactcttagagaataagaataataatggctaacttttattatcttctatattaaggcagtatgccaagggtctttatgtatattatgtacagcgtttacaaccttgtgagcaaggtggtgttactcccattaggtagatgagaaaacaggctcacagagatttggttaagctcacacagctaacaagtagcacactgagtttgaacacagatcattctccttgtaaaagcctatgtgcctttcactttagaggcttgatcatgaatcactgcacctctttgtcacagggtgttggaagatgcatccatgtaatctattcccatcgctggaaaacagctgctgttagatgtcctcagaagtcagttgcaaattttagcgttaaagtcaggatttattgttcatacttggcggtgaggagggcagctggagatcttaagattccattttggaaaatgattaggcccgccaaacttctgaactttggaagctggggatgtttagtaatacagcctggtttttaagtactcactaaaagttctcaaatattgggttgggcacggcttataccaggttacctcacttttaattagtgatgcaggcagtgtaacccaagcatttgtggacaatgagtggaatactaaagttaaaaagtcaaactttcacctcagattttctggacttagtcatgaggagagggtgaggcccactctgttcctactggagataccagagactctgaaactatagaataaagcctctgtgctgcacaacaaa

SEQ ID NO: 15 - DNA, 2988 bp, Homo sapiens
  source 1..2988: mol_type = other DNA
gcaggagggcccagcgacgccgccgcgccagctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacggagttagacgaaaaaaaaaaaaaaaaaa

SEQ ID NO: 16 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 17 - DNA, 2217 bp, Homo sapiens
  source 1..2217: mol_type = other DNA
ccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggaaaaaaaaaaaaaaaaaa

SEQ ID NO: 18 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 19 - DNA, 5251 bp, Homo sapiens
  source 1..5251: mol_type = other DNA
cgcacacgcacacacacacacacacacacacacgcacgcacacacgtgtgcgcttctctgctccggagctgctgctgctcctgctctcagcgccgcagtggaaggcaggaccgaaccgctccttctttaaatatataaatttcagcccaggtcagcctcggcggcccccctcaccgcgctcccggcgcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcagaataagtctgtaagtttttttttttctttttaattgtaaatggttctttgtcagtttagtaaaccagtgaaatgttgaaatgttttgacatgtactggtcaaacttcagaccttaaaatattgctgtatagctatgctataggttttttcctttgttttggtatatgtaaccatacctatattattaaaatagatggatatagaagccagcataattgaaaacacatctgcagatctcttttgcaaactattaaatcaaaacattaactactttatgtgtaatgtgtaaatttttaccatattttttatattctgtaataatgtcaactatgatttagattgacttaaatttgggctctttttaatgatcactcacaaatgtatgtttcttttagctggccagtacttttgagtaaagcccctatagtttgacttgcactacaaatgcattttttttttaataacatttgccctacttgtgctttgtgtttctttcattattatgacataagctacctgggtccacttgtcttttcttttttttgtttcacagaaaagatgggttcgagttcagtggtcttcatcttccaagcatcattactaaccaagtcagacgttaacaaatttttatgttaggaaaaggaggaatgttatagatacatagaaaattgaagtaaaatgttttcattttagcaaggatttagggttctaactaaaactcagaatctttattgagttaagaaaagtttctctaccttggtttaatcaatatttttgtaaaatcctattgttattacaaagaggacacttcataggaaacatctttttctttagtcaggtttttaatattcagggggaaattgaaagatatatattttagtcgatttttcaaaaggggaaaaaagtccaggtcagcataagtcattttgtgtatttcactgaagttataaggtttttataaatgttctttgaaggggaaaaggcacaagccaatttttcctatgatcaaaaaattctttctttcctctgagtgagagttatctatatctgaggctaaagtttaccttgctttaataaataatttgccacatcattgcagaagaggtatcctcatgctggggttaatagaatatgtcagtttatcacttgtcgcttatttagctttaaaataaaaattaataggcaaagcaatggaatatttgcagtttcacctaaagagcagcataaggaggcgggaatccaaagtgaagttgtttgatatggtctacttcttttttggaatttcctgaccattaattaaagaattggatttgcaagtttgaaaactggaaaagcaagagatgggatgccataatagtaaacagcccttgtgttggatgtaacccaatcccagatttgagtgtgtgttgattatttttttgtcttccacttttctattatgtgtaaatcacttttatttctgcagacattttcctctcagataggatgacattttgttttgtattattttgtctttcctcatgaatgcactgataatattttaaatgctctattttaagatctcttgaatctgttttttttttttttaatttgggggttctgtaaggtctttatttcccataagtaaatattgccatgggaggggggtggaggtggcaaggaaggggtgaagtgctagtatgcaagtgggcagcaattatttttgtgttaatcagcagtacaatttgatcgttggcatggttaaaaaatggaatataagattagctgttttgtattttgatgaccaattacgctgtattttaacacgatgtatgtctgtttttgtggtgctctagtggtaaataaattatttcgatgatatgtggatgtctttttcctatcagtaccatcatcgagtctagaaaacacctgtgatgcaataagactatctcaagctggaaaagtcataccacctttccgattgccctctgtgctttctcccttaaggacagtcacttcagaagtcatgctttaaagcacaagagtcaggccatatccatcaaggatagaagaaatccctgtgccgtctttttattcccttatttattgctatttggtaattgtttgagatttagtttccatccagcttgactgccgaccagaaaaaatgcagagagatgtttgcaccatgctttggctttctggttctatgttctgccaacgccagggccaaaagaactggtctagacagtatcccctgtagccccataacttggatagttgctgagccagccagatataacaagagccacgtgctttctggggttggttgtttgggatcagctacttgcctgtcagtttcactggtaccactgcaccacaaacaaaaaaacccaccctatttcctccaatttttttggctgctacctacaagaccagactcctcaaacgagttgccaatctcttaataaataggattaataaaaaaagtaattgtgactcaaaaaaaaaaaaaa

SEQ ID NO: 20 - DNA, 5167 bp, Homo sapiens
  source 1..5167: mol_type = other DNA
cgcacacgcacacacacacacacacacacacacgcacgcacacacgtgtgcgcttctctgctccggagctgctgctgctcctgctctcagcgccgcagtggaaggcaggaccgaaccgctccttctttaaatatataaatttcagcccaggtcagcctcggcggcccccctcaccgcgctcccggcgcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcagaataagtctgtaagtttttttttttctttttaattgtaaatggttctttgtcagtttagtaaaccagtgaaatgttgaaatgttttgacatgtactggtcaaacttcagaccttaaaatattgctgtatagctatgctataggttttttcctttgttttggtatatgtaaccatacctatattattaaaatagatggatatagaagccagcataattgaaaacacatctgcagatctcttttgcaaactattaaatcaaaacattaactactttatgtgtaatgtgtaaatttttaccatattttttatattctgtaataatgtcaactatgatttagattgacttaaatttgggctctttttaatgatcactcacaaatgtatgtttcttttagctggccagtacttttgagtaaagcccctatagtttgacttgcactacaaatgcattttttttttaataacatttgccctacttgtgctttgtgtttctttcattattatgacataagctacctgggtccacttgtcttttcttttttttgtttcacagaaaagatgggttcgagttcagtggtcttcatcttccaagcatcattactaaccaagtcagacgttaacaaatttttatgttaggaaaaggaggaatgttatagatacatagaaaattgaagtaaaatgttttcattttagcaaggatttagggttctaactaaaactcagaatctttattgagttaagaaaagtttctctaccttggtttaatcaatatttttgtaaaatcctattgttattacaaagaggacacttcataggaaacatctttttctttagtcaggtttttaatattcagggggaaattgaaagatatatattttagtcgatttttcaaaaggggaaaaaagtccaggtcagcataagtcattttgtgtatttcactgaagttataaggtttttataaatgttctttgaaggggaaaaggcacaagccaatttttcctatgatcaaaaaattctttctttcctctgagtgagagttatctatatctgaggctaaagtttaccttgctttaataaataatttgccacatcattgcagaagaggtatcctcatgctggggttaatagaatatgtcagtttatcacttgtcgcttatttagctttaaaataaaaattaataggcaaagcaatggaatatttgcagtttcacctaaagagcagcataaggaggcgggaatccaaagtgaagttgtttgatatggtctacttcttttttggaatttcctgaccattaattaaagaattggatttgcaagtttgaaaactggaaaagcaagagatgggatgccataatagtaaacagcccttgtgttggatgtaacccaatcccagatttgagtgtgtgttgattatttttttgtcttccacttttctattatgtgtaaatcacttttatttctgcagacattttcctctcagataggatgacattttgttttgtattattttgtctttcctcatgaatgcactgataatattttaaatgctctattttaagatctcttgaatctgttttttttttttttaatttgggggttctgtaaggtctttatttcccataagtaaatattgccatgggaggggggtggaggtggcaaggaaggggtgaagtgctagtatgcaagtgggcagcaattatttttgtgttaatcagcagtacaatttgatcgttggcatggttaaaaaatggaatataagattagctgttttgtattttgatgaccaattacgctgtattttaacacgatgtatgtctgtttttgtggtgctctagtggtaaataaattatttcgatgatatgtggatgtctttttcctatcagtaccatcatcgagtctagaaaacacctgtgatgcaataagactatctcaagctggaaaagtcataccacctttccgattgccctctgtgctttctcccttaaggacagtcacttcagaagtcatgctttaaagcacaagagtcaggccatatccatcaaggatagaagaaatccctgtgccgtctttttattcccttatttattgctatttggtaattgtttgagatttagtttccatccagcttgactgccgaccagaaaaaatgcagagagatgtttgcaccatgctttggctttctggttctatgttctgccaacgccagggccaaaagaactggtctagacagtatcccctgtagccccataacttggatagttgctgagccagccagatataacaagagccacgtgctttctggggttggttgtttgggatcagctacttgcctgtcagtttcactggtaccactgcaccacaaacaaaaaaacccaccctatttcctccaatttttttggctgctacctacaagaccagactcctcaaacgagttgccaatctcttaataaataggattaataaaaaaagtaattgtgactcaaaaaaaaaaaaaa

SEQ ID NO: 21 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 22 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 23 - DNA, 3183 bp, Homo sapiens
  source 1..3183: mol_type = other DNA
gacagaagcaatggccgaggcagaagacaagccgaggtgctggtgaccctgggcgtctgagtggatgattggggctgctgcgctcagaggcctgcctccctgccttccaatgcatataaccccacaccccagccaatgaagacgagaggcagcgtgaacaaagtcatttagaaagcccccgaggaagtgtaaacaaaagagaaagcatgaatggagtgcctgagagacaagtgtgtcctgtactgcccccacctttagctgggccagcaactgcccggccctgcttctccccacctactcactggtgatctttttttttttacttttttttcccttttcttttccattctcttttcttattttctttcaaggcaaggcaaggattttgattttgggacccagccatggtccttctgcttcttctttaaaatacccactttctccccatcgccaagcggcgtttggcaatatcagatatccactctatttatttttacctaaggaaaaactccagctcccttcccactcccagctgccttgccacccctcccagccctctgcttgccctccacctggcctgctgggagtcagagcccagcaaaacctgtttagacacatggacaagaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcattttttttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggttcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

SEQ ID NO: 24 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

(19) United States
(12) Patent Application Publication
(10) Pub. No.: US 2024/0191206 A1
(43) Pub. Date: Jun. 13, 2024

(54) SERUM FREE MEDIA FOR SUSPENSION CULTURE OF MAMMALIAN LIVESTOCK PLURIPOTENT STEM CELLS

(71) Applicant: ACCELLTA LTD., Haifa (IL)

(72) Inventor: Michal AMIT, Yuvalim (IL)

(21) Appl. No.: 18/530,630

(22) Filed: Dec. 6, 2023

Related U.S. Application Data
(63) Continuation of application No. PCT/IL2022/050617, filed on Jun. 9, 2022.
(60) Provisional application No. 63/208,595, filed on Jun. 9, 2021.

(51) Int. Cl. C12N 5/074; A23J 3/22

(52) U.S. Cl. CPC C12N 5/0696; A23J 3/227; C12N 2500/25; C12N 2500/36; C12N 2500/90; C12N 2501/115; C12N 2501/13; C12N 2501/2306; C12N 2501/2311; C12N 2501/235; C12N 2501/415; C12N 2501/734; C12N 2506/45

(57) ABSTRACT

Provided are defined serum-free culture media comprising a basal medium, serum replacement and an effective concentration of at least one differentiation inhibiting agent, wherein the defined culture medium is capable of maintaining mammalian livestock pluripotent stem cells in an undifferentiated state for at least 5 passages in culture, wherein the basal medium is selected suitable for maintaining pluripotent stem cells in an undifferentiated state, wherein the serum replacement comprises insulin and transferrin, and wherein the serum replacement is devoid of selenium. Also provided are methods of maintaining mammalian livestock pluripotent stem cells in an undifferentiated state, comprising culturing the mammalian livestock pluripotent stem cells in the defined culture medium.

### RELATED APPLICATION/S

This application is a ByPass Continuation of PCT Patent Application No. PCT/IL2022/050617 having International filing date of Jun. 9, 2022, which claims the benefit of priority of U.S. Provisional Patent Application No. 63/208,595 filed on 9 Jun. 2021, the contents of which are all incorporated herein by reference in their entirety.

### SEQUENCE LISTING STATEMENT

The contents of the electronic sequence listing (ACLT-P-005-US ST26.xml; size: 53,228 bytes; and date of creation: Dec. 5, 2023) is herein incorporated by reference in its entirety.

### FIELD AND BACKGROUND OF THE INVENTION

The present invention, in some embodiments thereof, relates to defined culture media suitable for expansion of mammalian pluripotent stem cells (such as mammalian livestock pluripotent stem cells) in an undifferentiated state and, more particularly, but not exclusively, to cell cultures comprising same and methods of expanding mammalian pluripotent stem cells using same.

In recent years, extensive investigation into improving the culture systems for pluripotent stem cells (PSCs) has yielded three main advances: (1) the ability to grow cells in serum-free conditions [Amit et al, 2000]; (2) prolonged culture of PSCs in feeder-layer-free conditions without the addition of mouse embryonic fibroblasts (MEF)-conditioned medium, while using selected growth factors [Amit et al, 2004; Xu et al, 2005; Xu et al, 2005b and Ludwig et al 2006], and (3) culturing PSCs in suspension cultures (Amit et al, 2010, 2011).

Several recent studies discussed the possible involvement of several intracellular transduction pathways in PSC renewal and maintenance of “stemness” identity, but the mechanism underlining embryonic stem cell (ESC) self-maintenance is still unrevealed. In some culture methods PSCs can be culture continuously without feeder layers provided that the culture medium is supplemented with factors cocktail including Wnt3a, basic fibroblast growth factor (bFGF) and transforming growth factor (TGF) beta-1 [Xu et al 2005, Hanna et al 2007; Amit et al, 2004; Ludwig et al 2006: Ross 2019]. Suspension culture methods are based on medium supplemented with Wnt3a and IL6RIL6 chimera or leukemia inhibitory factor (LIF), without bFGF or combinations of bFGF and gp130 agonists [Amit et al 2010 and Amit et al 2011]. Traditionally, culturing of PSC in two-dimensional or three-dimensional culture systems involves the addition of a culture medium which includes 15-20% of serum or serum replacement such as knockout Serum Replacement (Life technology). Currently known serum replacement formulations include insulin, transferrin, selenium, albumin and fatty acids [Amit et al 2000; Life technology ko-SR instructions].

Additional background art includes Seyed Mohamad Javad Taher-Mofrad et al., 2020 (Cryobiology, 92:208-214); Sadegh Ghorbani-Dalini et al., 2020 (3 Biotech, 10: 215).

### SUMMARY OF THE INVENTION

According to an aspect of some embodiments of the present invention there is provided a defined serum-free culture medium comprising a basal medium, serum replacement and an effective concentration of at least one differentiation inhibiting agent, wherein the defined culture medium is capable of maintaining mammalian livestock pluripotent stem cells in an undifferentiated state for at least 5 passages in culture, wherein the basal medium is selected suitable for maintaining pluripotent stem cells in an undifferentiated state, wherein the serum replacement comprises insulin and transferrin, and wherein the serum replacement is devoid of selenium.

According to some embodiments of the invention, the insulin is provided at a concentration in a range of 0.34×10⁻³ mM to 1.88×10⁻³ mM, and wherein the transferrin is provided at a concentration in a range of 0.137×10⁻⁴ mM to 0.66×10⁻⁴ mM.

According to an aspect of some embodiments of the present invention there is provided a defined serum-free culture medium comprising a basal medium, serum replacement and an effective concentration of at least one differentiation inhibiting agent, wherein the defined culture medium is capable of maintaining mammalian livestock pluripotent stem cells in an undifferentiated state for at least 5 passages in culture, wherein the basal medium is selected suitable for maintaining pluripotent stem cells in an undifferentiated state, wherein the serum replacement comprises insulin and transferrin, wherein the insulin is provided at a concentration in a range of 0.34×10⁻³ mM to 1.88×10⁻³ mM, and wherein the transferrin is provided at a concentration in a range of 0.137×10⁻⁴ mM to 0.66×10⁻⁴ mM.

According to an aspect of some embodiments of the present invention there is provided a cell culture comprising cells and the defined culture medium of some embodiments of the invention.

According to an aspect of some embodiments of the present invention there is provided a method of maintaining mammalian livestock pluripotent stem cells in an undifferentiated state, comprising culturing the mammalian livestock pluripotent stem cells in the defined culture medium of some embodiments of the invention.

According to an aspect of some embodiments of the present invention there is provided a method of differentiating mammalian livestock pluripotent stem cells comprising:
(a) culturing the mammalian livestock pluripotent stem cells according to the method of some embodiments of the invention, to thereby obtain an expanded population of mammalian livestock pluripotent stem cells in an undifferentiated state, and
(b) culturing the expanded population of mammalian livestock pluripotent stem cells in an undifferentiated state under conditions devoid of the differentiation inhibiting agent which allow differentiation of the mammalian livestock pluripotent stem cells,
thereby differentiating the mammalian livestock pluripotent stem cells.

According to an aspect of some embodiments of the present invention there is provided a method of preparing a food product, comprising combining differentiated mammalian livestock cells resultant from the method of some embodiments of the invention with a food product, thereby preparing the food product.

According to an aspect of some embodiments of the present invention there is provided a food product comprising differentiated mammalian livestock cells resultant from the method of some embodiments of the invention.

According to some embodiments of the invention, the defined culture medium of some embodiments of the invention, with the proviso that the basal medium is not RPMI1640.

According to some embodiments of the invention, the basal medium is selected from the group consisting of KO-DMEM, DMEM/F12 and DMEM.

According to some embodiments of the invention, the basal medium is selected from the group consisting of KO-DMEM and DMEM/F12.

According to some embodiments of the invention, the basal medium is provided at a concentration in a range of 93-98%.

According to some embodiments of the invention, the basal medium is provided at a concentration in a range of 94-96%.

According to some embodiments of the invention, the culture medium is devoid of a cryoprotectant.

According to some embodiments of the invention, the culture medium further comprises selenium.

According to some embodiments of the invention, the culture medium does not comprise selenium.

According to some embodiments of the invention, the culture medium further comprises a lipid mixture at a concentration range of 0.5-1.2% (v/v).

According to some embodiments of the invention, the serum replacement further comprises a lipid selected from the group consisting of. Linoleic Acid at a concentration in a range of 0.47-0.63×10⁻⁴ mM, Lipoic Acid at a concentration in a range of 1-1.33×10⁻⁴ mM, Arachidonic Acid at a concentration in a range of 0.32-0.43×10⁻⁵ mM, Cholesterol at a concentration in a range of 0.28-0.37×10⁻³ mM, DL-alpha tocopherol-acetate at a concentration in a range of 0.72-0.96×10⁻³ mM, Linolenic Acid at a concentration in a range of 1.74-2.33×10⁻⁵ mM Myristic Acid at a concentration in a range of 2.14-2.86×10⁻⁵ mM, Oleic Acid at a concentration in a range of 1.73-2.31×10⁻⁵ mM, Palmitic Acid at a concentration in a range of 1.91-2.55×10⁻⁵ mM, Palmitoleic acid at a concentration in a range of 1.92-2.571×10⁻⁵ mM, and Stearic Acid at a concentration in a range of 1.72-2.29×10⁻⁵ mM.

According to some embodiments of the invention, the serum replacement further comprises ascorbic acid at a concentration in a range of 125-170 mM.

According to some embodiments of the invention the serum replacement further comprises ascorbic acid at a concentration in a range of 8-17 micrograms/milliliter.

According to some embodiments of the invention, the serum replacement further comprises bovine serum albumin at a concentration in a range of 0.4% to 0.7% volume/volume (v/v).

According to some embodiments of the invention, the bovine serum albumin is at a concentration in a range of 0.5% to 0.66% volume/volume (v/v).

According to some embodiments of the invention, the serum replacement is knockout (KO)-serum replacement provided at a concentration in a range of 1-10% volume/volume (v/v).

According to some embodiments of the invention, the at least one differentiation inhibiting agent is a growth factor, a cytokine, a small molecule, or a combination thereof, wherein the effective concentration of the at least one differentiation inhibiting agent is capable of maintaining the mammalian livestock pluripotent stem cells in an undifferentiated states for at least 5 passages in culture.

According to some embodiments of the invention, the growth factor is basic fibroblast growth factor (bFGF).

According to some embodiments of the invention, the effective concentration of the bFGF in the defined culture medium of some embodiments of the invention is in a range of 4-110 ng/ml.

According to some embodiments of the invention, the effective concentration of the bFGF in the defined culture medium of some embodiments of the invention is about 50 ng/ml.

According to some embodiments of the invention, the effective concentration of the bFGF in the defined culture medium of some embodiments of the invention is about 10 ng/ml.

According to some embodiments of the invention, the effective concentration of the bFGF in the defined culture medium of some embodiments of the invention is about 100 ng/ml bFGF.

According to some embodiments of the invention, the at least one differentiation inhibiting agent is the IL6RIL6 chimera.

According to some embodiments of the invention, the effective concentration of the IL6RIL6 chimera in the defined culture medium of some embodiments of the invention is about 100 pg/ml.

According to some embodiments of the invention, the at least one differentiation inhibiting agent is a gp130 agonist.

According to some embodiments of the invention, the gp130 agonist is selected from the group consisting of leukemia inhibitory factor (LIF), interleukin-6 (IL6), interleukin-11 (IL11), and Ciliary neurotrophic factor (CNTF).

According to some embodiments of the invention, the effective concentration of the LIF in the defined culture medium of some embodiments of the invention is about 3000 U/ml (units per milliliter).

According to some embodiments of the invention, the effective concentration of the IL6 in the defined culture medium of some embodiments of the invention is about 100 ng/ml.

According to some embodiments of the invention, the effective concentration of the IL11 in the defined culture medium of some embodiments of the invention is about 1 ng/ml.

According to some embodiments of the invention, the effective concentration of the CNTF in the defined culture medium of some embodiments of the invention is about 1 ng/ml.

According to some embodiments of the invention, the defined culture medium of some embodiments of the invention, further comprises ascorbic acid.

According to some embodiments of the invention, the ascorbic acid is at a concentration range of 8-600 μg/ml.

According to some embodiments of the invention, the ascorbic acid is at a concentration range of 10-600 μg/ml.

According to some embodiments of the invention, the ascorbic acid is at a concentration range of 450-550 μg/ml.

According to some embodiments of the invention, the defined culture medium of some embodiments of the invention, wherein the culture medium comprises ascorbic acid at a concentration range of 450-550 μg/ml and basic fibroblast growth factor at a concentration of 40-60 ng/ml.

According to some embodiments of the invention, the at least one differentiation inhibiting agent comprises leukemia inhibitory factor (LIF) at a concentration of about 3000 U/ml and basic fibroblast growth factor (bFGF) at a concentration of about 50 ng/ml.

According to some embodiments of the invention, the at least one differentiation inhibiting agent comprises leukemia inhibitory factor (LIF) at a concentration of about 3000 U/ml and basic fibroblast growth factor (bFGF) at a concentration of about 10 ng/ml.

According to some embodiments of the invention, the at least one differentiation inhibiting agent comprises a Wnt3a polypeptide and basic fibroblast growth factor (bFGF).

According to some embodiments of the invention, the effective concentration of the Wnt3a polypeptide in the defined culture medium of some embodiments of the invention is about 10 ng/ml.

According to some embodiments of the invention, the effective concentration of the bFGF in the defined culture medium of some embodiments of the invention is in a range of 4-100 ng/ml.

According to some embodiments of the invention, the effective concentration of the bFGF in the defined culture medium of some embodiments of the invention is about 100 ng/ml.

According to some embodiments of the invention, the small molecule is a protease inhibitor selected from the group consisting of: phenylmethylsulfonyl fluoride (PMSF) and Tosyl-L-lysyl-chloromethane hydrochloride (TLCK).

According to some embodiments of the invention, the at least one differentiation inhibiting agent further comprises the IL6RIL6 chimera.

According to some embodiments of the invention, the effective concentration of the IL6RIL6 chimera in the defined culture medium of some embodiments of the invention is in a range of 80-120 μg/ml.

According to some embodiments of the invention, the effective concentration of the PMSF in the defined culture medium of some embodiments of the invention in a range of 70-130 μM.

According to some embodiments of the invention, the effective concentration of the TLCK in the defined culture medium of some embodiments of the invention is in a range of 20-80 μM.

According to some embodiments of the invention, the at least one differentiation inhibiting agent comprises a gp130 agonist selected from the group consisting of leukemia inhibitory factor (LIF), interleukin-6 (IL6), interleukin-11 (IL11), and Ciliary neurotrophic factor (CNTF) and a protease inhibitor selected from the group consisting of phenylmethylsulfonyl fluoride (PMSF) and Tosyl-L-lysyl-chloromethane hydrochloride (TLCK).

According to some embodiments of the invention, the at least one differentiation inhibiting agent comprises a Wnt3a polypeptide and the IL6RIL6 chimera.

According to some embodiments of the invention, the effective concentration of the Wnt3a polypeptide in the medium is in a range of 5-20 ng/ml, and wherein the effective concentration of the IL6RIL6 chimera in the medium is in a range of 70-130 μg/ml.

According to some embodiments of the invention, the at least one differentiation inhibiting agent comprises basic fibroblast growth factor (bFGF) and transforming growth factor beta 1 (TGFβ1).

According to some embodiments of the invention, the effective concentration of the TGFβ1 in the defined culture medium of some embodiments of the invention is about 0.12 ng/ml.

According to some embodiments of the invention, the effective concentration of the bFGF in the defined culture medium of some embodiments of the invention is about 10 ng/ml.

According to some embodiments of the invention, the effective concentration of the bFGF in the defined culture medium of some embodiments of the invention is about 100 ng/ml.

According to some embodiments of the invention, the cells are mammalian livestock pluripotent stem cells.

According to some embodiments of the invention, the method further comprising passaging the mammalian livestock pluripotent stem cells for at least one time.

According to some embodiments of the invention, the passaging is effected every 5-21 days during the culturing.

According to some embodiments of the invention, the passaging comprises splitting the mammalian livestock pluripotent stem cells in a 1 to 2, or a 2 to 3 ratio before further culturing the cells.

According to some embodiments of the invention, the culturing is performed on feeder cell layers.

According to some embodiments of the invention, the culturing is performed on a feeder-free matrix.

According to some embodiments of the invention, the culturing is performed in a suspension culture devoid of substrate adherence.

According to some embodiments of the invention, the conditions comprise culturing the cells in a culture medium suitable for differentiating the mammalian livestock undifferentiated stem cells into muscle cells.

According to some embodiments of the invention, the conditions comprise culturing the cells in a culture medium suitable for differentiating the mammalian livestock undifferentiated stem cells into blood cells.

According to some embodiments of the invention, the conditions comprise culturing the cells in a culture medium suitable for differentiating the mammalian livestock undifferentiated stem cells into fat cells.

According to some embodiments of the invention, the conditions comprise culturing the cells in a culture medium suitable for differentiating the mammalian livestock undifferentiated stem cells into connective tissue cells.

According to some embodiments of the invention, the culturing in steps (a) and (b) is performed in a suspension culture.

According to some embodiments of the invention, the culturing in the suspension culture is without adherence to a substrate.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E are photographs showing the morphology of undifferentiated Bovine PSC cells. FIGS. 1A-B depict undifferentiated iPSCs colonies of cell line iBVN 1.14 p7+23 cultured on MEFs for 5 passages with the YF10 medium supplemented with 5% KoSR (KNOCKOUT™ serum replacement (Gibco-Invitrogen Corporation)). FIGS. 1C-E depict undifferentiated PSCs colonies cultured on MEFs for the indicated number of passages in the indicated culture media: FIG. 1C—BVN4 P8 cultured for 7 passages with the IL6RIL6 Chimera medium (with 50 ng/ml bFGF) supplemented with 5% KoSR; FIG. 1D—BVN3 P5, cultured for 5 passages (since derivation) in the IL6RIL6 Chimera medium (with 50 ng/ml bFGF) supplemented with 5% KoSR; FIG. 1E—iBVN1.14 P7+29 cultured for 6 passages with the Wnt3a+IL6RIL6 Chimera (with 50 ng/ml bFGF) supplemented with 5% KoSR. Scale bars: FIG. 1A=200 μm; FIG. 1B=100 μm. FIGS. 1C, D, and E=100 μm;

FIGS. 2A-D are images depicting immunofluorescence analyses for expression of TRA-1-60 and TRA-1-81 in undifferentiated iPSCs colonies. iBVN 1.4 p7+27 cells were cultured on MEFs for 8 passages with the YF10 medium supplemented with 10% KoSR medium. Prior to immunofluorescence (IF) analysis the cells were fixed with methanol. As shown by the IF analysis, most of cells were positively stained to TRA 1-60 (in Red, FIG. 2B) and TRA-1-81 (in Green, FIG. 2D). Nuclei were counterstained with DAPI (Blue, FIGS. 2A-B). Scale bars: 100 μm;

FIGS. 3A-D are images depicting expression of Nanog and TRA-1-60 in undifferentiated iPSCs colonies. iBVN 1.4 p7+30 cells were cultured on MEFs for 11 passages with the YF10 medium supplemented with 5% KoSR medium. Prior to immunofluorescence (IF) analysis the cells were fixed with 4% paraformaldehyde (PFA) for Nanog staining, or with methanol for TRA-1-60 staining. As shown by the IF analysis, most of the iBVN 1.4 p7+30 cells were positively stained to Nanog (Green, FIG. 3B) and TRA-1-60 (Red, FIG. 3D). Nuclei were counterstained with DAPI (Blue, FIG. 3A and FIG. 3C). Scale bars: FIGS. 3A-B=100 μm; FIGS. 3C-D=50 μm;

FIGS. 4A-B are images depicting expression of TRA-1-81 in undifferentiated iPSCs colonies. iBVN 1.4 p7+30 were cultured on MEFs for 11 passages with the YF10 medium supplemented with 5% KoSR medium. Prior to immunofluorescence (IF) analysis the cells were fixed with methanol. As shown by the IF analysis, most of the cells were positively stained to TRA-1-81 (Green, FIG. 4B). Nuclei were counterstained with DAPI (Blue, FIG. 4A). Scale bars: FIGS. 4A-B=100 μm;

FIGS. 5A-D are images depicting expression of TRA-1-60 and TRA-1-81 in undifferentiated iPSCs colonies. iBVN 1.4 p7+30 cells were cultured on MEFs for 11 passages with the IL6RIL6 Chimera medium (with 50 ng/ml bFGF) supplemented with 5% KoSR. Prior to immunofluorescence (IF) analysis the cells were fixed with methanol. As shown by the IF analysis, most of the cells were positively stained to TRA-1-60 (Red, FIG. 5B) and TRA-1-81 (Green, FIG. 5D). Nuclei were counterstained with DAPI (Blue, FIGS. 5A and 5C). Scale bars: FIGS. 5A-B=100 μm; FIGS. 5C-D=50 μm;

FIGS. 6A-D are images depicting expression of TRA-1-60 and TRA-1-81 in undifferentiated iPSCs colonies. iBVN 1.14 p7+29 cells were cultured on MEFs for 10 passages with the IL6RIL6 Chimera medium (with 50 ng/ml bFGF) supplemented with 5% KoSR. Prior to immunofluorescence (IF) analysis the cells were fixed with methanol. As shown by the IF analysis, most of the cells were positively stained to TRA-1-60 (Red, FIG. 6B) and TRA-1-81 (Green, FIG. 6D). Nuclei were counterstained with DAPI (Blue, FIGS. 6A and 6C). Scale bars: FIGS. 6A-D=100 μm;

FIG. 7 is an image depicting the morphology of undifferentiated Bovine iPSC cells cultured in suspension. Undifferentiated iPSCs line iBVN1.4 p7+27 were cultured in suspension for one month with the IL6RIL6 Chimera medium (with 50 ng/ml bFGF) supplemented with 10% KoSR. Scale bar=50 μm;

FIG. 8 is an image depicting the morphology of undifferentiated Bovine iPSC cells cultured in suspension and re-plated on MEFs. Undifferentiated iPSCs line iBVN1.4 p7+17 were cultured in suspension for one month with the IL6RIL6 Chimera medium (with 50 ng/ml bFGF) supplemented with 5% KoSR, and were then re-plated on MEFs. All aggregates were attached and form colonies with undifferentiated cells morphology. Scale bar=100 μm;

FIGS. 9A-D are images depicting expression of Nanog and TRA-1-60 in undifferentiated iPSCs colonies. iBVN 1.14 p7+30 cells were cultured in suspension for 1 month with the IL6RIL6 Chimera medium (with 50 ng/ml bFGF) supplemented with 5% KoSR. Cells were then re-plated on MEF inactivated before IF staining. Prior to immunofluorescence (IF) analysis the cells were fixed with methanol. As shown by the IF analysis, most of the cells were positively stained to Nanog (Green, FIG. 9B) and TRA-1-60 (Red, FIG. 9D). Nuclei were counterstained with DAPI (Blue, FIGS. 9A and 9C). Scale bars: FIGS. 9A-D=100 μm;

FIGS. 10A-B are images depicting expression of TRA-1-81 in undifferentiated iPSCs colonies. iBVN 1.14 p7+30 cells were cultured in suspension for passages 1 month with the IL6RIL6 Chimera medium (with 50 ng/ml bFGF) and 5% KoSR. Cells were then re-plated on MEF inactivated before IF staining. Prior to immunofluorescence (IF) analysis the cells were fixed with methanol. As shown by the IF analysis, most of the cells were positive for TRA-1-81 (Green, FIG. 10B). Nuclei were counterstained with DAPI (Blue, FIG. 10A). Scale Bar: FIGS. 10A-B=100 μm.

FIGS. 11A-D are images depicting the morphology of undifferentiated Bovine iPSC cells in the presence of 15% KoSR. iBVN1.4 p7+42 cells were cultured on MEFs for 3 passages with the IL6RIL6 Chimera medium (with 50 ng/ml bFGF) supplemented with 15% KoSR. Some of the colonies differentiated, mainly to fat cells (FIGS. 11A and 11C). In other colonies areas with fat cells (marked with white arrow) could be noted (FIGS. 11B and 11D). Differentiation into adipocytes was confirmed with the oil red staining (FIGS. 11C and 11D). Scale bars: FIGS. 11A-B=100 μm; FIGS. 11C-D=50 μm;

FIGS. 12A-D are images depicting the morphology of undifferentiated Bovine iPSC cells in the presence of 10% KoSR. iBVN1.4 p7+42 cells were cultured on MEFs for 3 passages in the presence of the IL6RIL6 Chimera medium (with 50 ng/ml bFGF) supplemented with 10% KoSR. Some of the colonies differentiated, mainly to fat cells (FIGS. 12A and 12C). In other colonies areas with fat cells (marked with white arrow) could be noted (FIGS. 12B and 12D). Differentiation into adipocytes was confirmed with oil red staining (FIGS. 12C and 12D). Scale bars: FIGS. 12A-B=100 μm; FIGS. 12C-D=50 μm.

FIGS. 13A-B—are images depicting the morphology of undifferentiated Bovine iPSC cells cultured on MEFs in the presence of 1-2.5% KoSR. iBVN1.4 p7+43 cells were cultured on MEFs with the IL6RIL6 Chimera medium (with 50 ng/ml bFGF) supplemented with 2.5% KoSR for 7 passages (FIG. 13A) or with 1% KoSR for 7 passages (FIG. 13B). The colonies remained in the undifferentiated state for at least 13 passages with less than 3% differentiated cells. Scale bar: FIGS. 13A-B=100 μm;

FIGS. 14A-C are images depicting derivation of BVN3 cell line in the IL6RIL6 chimera medium (with 50 ng/ml bFGF) supplemented with 5% KoSR. ESC line BVN3 was derived using a whole embryo approach. FIG. 14A—Embryo before Zona pellucida removal. FIG. 14B—Embryo (Day 8) after Zona pellucida removal with Tyrode acid. ICM is clearly noted (white arrow). FIG. 14C—ICM outgrowth. Scale bars: FIGS. 14A-B=50 μm; FIG. 14C=200 μm;

FIG. 15 is a histogram depicting the diameter of the colonies of bovine pluripotent stem cells that were cultured on MEFs with the IL6RIL6 chimera (with 50 ng/ml bFGF) medium and with different concentrations of KoSR. iBVN1.4 line at passage 42 and 43 was used in this experiment. The cells were cultured for the indicated number of passages in the IL6RIL6 chimera (with 50 ng/ml bFGF) medium which included the following concentrations of KoSR: 15% KoSR—for 3 passages; 10% KoSR—for 3 passages; 7.5% KoSR—for 3 passages; 5% KoSR—for 3 passages; 2.5% KoSR—for 7 passages and 1% KoSR—for 7 passages. The diameters of colonies were measured three days post splitting of the cells. It is noted that at concentrations of 1% or 2.5% of KoSR the average diameter of colonies is smaller as compared to the diameter of colonies grown in the same medium supplemented with 5% KoSR or with higher concentrations of KoSR such as 7.5%, 10% or 15%. No significant difference was found between concentrations of 5-15% KoSR.

FIGS. 16A-B are images depicting expression of TRA1-60 and Nanog in undifferentiated bovine PSCs (iBVN 1.4) which were cultured in a CNTF and IL-11 medium on a two-dimensional culture system. Bovine PSCs were cultured in 2D (on mouse embryonic fibroblasts (MEF) feeder cells) with a culture media supplemented with CNTF (1 ng/ml) and IL11 (1 ng/ml) and following 3 passages in culture the cells were subjected to immunofluorescence analysis for the key pluripotency markers TRA1-60 (FIG. 16A) and Nanog (FIG. 16B). FIG. 16A: TRA1-60 (orange color), DAPI (nuclei staining, blue color) and a merged image (“Merge”, with a double staining of orange and blue colors showing pluripotent stem cells). FIG. 16B: Nanog (green color), DAPI (nuclei staining, blue color) and a merged image (“Merge”, with a double staining of green and blue colors showing pluripotent stem cells). The results show positive staining for TRA1-60 and Nanog, demonstrating that a medium supplemented with CNTF and IL-11 supports pluripotency of bovine PSCs for at least 3 passages while cultured on a 2D culture system. Magnification ×10 (TRA1-60), ×20 (Nanog).

FIGS. 17A-C are images depicting expression of OCT4, Nanog and SSEA1 in undifferentiated porcine PSCs (Psus 1) which were cultured in a CNTF and IL-11 medium in a three-dimensional suspension culture. Porcine PSCs were cultured in 3D with a culture media supplemented with CNTF (1 ng/ml) and IL11 (1 ng/ml) and following 3 passages in culture the cells were subjected to immunofluorescence analysis for the key pluripotency markers OCT4 (FIG. 17A), Nanog (FIG. 17B) and SSEA1 (FIG. 17C). FIG. 17A: OCT4 (red color), DAPI (nuclei staining, blue color) and a merged image (“Merge”, with a double staining of red and blue colors showing pluripotent stem cells). FIG. 17B: Nanog (green color), DAPI (nuclei staining, blue color) and a merged image (“Merge”, with a double staining of green and blue colors showing pluripotent stem cells). FIG. 17C: SSEA1 (orange color), DAPI (nuclei staining, blue color) and a merged image (“Merge”, with a double staining of orange and blue colors showing pluripotent stem cells). The results show positive staining for OCT4, Nanog, and SSEA1, demonstrating that a medium supplemented with CNTF and IL-11 supports pluripotency of porcine PSCs for at least 3 passages while cultured on a 3D suspension culture. Magnification ×20.

FIGS. 18A-B are images depicting expression of TRA1-60 and Nanog in undifferentiated bovine PSCs (iBVN 1.4) which were cultured in a PMSF medium with a concentration of 70 μM PMSF on a two-dimensional culture system. Bovine PSCs were cultured in 2D (on MEFs feeder cells) with a culture media supplemented with PMSF at a concentration of 70 μM and following 3 passages in culture the cells were subjected to immunofluorescence analysis for the key pluripotency markers TRA1-60 (FIG. 18A) and Nanog (FIG. 18B). FIG. 18A: TRA1-60 (orange color), DAPI (nuclei staining, blue color) and a merged image (“Merge”, with a double staining of orange and blue colors showing pluripotent stem cells). FIG. 18B: Nanog (green color), DAPI (nuclei staining, blue color) and a merged image (“Merge”, with a double staining of green and blue colors showing pluripotent stem cells). The results show positive staining for TRA1-60 and Nanog, demonstrating that a medium supplemented with PMSF 70 μM supports pluripotency of bovine PSCs for at least 3 passages while cultured on a 2D culture system. Magnification ×10 (TRA1-60), ×20 (Nanog).

FIG. 19 shows images depicting expression of TRA1-60 in undifferentiated bovine PSCs (iBVN 1.4) which were cultured in a PMSF medium with a concentration of 130 μM PMSF on a two-dimensional culture system. Bovine PSCs were cultured in 2D (on MEFs feeder cells) with a culture media supplemented with PMSF at a concentration of 130 μM and following 3 passages in culture the cells were subjected to immunofluorescence analysis for the key pluripotency marker TRA1-60. Shown are images of TRA1-60 (orange color) staining, DAPI (nuclei staining, blue color) and a merged image (“Merge”, with a double staining of orange and blue colors showing pluripotent stem cells). The results show positive staining for TRA1-60, demonstrating that a medium supplemented with PMSF 130 μM supports pluripotency of bovine PSCs for at least 3 passages while cultured on a 2D culture system. Magnification ×20.

FIGS. 20A-C are images depicting expression of OCT4, Nanog and SSEA1 in undifferentiated porcine PSCs (Psus 1) which were cultured in a PMSF medium with a concentration of 100 μM PMSF in a three-dimensional suspension culture. Porcine PSCs were cultured in 3D with a culture media supplemented with PMSF at a concentration of 100 μM and following 3 passages in culture the cells were subjected to immunofluorescence analysis for the key pluripotency markers OCT4 (FIG. 20A), Nanog (FIG. 20B) and SSEA1 (FIG. 20C). FIG. 20A: OCT4 (red color), DAPI (nuclei staining, blue color) and a merged image (“Merge”, with a double staining of red and blue colors showing pluripotent stem cells). FIG. 20B: Nanog (green color), DAPI (nuclei staining, blue color) and a merged image (“Merge”, with a double staining of green and blue colors showing pluripotent stem cells). FIG. 20C: SSEA1 (orange color), DAPI (nuclei staining, blue color) and a merged image (“Merge”, with a double staining of orange and blue colors showing pluripotent stem cells). The results show positive staining for OCT4, Nanog and SSEA1, demonstrating that the medium supplemented with PMSF at a concentration of 100 μM supports pluripotency of porcine PSCs for at least 3 passages while cultured on a 3D suspension culture. Magnification ×20.

FIGS. 21A-B are images depicting expression of OCT4 and SSEA1 in undifferentiated porcine PSCs (Psus 1) which were cultured in a PMSF medium with a concentration of 100 μM PMSF on a two-dimensional culture system. Porcine PSCs were cultured in 2D (on MEFs feeder cells) with a culture media supplemented with PMSF at a concentration of 100 μM and following 3 passages the cells were subjected to immunofluorescence analysis for the key pluripotency markers OCT4 (FIG. 21A) and SSEA1 (FIG. 21B). FIG. 21A: OCT4 (red color), DAPI (nuclei staining, blue color) and a merged image (“Merge”, with a double staining of red and blue colors showing pluripotent stem cells). FIG. 21B: SSEA1 (orange color), DAPI (nuclei staining, blue color) and a merged image (“Merge”, with a double staining of orange and blue colors showing pluripotent stem cells). The results show positive staining for OCT4 and SSEA1, demonstrating that a medium supplemented with PMSF 100 μM supports pluripotency of porcine PSCs for at least 3 passages while cultured on a 2D culture system. Magnification ×20.

FIG. 22 shows images depicting expression of SSEA1 in undifferentiated porcine PSCs (Psus 1) which were cultured in a CNTF and IL11 medium on a two-dimensional culture system. Porcine PSCs were cultured in 2D (on MEFs feeder cells) with a culture media supplemented with CNTF (1 ng/ml) and IL11 (1 ng/ml) and following 3 passages in culture the cells were subjected to immunofluorescence analysis for the key pluripotency marker SSEA1. Shown are images of SSEA1 staining (orange color), DAPI (nuclei staining, blue color) and a merged image (“Merge”, with a double staining of orange and blue colors showing pluripotent stem cells). The results show positive staining for SSEA1, demonstrating that a medium supplemented with CNTF and IL11 supports pluripotency of porcine PSCs for at least 3 passages while cultured on a 2D culture system. Magnification ×20.

FIGS. 23A-D are images depicting expression of OCT4, Nanog, TRA1-60 and TRA1-81 in undifferentiated bovine PSCs (iBVN 1.4) which were cultured in a CNTF and IL11 medium on a two-dimensional culture system. Bovine PSCs were cultured in 2D (on MEFs feeder cells) with a culture media supplemented with CNTF (1 ng/ml) and IL11 (1 ng/ml) and following 5 passages in culture the cells were subjected to immunofluorescence analysis for the key pluripotency markers OCT4 (FIG. 23A), Nanog (FIG. 23B), TRA1-60 (FIG. 23C) and TRA1-81 (FIG. 23D). FIG. 23A: OCT4 (red color), DAPI (nuclei staining, blue color) and a merged image (“Merge”, with a double staining of red and blue colors showing pluripotent stem cells). FIG. 23B: Nanog (green color), DAPI (nuclei staining, blue color) and a merged image (“Merge”, with a double staining of green and blue colors showing pluripotent stem cells). FIG. 23C: TRA1-60 (orange color), DAPI (nuclei staining, blue color) and a merged image (“Merge”, with a double staining of orange and blue colors showing pluripotent stem cells). FIG. 23D: TRA1-81 (green color), DAPI (nuclei staining, blue color) and a merged image (“Merge”, with a double staining of green and blue colors showing pluripotent stem cells). The results show positive staining for OCT4, Nanog, TRA1-60 and TRA1-81, demonstrating that a medium supplemented with CNTF and IL11 supports pluripotency of bovine PSCs for at least 5 passages while cultured on a 2D culture system. Magnification ×20.

FIGS. 24A-B are images depicting expression of OCT4 and SSEA1 in undifferentiated porcine PSCs (Psus 1) which were cultured in a CNTF and IL11 medium in a three-dimensional suspension culture. Porcine PSCs were cultured in 3D with a culture media supplemented with CNTF (1 ng/ml) and IL11 (1 ng/ml) and following 5 passages in culture the cells were subjected to immunofluorescence analysis for the key pluripotency markers OCT4 (FIG. 24A) and SSEA1 (FIG. 24B). FIG. 24A: OCT4 (red color), DAPI (nuclei staining, blue color) and a merged image (“Merge”, with a double staining of red and blue colors showing pluripotent stem cells). FIG. 24B: SSEA1 (orange color), DAPI (nuclei staining, blue color) and a merged image (“Merge”, with a double staining of orange and blue colors showing pluripotent stem cells). The results show positive staining for OCT4 and SSEA1, demonstrating that the medium supplemented with CNTF and IL11 supports pluripotency of porcine PSCs for at least 5 passages while cultured on a 3D suspension culture. Magnification OCT4 (×20), SSEA1 (×10).

FIGS. 25A-B are images depicting expression of OCT4 and SSEA1 in undifferentiated porcine PSCs (Psus 1) which were cultured in a PMSF medium with a concentration of 100 μM PMSF in a three-dimensional suspension culture. Porcine PSCs were cultured in 3D with a culture media supplemented with PMSF at a concentration of 100 μM and following 5 passages in culture the cells were subjected to immunofluorescence analysis for the key pluripotency markers OCT4 (FIG. 25A) and SSEA1 (FIG. 25B). FIG. 25A: OCT4 (red color), DAPI (nuclei staining, blue color) and a merged image (“Merge”, with a double staining of red and blue colors showing pluripotent stem cells). FIG. 25B: SSEA1 (orange color), DAPI (nuclei staining, blue color) and a merged image (“Merge”, with a double staining of orange and blue colors showing pluripotent stem cells). The results show positive staining for OCT4 and SSEA1, demonstrating that the medium supplemented with PMSF at a concentration of 100 μM supports pluripotency of porcine PSCs for at least 5 passages while cultured on a 3D suspension culture. Magnification ×10.

FIGS. 26A-B are images depicting expression of TRA1-60 and Nanog in undifferentiated bovine PSCs (iBVN 1.4) which were cultured in a defined, serum-free culture medium (“IT1”) on a two-dimensional culture system. The defined, serum-free culture medium (“IT1”) is composed of: DMEM/F12 supplemented with bFGF (50 ng/ml), IL6RIL6 (100 μg/ml), lipid mixture 1%, insulin 0.43 μM, transferrin 0.0172 μM, BSA 0.5%, L-glutamine 4 μM, ascorbic acid 500 μg/ml and antibiotics (Penicillin: 50 U/ml and Streptomycin: 0.05 mg/ml). Bovine PSCs were cultured in 2D (on MEFs feeder cells) with the defined, serum-free culture media and following 3 passages in culture the cells were subjected to immunofluorescence analysis for the key pluripotency markers TRA1-60 (FIG. 26A) and Nanog (FIG. 26B). FIG. 26A: TRA1-60 (orange color), DAPI (nuclei staining, blue color) and a merged image (“Merge”, with a double staining of orange and blue colors showing pluripotent stem cells). FIG. 26B: Nanog (green color), DAPI (nuclei staining, blue color) and a merged image (“Merge”, with a double staining of green and blue colors showing pluripotent stem cells). The results show positive staining for TRA1-60 and Nanog, demonstrating that the defined, serum-free medium (“IT1” medium) supports pluripotency of bovine PSCs for at least 3 passages while cultured on a 2D culture system. Magnification ×10.

FIGS. 27A-B are images depicting expression of TRA1-60 and Nanog in undifferentiated bovine PSCs (iBVN 1.4) which were cultured on a two-dimensional culture system in a defined, serum-free medium (“IT2”). The defined culture medium (IT2 medium) is composed of DMEM/F12 supplemented with bFGF (50 ng/ml), IL6RIL6 chimera (100 μg/ml), lipid mixture 1% (v/v), insulin 1.57 μM, transferrin 0.055 μM, bovine serum albumin (BSA) 0.5% (v/v), ascorbic acid 500 μg/ml, L-glutamine 4 μM, and antibiotics (Penicillin: 50 U/ml and Streptomycin: 0.05 mg/ml). Bovine PSCs were cultured in 2D (on MEFs feeder cells) with the defined, serum-free culture medium (“IT2”) and following 3 passages in culture the cells were subjected to immunofluorescence analysis for the key pluripotency markers TRA1-60 (FIG. 27A) and Nanog (FIG. 27B). FIG. 27A: TRA1-60 (orange color), DAPI (nuclei staining, blue color) and a merged image (“Merge”, with a double staining of orange and blue colors showing pluripotent stem cells). FIG. 27B: Nanog (green color), DAPI (nuclei staining, blue color) and a merged image (“Merge”, with a double staining of green and blue colors showing pluripotent stem cells). The results show positive staining for TRA1-60 and Nanog, demonstrating that the defined, serum-free medium supports pluripotency of bovine PSCs for at least 3 passages while cultured on a 2D culture system. Magnification ×10.

FIG. 28 are images depicting expression of OCT4 in undifferentiated porcine PSCs (Psus 1) which were cultured in a CNTF and IL11 medium (with CNTF (1 ng/ml) and IL11 (1 ng/ml)) on a two-dimensional culture system. Porcine PSCs were cultured in 2D (on MEFs feeder cells) with a medium supplemented with CNTF and IL11 and following 5 passages in culture the cells were subjected to immunofluorescence analysis for the key pluripotency marker OCT4. Shown are images of OCT4 staining (red color), DAPI (nuclei staining, blue color) and a merged image (“Merge”, with a double staining of red and blue colors showing pluripotent stem cells). The results show positive staining for OCT4, demonstrating that a medium supplemented with CNTF and IL11 supports pluripotency of porcine PSCs for at least 5 passages while cultured on a 2D culture system. Magnification ×10.

FIGS. 29A-B are images depicting expression of OCT4 and SSEA1 in undifferentiated porcine PSCs (Psus 1) which were cultured in a PMSF medium with a concentration of 100 μM PMSF on a two-dimensional culture system. Porcine PSCs were cultured in 2D (on MEFs feeder cells) with a culture media supplemented with PMSF at a concentration of 100 μM and following 5 passages in culture the cells were subjected to immunofluorescence analysis for the key pluripotency markers OCT4 (FIG. 29A) and SSEA1 (FIG. 29B). FIG. 29A: OCT4 (red color), DAPI (nuclei staining, blue color) and a merged image (“Merge”, with a double staining of red and blue colors showing pluripotent stem cells). FIG. 29B: SSEA1 (orange color), DAPI (nuclei staining, blue color) and a merged image (“Merge”, with a double staining of orange and blue colors showing pluripotent stem cells). The results show positive staining for OCT4 and SSEA1, demonstrating that a medium supplemented with PMSF supports pluripotency of porcine PSCs for at least 5 passages while cultured on a 2D culture system. Magnification ×10.

FIGS. 30A-B are images depicting expression of SSEA1 and Nanog in undifferentiated porcine PSCs (Psus 1) which were cultured in a defined, serum-free culture medium (“IT1” medium) on a two-dimensional culture system. Porcine PSCs were cultured in 2D (on MEFs feeder cells) with the defined, serum-free culture medium and following 5 passages in culture the cells were subjected to immunofluorescence analysis for the key pluripotency markers SSEA1 (FIG. 30A) and Nanog (FIG. 30B). FIG. 30A: SSEA1 (orange color), DAPI (nuclei staining, blue color) and a merged image (“Merge”, with a double staining of orange and blue colors showing pluripotent stem cells). FIG. 30B: Nanog (green color), DAPI (nuclei staining, blue color) and a merged image (“Merge”, with a double staining of green and blue colors showing pluripotent stem cells). The results show positive staining for SSEA1 and Nanog, demonstrating that the defined, serum-free medium (“IT1” medium) supports pluripotency of porcine PSCs for at least 5 passages while cultured on a 2D culture system. Magnification ×20.

FIG. 31 shows images depicting expression of SSEA1 in undifferentiated porcine PSCs (Psus 1) which were cultured in a defined, serum-free culture medium (“IT2” medium) on a two-dimensional culture system. Porcine PSCs were cultured in 2D (on MEFs feeder cells) with the defined, serum-free culture medium and following 5 passages the cells were subjected to immunofluorescence analysis for the key pluripotency marker SSEA1. Shown are images of SSEA1 (orange color), DAPI (nuclei staining, blue color) and a merged image (“Merge”, with a double staining of orange and blue colors showing pluripotent stem cells). The results show positive staining for SSEA1, demonstrating that the defined, serum-free medium (“IT2” medium) supports pluripotency of porcine PSCs for at least 5 passages while cultured on a 2D culture system. Magnification ×20.

FIGS. 32A-B are images depicting expression of TRA1-60 and TRA1-81 in undifferentiated bovine PSCs (iBVN 1.4) which were cultured in a defined, serum-free culture medium (“IT1” medium) on a two-dimensional culture system. Bovine PSCs were cultured in 2D (on MEFs feeder cells) with the defined, serum-free culture medium and following 5 passages the cells were subjected to immunofluorescence analysis for the key pluripotency markers TRA1-60 (FIG. 32A) and TRA1-81 (FIG. 32B). FIG. 32A: TRA1-60 (orange color), DAPI (nuclei staining, blue color) and a merged image (“Merge”, with a double staining of orange and blue colors showing pluripotent stem cells). FIG. 32B: TRA1-81 (green color), DAPI (nuclei staining, blue color) and a merged image (“Merge”, with a double staining of green and blue colors showing pluripotent stem cells). The results show positive staining for TRA1-60 and TRA1-81, demonstrating that the defined, serum-free medium (“IT1” medium) supports pluripotency of bovine PSCs for at least 5 passages while cultured on a 2D culture system. Magnification TRA1-60 (×20) and TRA1-81 (×10).

FIGS. 33A-B are images depicting expression of TRA1-60 and TRA1-81 in undifferentiated bovine PSCs (iBVN 1.4) which were cultured in a defined, serum-free culture medium (“IT2” medium) on a two-dimensional culture system. Bovine PSCs were cultured in 2D (on MEFs feeder cells) with the defined, serum-free culture medium and following 5 passages in culture the cells were subjected to immunofluorescence analysis for the key pluripotency markers TRA1-60 (FIG. 33A) and TRA1-81 (FIG. 33B). FIG. 33A: TRA1-60 (orange color), DAPI (nuclei staining, blue color) and a merged image (“Merge”, with a double staining of orange and blue colors showing pluripotent stem cells). FIG. 33B: TRA1-81 (green color), DAPI (nuclei staining, blue color) and a merged image (“Merge”, with a double staining of green and blue colors showing pluripotent stem cells). The results show positive staining for TRA1-60 and TRA1-81, demonstrating that the defined, serum-free medium (“IT2” medium) supports pluripotency of bovine PSCs for at least 5 passages while cultured on a 2D culture system. Magnification TRA1-60 (×10) and TRA1-81 (×20).

FIGS. 34A-C are images depicting expression of TRA1-60, TRA1-81 and Nanog in undifferentiated bovine PSCs (iBVN 1.4) which were cultured in a culture medium supplemented with PMSF at a concentration of 70 μM on a two-dimensional culture system. Bovine PSCs were cultured in 2D (on MEFs feeder cells) with the PMSF culture medium and following 6 passages in culture the cells were subjected to immunofluorescence analysis for the key pluripotency markers TRA1-60 (FIG. 34A), TRA1-81 (FIG. 34B) and Nanog (FIG. 34C). FIG. 34A: TRA1-60 (orange color), DAPI (nuclei staining, blue color) and a merged image (“Merge”, with a double staining of orange and blue colors showing pluripotent stem cells). FIG. 34B: TRA1-81 (green color), DAPI (nuclei staining, blue color) and a merged image (“Merge”, with a double staining of green and blue colors showing pluripotent stem cells). FIG. 34C: Nanog (green color), DAPI (nuclei staining, blue color) and a merged image (“Merge”, with a double staining of green and blue colors showing pluripotent stem cells). The results show positive staining for TRA1-60, TRA1-81 and Nanog, demonstrating that the medium supplemented with PMSF at a concentration of 70 μM supports pluripotency of bovine PSCs for at least 6 passages while cultured on a 2D culture system. Magnification ×20.

### DESCRIPTION OF SPECIFIC EMBODIMENTS OF THE INVENTION

The present invention, in some embodiments thereof, relates to defined culture media suitable for expansion of mammalian pluripotent stem cells (such as mammalian livestock pluripotent stem cells) in an undifferentiated state and, more particularly, but not exclusively, to cell cultures comprising cells and the defined culture media, and methods of expanding mammalian pluripotent stem cells using the defined culture media.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The Examples section which follows shows that culture media supplemented with low concentrations of serum replacement (Insulin, transferrin, albumin, ascorbic acid and fatty acids) can support the undifferentiated growth of mammalian pluripotent stem cells such as livestock pluripotent stem cells. The defined serum-free culture media identified by the present inventor can support efficient growth of mammalian pluripotent stem cells (e.g., bovine or porcine PSCs) while using feeder layers, feeder layer-free and carrier free suspension cultures.

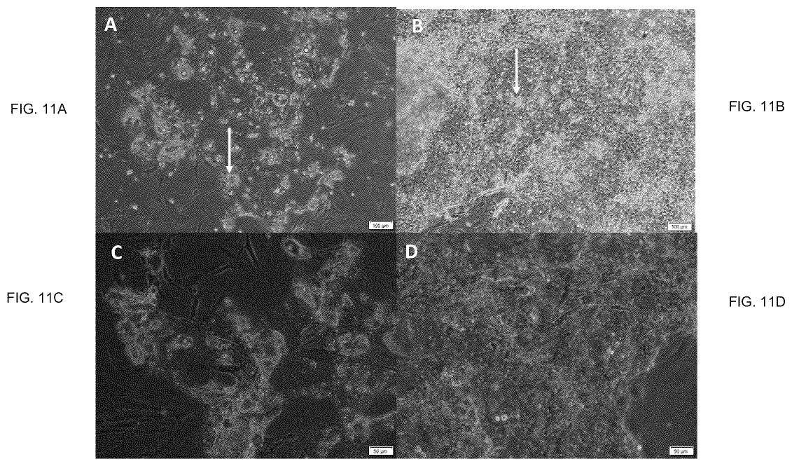

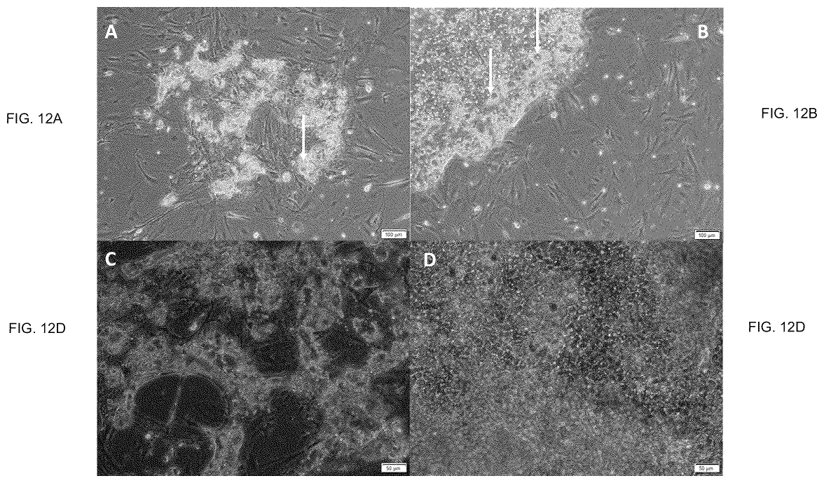

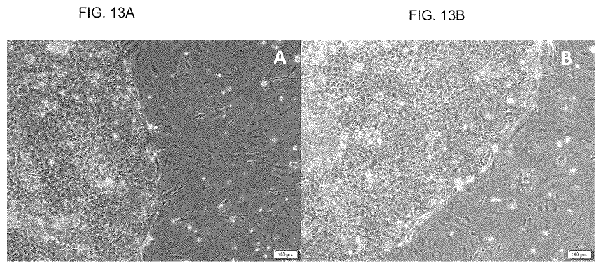

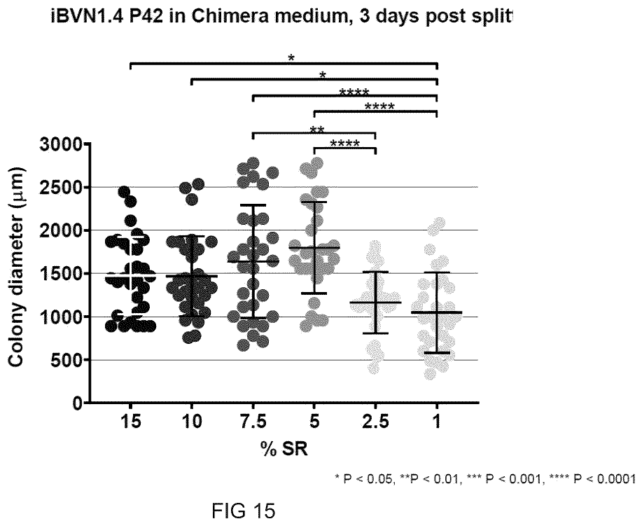

Example 4 of the Example section which follows and FIG. 15 show that the colony diameter of PPSCs cultured in as low as 1-2.5% (volume/volume) KoSR is smaller than that of cells cultured with 5% (v/v) KoSR or with higher concentrations of 7.5% (v/v), 10% (v/v) or 15% (v/v) KoSR, indicating a somewhat slower growth rate of colonies during the first 1-7 passages. On the other hand, at concentrations of 1-2.5% (v/v) KoSR there is no significant background differentiation of the PPSCs (described in Example 1 above and in FIGS. 13A-B, less than 3% background differentiation) and at a concentration of 5% KoSR there is about 5% background differentiation to adipocyte cells. In contrast, at a concentration of 10% KoSR there is about 10% background differentiation to adipocyte cells (FIGS. 12A-D); and at a concentration of 15% KoSR there is about 15-20% background differentiation to adipocyte cells (FIGS. 11A-D), thus these results show that increasing the concentration of KoSR from 5% (v/v) to 10% (v/v) or 15% (v/v) results in increasing of background differentiation.

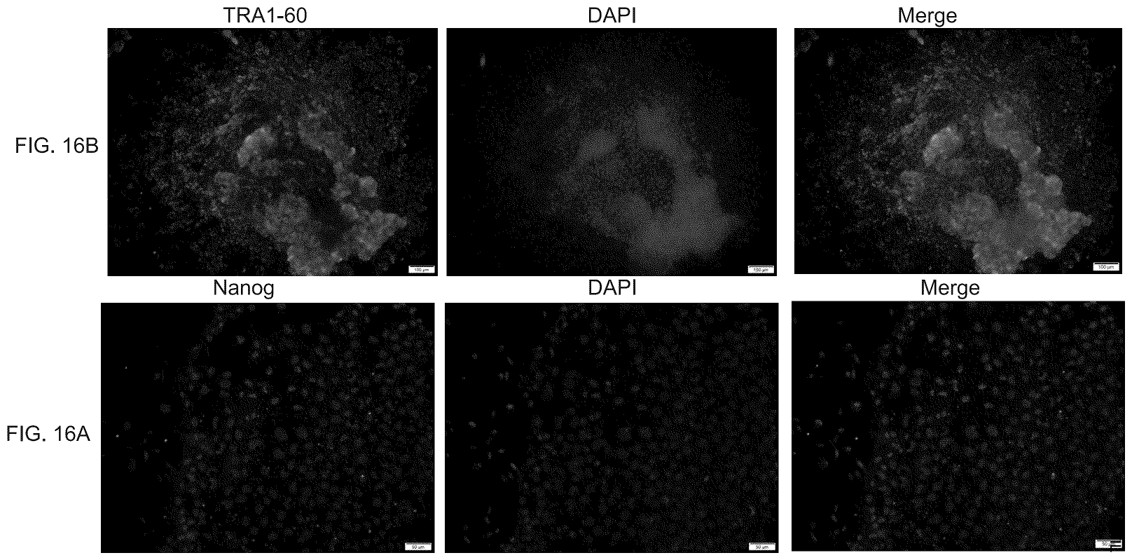

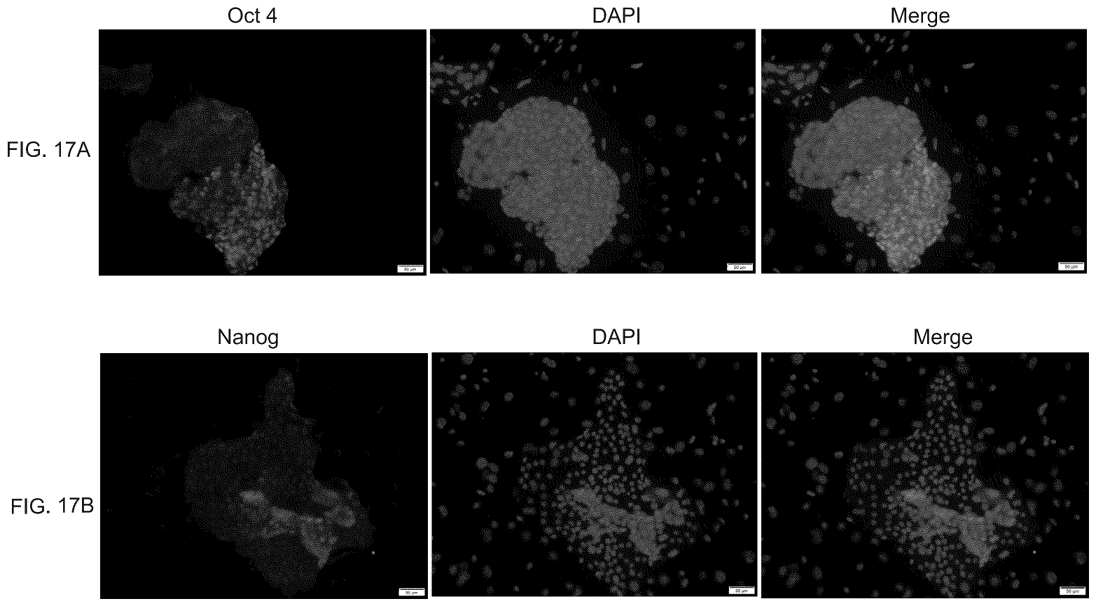

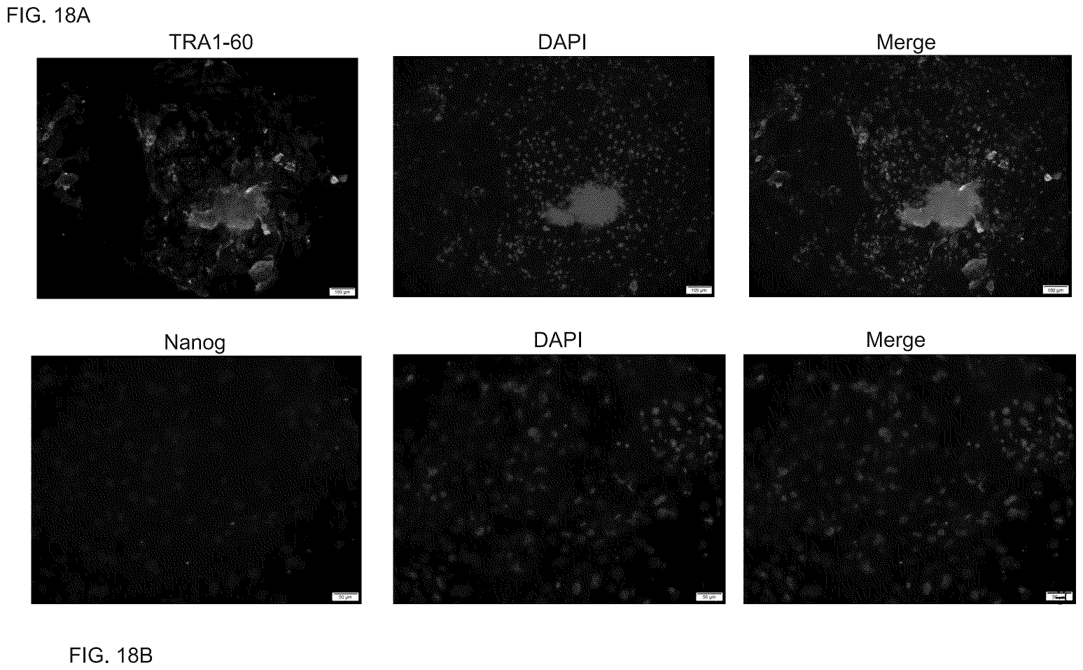

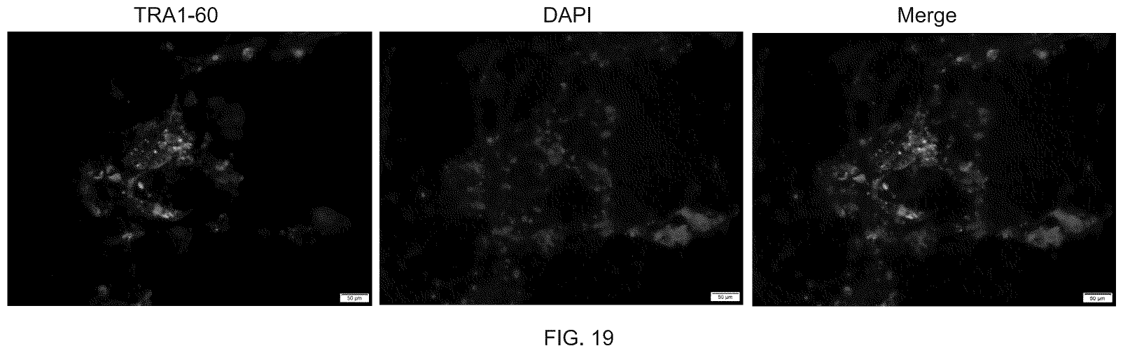

Examples 5 and 6 of the Examples section which follows demonstrate the ability of culture media which comprise the low concentrations of serum replacement, e.g., 5% (v/v) of KoSR, supplemented with gp130 agonists such as CNTF and IL11 (FIGS. 16A-B, 17A-C, 22, 23A-D, 24A-B and 28), or with a protease inhibitor PMSF (FIGS. 18A-B, 19, 20A-C, 21A-B, 25A-B, 29A-B and 34A-C) to maintain bovine or porcine iPSCs in a pluripotent and undifferentiated state when cultured on feeder cell layers (two-dimensional culture systems) or in suspension cultures without substrate adherence (three-dimensional culture systems).

The present inventors have uncovered that serum replacement which comprises insulin and transferrin, but not selenium, can be used in a culture medium, along with a differentiation inhibitory factor(s), to maintain mammalian livestock pluripotent stem cells in an undifferentiated state when cultured in a two-dimensional or three-dimensional culture system.

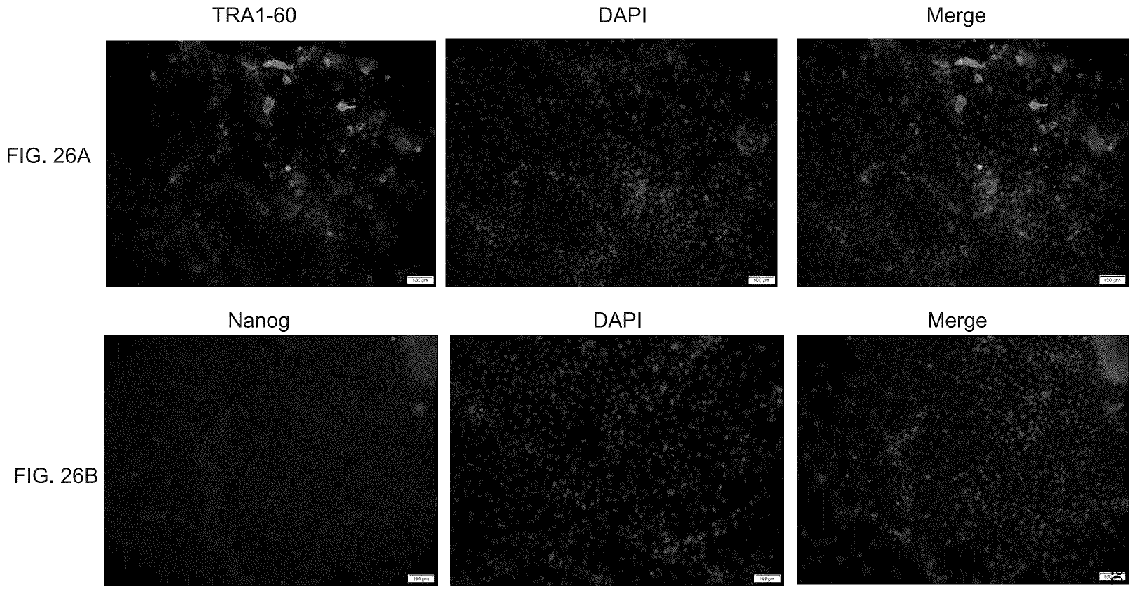

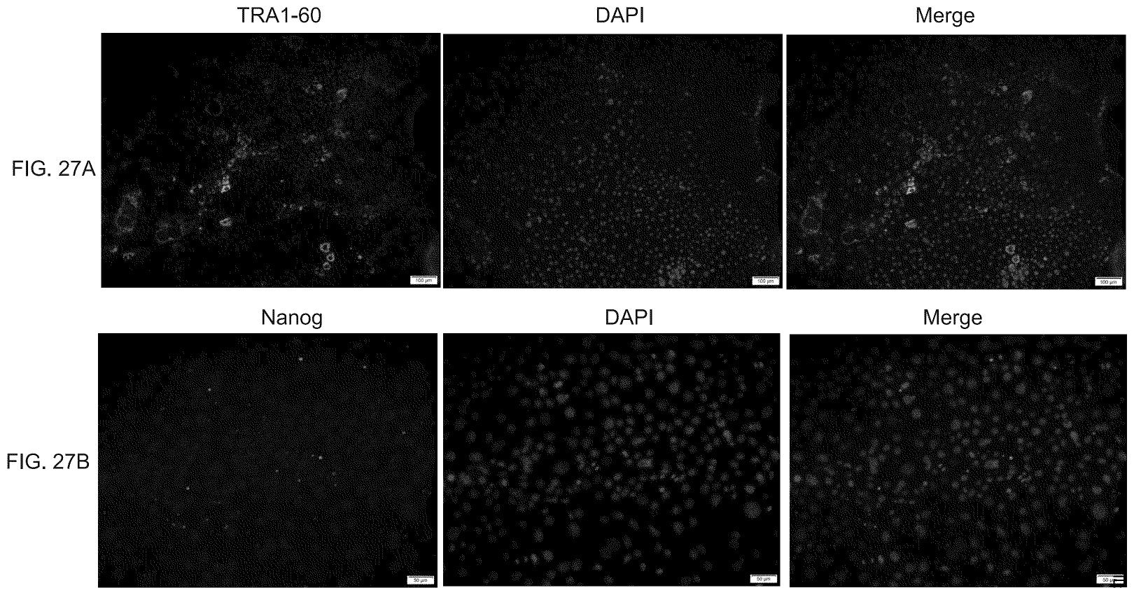

Example 7 of the Example section which follows shows that chemically defined culture media which comprise insulin and transferrin and are devoid of selenium, an effective concentration of at least one differentiation inhibiting agent (e.g., bFGF, and IL6RIL6 chimera), and optionally also ascorbic acid, are capable of maintaining the undifferentiated growth of mammalian livestock pluripotent stem cells for at least 3 or 5 passages (FIGS. 26A-B, 27A-B, 30A-B, 31, 32A-B and 33A-B).

According to an aspect of some embodiments of the invention there is provided a defined serum-free culture medium comprising a basal medium, serum replacement and an effective concentration of at least one differentiation inhibiting agent, wherein the defined culture medium is capable of maintaining mammalian livestock pluripotent stem cells in an undifferentiated state for at least 5 passages in culture, wherein the basal medium is selected suitable for maintaining pluripotent stem cells in an undifferentiated state, wherein the serum replacement comprises insulin and transferrin, and wherein the serum replacement is devoid of selenium.

According to some embodiments of the invention, the insulin is provided at a concentration in a range of 0.34×10⁻³ mM to 1.88×10⁻³ mM, and wherein the transferrin is provided at a concentration in a range of 0.137×10⁻⁴ mM to 0.66×10⁻⁴ mM.

According to an aspect of some embodiments of the invention there is provided a defined serum-free culture medium comprising a basal medium, serum replacement and an effective concentration of at least one differentiation inhibiting agent, wherein the defined culture medium is capable of maintaining mammalian livestock pluripotent stem cells in an undifferentiated state for at least 5 passages in culture, wherein the basal medium is selected suitable for maintaining pluripotent stem cells in an undifferentiated state, wherein the serum replacement comprises insulin and transferrin, wherein the insulin is provided at a concentration in a range of 0.34×10⁻³ mM to 1.88×10⁻³ mM, and wherein the transferrin is provided at a concentration in a range of 0.137×10⁻⁴ mM to 0.66×10⁻⁴ mM.

As used herein the phrase “serum-free” refers to being devoid of a human or an animal serum.

According to some embodiments of the invention, the serum-free culture medium does not comprise serum or portions thereof.

According to some embodiments of the invention, the serum-free culture medium does not comprise serum or portions thereof with the proviso that the serum-free culture medium may comprise bovine serum albumin.

As used herein the phrase “culture medium” refers to a liquid substance used to support the growth of cells. The culture medium used by the invention according to some embodiments can be a water-based medium which includes a combination of substances such as salts, nutrients, minerals, vitamins, amino acids, nucleic acids, proteins such as cytokines, growth factors and hormones, all of which are needed for cell proliferation and/or differentiation.

The culture medium of some embodiments of the invention comprises a basal medium. The basal medium is a synthetic culture medium which can be supplemented with additives such as amino acid(s) (e.g., L-Glutamin, non-essential amino acids (NEAA)), β-mercaptoethanol and/or antibiotics. It should be noted that some synthetic culture media already include non-essential amino acids (NEAA), lipids and/or albumin.

For example, a culture medium according to an aspect of some embodiments of the invention can include a synthetic tissue culture basal medium such as the Dulbecco's Modified Eagle's Medium (DMEM, e.g., available for example from Gibco-Invitrogen Corporation products, Grand Island, NY, USA), DMEM/F12 (e.g., available for example from Biological Industries, Biet HaEmek, Israel), Ko-DMEM (e.g., available for example from Gibco-Invitrogen Corporation products, Grand Island, NY, USA), or Eagle's Minimum Essential Medium (EMEM, e.g., available for example from Gibco-Invitrogen Corporation products, Grand Island, NY, USA) supplemented with the necessary additives as is further described hereinunder. The concentration of the basal medium depends on the concentration of the other medium ingredients such as the serum replacement as discussed below.

According to some embodiments of the invention, the basal medium is not RPMI1640.

According to some embodiments of the invention, the basal medium is selected from the group consisting of KO-DMEM, DMEM/F12 and DMEM.

According to some embodiments of the invention, the basal medium is selected from the group consisting of KO-DMEM and DMEM/F12.

According to some embodiments of the invention, the basal medium is KO-DMEM.

According to some embodiments of the invention, the basal medium is DMEM/F12.

According to some embodiments of the invention, the basal medium is provided at a concentration in a range of 94-96%.

A “defined” culture medium as used herein refers to a chemically-defined culture medium manufactured from known components at specific concentrations. For example, a defined culture medium is a non-conditioned culture medium.

Conditioned medium is the growth medium of a monolayer cell culture (i.e., feeder cells) present following a certain culturing period. The conditioned medium includes growth factors and cytokines secreted by the monolayer cells in the culture.

Conditioned medium can be collected from a variety of cells forming monolayers in culture. Examples include mouse embryonic fibroblasts (MEF) conditioned medium, foreskin conditioned medium, human embryonic fibroblasts conditioned medium, human fallopian epithelial cells conditioned medium, and the like.

As used herein the phrase “serum replacement” refers to a defined formulation, which substitutes the function of serum by providing pluripotent stem cells with components needed for growth and viability.

According to some embodiments of the invention the concentration of insulin in the serum replacement of the defined culture medium is at least 0.34×10⁻³ mM and not exceeding 1.88×10⁻³ mM, e.g., 0.43×10⁻³ mM and not exceeding 1.57×10⁻³ mM, e.g., least 0.43×10⁻³ mM and not exceeding 1.0×10⁻³ mM, e.g., at least 0.78×10⁻³ mM and not exceeding 1.55×10⁻³ mM, e.g., at least 0.78×10⁻³ mM and not exceeding 1.53×10⁻³ mM, e.g., at least 0.78×10⁻³ mM and not exceeding 1.51×10⁻³ mM, e.g., at least 0.78×10⁻³ mM and not exceeding 1.50×10⁻³ mM, e.g., at least 0.78×10⁻³ mM and not exceeding 1.4×10⁻³ mM, e.g., at least 0.78×10⁻³ mM and not exceeding 1.3×10⁻³ mM, e.g., at least 0.78×10⁻³ mM and not exceeding 1.1×10⁻³ mM, e.g., at least 0.78×10⁻³ mM and not exceeding 1.0×10⁻³ mM, e.g., about 0.43×10⁻³ mM, e.g., about 1.57×10⁻³ mM.

According to some embodiments of the invention the concentration of insulin in the serum replacement of the defined culture medium is at least 0.34×10⁻³ mM and not exceeding 1.57×10⁻³ mM, e.g., at least 0.34×10⁻³ mM and not exceeding 1.0×10⁻³ mM, at least 0.34×10⁻³ mM and not exceeding 0.87×10⁻³ mM, at least 0.34×10⁻³ mM and not exceeding 0.87×10⁻³ mM, at least 0.34×10⁻³ mM and not exceeding 0.528×10⁻³ mM, at least 0.43×10⁻³ mM and not exceeding 0.528×10⁻³ mM, at least 0.34×10⁻³ mM and not exceeding 0.516×10⁻³ mM, at least 0.34×10⁻³ mM and not exceeding 0.473×10⁻³ mM, at least 0.4×10⁻³ mM and not exceeding 0.65×10⁻³ mM, e.g., about 0.58×10⁻³ mM.

According to some embodiments of the invention the concentration of insulin in the serum replacement of the defined culture medium is at least 0.8×10⁻³ mM and not exceeding 1.50×10⁻³ mM, e.g., at least 0.9×10⁻³ mM and not exceeding 1.50×10⁻³ mM, e.g., at least 1.0×10⁻³ mM and not exceeding 1.5×10⁻³ mM, e.g., at least 1.1×10⁻³ mM and not exceeding 1.5×10⁻³ mM, e.g., at least 1.2×10⁻³ mM and not exceeding 1.5×10⁻³ mM.

According to some embodiments of the invention the concentration of transferrin in the serum replacement of the defined culture medium is at least 0.137×10⁻⁴ mM and not exceeding 0.66×10⁻⁴ mM, at least 0.172×10⁻⁴ mM and not exceeding 0.55×10⁻⁴ mM, e.g., at least 0.172×10⁻⁴ mM and not exceeding 0.34×10⁻⁴ mM, e.g., at least 0.27×10⁻⁴ mM and not exceeding 0.54×10⁻⁴ mM, e.g., at least 0.27×10⁻⁴ mM and not exceeding 0.52×10⁻⁴ mM, e.g., at least 0.27×10⁻⁴ mM and not exceeding 0.5×10⁻⁴ mM, e.g., at least 0.27×10⁻⁴ mM and not exceeding 0.49×10⁻⁴ mM, e.g., at least 0.27×10⁻⁴ mM and not exceeding 0.48×10⁻⁴ mM, e.g., at least 0.27×10⁻⁴ mM and not exceeding 0.47×10⁻⁴ mM, e.g., at least 0.27×10⁻⁴ mM and not exceeding 0.46×10⁻⁴ mM, e.g., at least 0.27×10⁻⁴ mM and not exceeding 0.45×10⁻⁴ mM, e.g., at least 0.27×10⁻⁴ mM and not exceeding 0.44×10⁻⁴ mM, e.g., at least 0.27×10⁻⁴ mM and not exceeding 0.43×10⁻⁴ mM, e.g., at least 0.27×10⁻⁴ mM and not exceeding 0.42×10⁻⁴ mM, e.g., at least 0.27×10⁻⁴ mM and not exceeding 0.41×10⁻⁴ mM, e.g., at least 0.27×10⁻⁴ mM and not exceeding 0.4×10⁻⁴ mM, e.g., at least 0.27×10⁻⁴ mM and not exceeding 0.39×10⁻⁴ mM, e.g., at least 0.27×10⁻⁴ mM and not exceeding 0.38×10⁻⁴ mM, e.g., at least 0.27×10⁻⁴ mM and not exceeding 0.37×10⁻⁴ mM, e.g., at least 0.27×10⁻⁴ mM and not exceeding 0.36×10⁻⁴ mM, e.g., at least 0.27×10⁻⁴ mM and not exceeding 0.35×10⁻⁴ mM, e.g., at least 0.27×10⁻⁴ mM and not exceeding 0.34×10⁻⁴ mM, e.g., about 0.172×10⁻⁴ mM, e.g., about 0.55×10⁻⁴ mM.

According to some embodiments of the invention the concentration of transferrin in the serum replacement of the defined culture medium is at least 0.27×10⁻⁴ mM and not exceeding 0.54×10⁻⁴ mM, e.g., at least 0.28×10⁻⁴ mM and not exceeding 0.54×10⁻⁴ mM, e.g., at least 0.29×10⁻⁴ mM and not exceeding 0.54×10⁻⁴ mM, e.g., at least 0.3×10⁻⁴ mM and not exceeding 0.54×10⁻⁴ mM, e.g., at least 0.31×10⁻⁴ mM and not exceeding 0.54×10⁻⁴ mM, e.g., at least 0.32×10⁻⁴ mM and not exceeding 0.54×10⁻⁴ mM, e.g., at least 0.33×10⁻⁴ mM and not exceeding 0.54×10⁻⁴ mM, e.g., at least 0.34×10⁻⁴ mM and not exceeding 0.5×10⁻⁴ mM, e.g., at least 0.35×10⁻⁴ mM and not exceeding 0.45×10⁻⁴ mM, e.g., at least 0.37×10⁻⁴ mM and not exceeding 0.4×10⁻⁴ mM.

According to some embodiments of the invention the defined culture medium according to some embodiments of the invention does not comprise selenium.

It should be noted that selenium is often available as a sodium selenite salt.

According to some embodiments of the invention the defined culture medium according to some embodiments of the invention may comprise trace amounts of selenium, e.g., an amount not exceeding 2.11×10⁻⁶ mM, e.g., an amount not exceeding 2.11×10⁻⁷ mM, e.g., an amount not exceeding 2.11×10⁻⁸ mM, e.g., an amount not exceeding 2.11×10⁻⁹ mM, e.g., an amount not exceeding 2.11×10⁻¹⁰ mM, e.g., an amount not exceeding 2.11×10⁻¹¹ mM, e.g., an amount not exceeding 2.11×10⁻¹² mM.

According to some embodiments of the invention the serum replacement further comprises ascorbic acid at a concentration in a range of 125-170 ng/ml.

According to some embodiments of the invention the serum replacement further comprises ascorbic acid at a concentration in a range of 8-17 micrograms/milliliter.

According to some embodiments of the invention the serum replacement further comprises ascorbic acid at a concentration in a range of 10-15 micrograms/milliliter According to some embodiments of the invention the serum replacement further comprises ascorbic acid at a concentration in a range of 11.125-14.8 micrograms/milliliter.

According to some embodiments of the invention the serum replacement further comprises bovine serum albumin at a concentration in a range of 0.4% to 0.7% (volume/volume (v/v), e.g., 0.45% to 0.7% (v/v), 0.5% to 0.66% volume/volume (v/v), e.g., in a range of 0.51%-0.65% v/v, e.g., in a range of 0.52%-0.64% v/v, e.g., in a range of 0.53%-0.63% v/v, e.g., in a range of 0.54%-0.62% v/v, e.g., in a range of 0.55%-0.61% v/v, e.g., in a range of 0.56%-0.6% v/v, e.g., in a range of 0.57%-0.6% v/v, e.g., about 0.5% v/v.

According to some embodiments of the invention the serum replacement further comprises a lipid mixture.

As used herein the phrase “lipid mixture” refers to a defined (e.g., chemically defined) lipid composition needed for culturing the pluripotent stem cells in an undifferentiated state.

It should be noted that the lipid mixture is usually added to a culture medium which is devoid of serum.

In addition, since some commercially available serum replacement formulations such as GIBCO™ Knockout™ Serum Replacement already include lipids, the lipid mixture is usually added to a medium which does not include the GIBCO™ Knockout™ Serum Replacement.

A non-limiting example of a commercially available lipid mixture, which can be used in the culture medium of some embodiments of the invention, is the Chemically Define Lipid Concentrate (e.g., available from Invitrogen, Catalogue No. 11905-031).

According to some embodiments of the invention, the lipid mixture comprised in the serum replacement of some embodiments of the invention is the Chemically Define Lipid Concentrate.

According to some embodiments of the invention, the concentration of the lipid mixture in the culture medium is from about 0.5% [volume/volume (v/v)] to about 1.2% v/v, e.g., from about 0.6% v/v to about 1% v/v, e.g., from about 0.7% v/v to about 1% v/v, e.g., from about 0.8% v/v to about 1% v/v, e.g., from about 0.9% v/v to about 1% v/v, e.g., about 1% v/v.

According to some embodiments of the invention the serum replacement included in the defined culture medium of some embodiments of the invention comprises insulin (at a concentration range of 0.34×10⁻³ mM to 1.88×10⁻³ mM), transferrin (at a concentration range of 0.137×10⁻⁴ mM to 0.66×10⁻⁴ mM, and a lipid mixture at concentration of 0.5% [volume/volume (v/v)] to 1.2% v/v, wherein the serum replacement is devoid of selenium.

According to some embodiments of the invention the serum replacement included in the defined culture medium of some embodiments of the invention comprises insulin (at a concentration range of 0.34×10⁻³ mM to 1.88×10⁻³ mM), transferrin (at a concentration range of 0.137×10⁻⁴ mM to 0.66×10⁻⁴ mM, a lipid mixture at concentration of 0.5% [volume/volume (v/v)] to 1.2% v/v, and bovine serum albumin (BSA) at a concentration range of BSA 0.4% (v/v) to 0.7% (v/v), wherein the serum replacement is devoid of selenium.

Following are non-limiting exemplary serum replacement formulations which comprise insulin and transferrin but not selenium, and which can be part of the defined culture media of some embodiments of the invention:
(i) Insulin 0.387-0.473 μM, Transferrin 0.0155-0.0189 μM, lipid mixture 0.9-1.1% volume/volume, bovine serum albumin 0.45-0.55% v/v, and optionally ascorbic acid at a concentration of 10-15 μg/ml.
(ii) Insulin 1.413-1.727 μM, Transferrin 0.0495-0.0605 μM, lipid mixture 0.9-1.1% volume/volume, bovine serum albumin 0.45-0.55% v/v, and optionally ascorbic acid at a concentration of 10-15 μg/ml.
(iii) Insulin 0.387-0.473 μM, Transferrin 0.0155-0.0189 μM, lipid mixture 0.9-1.1% volume/volume, bovine serum albumin 0.45-0.55% v/v, and optionally ascorbic acid at a concentration of 125-170 ng/ml.
(iv) Insulin 1.413-1.727 μM, Transferrin 0.0495-0.0605 μM, lipid mixture 0.9-1.1% volume/volume, bovine serum albumin 0.45-0.55% v/v, and optionally ascorbic acid at a concentration of 125-170 ng/ml.

According to some embodiments of the invention the defined culture medium according to some embodiments of the invention further comprises selenium.

According to some embodiments of the invention the concentration of selenium in the defined culture medium does not exceed 2.23×10⁻⁴ gram per liter or 5.9×10⁻⁵ mM.

According to some embodiments of the invention the concentration of selenium in the defined culture medium is in the range of 2.11×10⁻⁵ mM to 5.9×10⁻⁵ mM, e.g., in the range of 4.4×10⁻⁵ mM to 5.9×10⁻⁵ mM.

According to some embodiments of the invention the serum replacement further comprises a lipid selected from the group consisting of: Linoleic Acid at a concentration in a range of 0.47-0.63×10⁻⁴ mM, Lipoic Acid at a concentration in a range of 1-1.33×10⁻⁴ mM, Arachidonic Acid at a concentration in a range of 0.32-0.43×10⁻⁵ mM, Cholesterol at a concentration in a range of 0.28-0.37×10⁻³ mM, DL-alpha tocopherol-acetate at a concentration in a range of 0.72-0.96×10⁻³ mM, Linolenic Acid at a concentration in a range of 1.74-2.33×10⁻⁵ mM Myristic Acid at a concentration in a range of 2.14-2.86×10⁻⁵ mM, Oleic Acid at a concentration in a range of 1.73-2.31×10⁻⁵ mM, Palmitic Acid at a concentration in a range of 1.91-2.55×10⁻⁵ mM, Palmitoleic acid at a concentration in a range of 1.92-2.571×10⁻⁵ mM, and Stearic Acid at a concentration in a range of 1.72-2.29×10⁻⁵ mM.

According to some embodiments of the invention the serum replacement used in the defined culture medium of some embodiments of the invention comprises Insulin (at a concentration range of 0.34×10⁻³ mM to 1.88×10⁻³ mM), transferrin (at a concentration range of 0.137×10⁻⁴ mM to 0.66×10⁻⁴ mM), Selenium (at a concentration range of 2.11×10⁻⁵ mM to 5.9×10⁻⁵ mM) and a lipid mixture (at a concentration of 0.5-1.2% (v/v)).

According to some embodiments of the invention the serum replacement used in the defined culture medium of some embodiments of the invention comprises Insulin (at a concentration range of 0.34×10⁻³ mM to 1.88×10⁻³ mM), transferrin (at a concentration range of 0.137×10⁻⁴ mM to 0.66×10⁻⁴ mM), Selenium (at a concentration range of 2.11×10⁻⁵ mM to 5.9×10⁻⁵ mM), a lipid mixture (at a concentration of 0.5-1.2% (v/v)) and bovine serum albumin (BSA) at a concentration range of BSA 0.4% (v/v) to 0.7% (v/v).

According to some embodiments of the invention the serum replacement used in the defined culture medium of some embodiments of the invention comprises Insulin (at a concentration range of 0.34×10⁻³ mM to 1.88×10⁻³ mM), transferrin (at a concentration range of 0.137×10⁻⁴ mM to 0.66×10⁻⁴ mM), Selenium (at a concentration range of 2.11×10⁻⁵ mM to 5.9×10⁻⁵ mM) and a fatty acid mix [including Linoleic Acid at a concentration in a range of 0.47-0.63×10⁻⁴ mM, Lipoic Acid at a concentration in a range of 1-1.33×10⁻⁴ mM, Arachidonic Acid at a concentration in a range of 0.32-0.43×10⁻⁵ mM, Cholesterol at a concentration in a range of 0.28-0.37×10⁻³ mM, DL-alpha tocopherol-acetate at a concentration in a range of 0.72-0.96×10⁻³ mM, Linolenic Acid at a concentration in a range of 1.74-2.33×10⁻⁵ mM Myristic Acid at a concentration in a range of 2.14-2.86×10⁻⁵ mM, Oleic Acid at a concentration in a range of 1.73-2.31×10⁻⁵ mM, Palmitic Acid at a concentration in a range of 1.91-2.55×10⁻⁵ mM, Palmitoleic acid at a concentration in a range of 1.92-2.571×10⁻⁵ mM, and Stearic Acid at a concentration in a range of 1.72-2.29×10⁻⁵ mM].

Various serum replacement formulations are known in the art and are commercially available.

For example, GIBCO™ Knockout™ Serum Replacement (Gibco-Invitrogen Corporation, Grand Island, NY USA; Catalogue No. 10828028) is a defined serum-free formulation optimized to grow and maintain undifferentiated ES cells in culture. It should be noted that the formulation of GIBCO™ Knockout™ Serum Replacement includes Albumax (Bovine serum albumin enriched with lipids) which is from an animal source (International Patent Publication No. WO 98/30679 to Price, P. J. et al). However, a recent publication by Crook et al., 2007 (Crook J M., et al., 2007, Cell Stem Cell, 1: 490-494) describes six clinical-grade hESC lines generated using FDA-approved clinical grade foreskin fibroblasts in cGMP-manufactured Knockout™ Serum Replacement (Invitrogen Corporation, USA, e.g., Catalogue No. 04-0095).

According to some embodiments of the invention, the concentration of GIBCO™ Knockout™ Serum Replacement in the defined culture medium is in the range of from about 1-10% volume/volume (v/v), e.g., at a concentration of 1-7.5% (v/v), e.g., 1-5% (v/v), e.g., 5-7.5% (v/v), e.g., 3%, 4%, 5%, 6%, 7% or 7.5% (v/v).

Another suitable commercially available serum replacement is the B27 supplement without vitamin A which is available from Gibco-Invitrogen, Corporation, Grand Island, NY USA, e.g., Catalogue No. 12587-010. The B27 supplement is a serum-free formulation which includes d-biotin, fatty acid free fraction V bovine serum albumin (BSA), catalase, L-carnitine HCl, corticosterone, ethanolamine HCl, D-galactose (Anhyd.), glutathione (reduced), recombinant human insulin, linoleic acid, linolenic acid, progesterone, putrescine-2-HCl, sodium selenite, superoxide dismutase, T-3/albumin complex, DL alpha-tocopherol and DL alpha tocopherol acetate.

For example, the formulation of SR3 (Sigma) is a xeno-free serum replacement.

According to some embodiments of the invention, the xeno-free serum replacement formulation SR3 (Sigma) is diluted in a 1 to 250 ratio in order to reach an X0.25 working concentration.

According to some embodiments of the invention the serum replacement is xeno-free.

The term “xeno” is a prefix based on the Greek word “Xenos”, i.e., a stranger. As used herein the phrase “xeno-free” refers to being devoid of any components which are derived from a xenos (i.e., not the same, a foreigner) species. Such components can be contaminants such as pathogens associated with (e.g., infecting) the xeno species, cellular components of the xeno species or a-cellular components (e.g., fluid) of the xeno species.

It should be noted that a composition comprising a combination of insulin, transferrin and selenium can be obtained from various sources. For example, a commercially available xeno-free serum replacement composition includes the premix of ITS (Insulin, Transferrin and Selenium) available from Invitrogen corporation (ITS, Invitrogen Corporation, e.g., Catalogue No. 51500-056).

According to some embodiments of the invention, the xeno-free serum replacement formulation ITS (Invitrogen Corporation), which is supplied as a ×100 solution, is diluted in a 1 to 250 ratio in order to reach an X0.25 working concentration.

According to some embodiments of the invention, the xeno-free serum replacement formulation ITS (Invitrogen Corporation), which is supplied as a ×100 solution, is diluted in a 1 to 330 ratio in order to reach an X0.33 working concentration.

As described, the defined culture medium of some embodiments of the invention comprises at least one differentiation inhibiting agent.

As used herein the phrase “differentiation inhibiting agent” refers to an agent which is capable of inhibiting differentiation of at least 50% of a population of pluripotent stem cells while cultured in vitro for at least 5 passages.

According to some embodiments of the invention, the differentiation inhibiting agent is capable of inhibiting differentiation of at least 50% or more of a population of pluripotent stem cells while cultured in vitro for at least 5 passages, e.g., for at least 10 passages, e.g., for at least 15 passages, e.g., for at least 20 passages, e.g., for at least 25 passages, e.g., for at least 30 passages, e.g., for at least 35 passages, e.g., for at least 40 passages.

According to some embodiments of the invention, the differentiation inhibiting agent is capable of inhibiting differentiation of at least 55%, e.g., at least 60%, e.g., at least 65%, e.g., at least 70%, e.g., at least 75%, e.g., at least 80%, e.g., at least 85%, e.g., at least 90%, e.g., at least 95% or more of a population of pluripotent stem cells while cultured in vitro for at least 5 passages.

According to some embodiments of the invention, the differentiation inhibiting agent is capable of inhibiting differentiation of at least 80% of a population of pluripotent stem cells while cultured in vitro for at least 5 passages, e.g., for at least 10 passages, e.g., for at least 15 passages, e.g., for at least 20 passages, e.g., for at least 25 passages, e.g., for at least 30 passages, e.g., for at least 35 passages, e.g., for at least 40 passages.

According to some embodiments of the invention, the differentiation inhibiting agent inhibits differentiation of the pluripotent stem cells when cultured in vitro in a suspension culture.

According to some embodiments of the invention, the differentiation inhibiting agent inhibits differentiation of the pluripotent stem cells when cultured in vitro in a feeder-free culture system.

According to some embodiments of the invention, the differentiation inhibiting agent inhibits differentiation of the pluripotent stem cells when cultured in vitro on feeder layers.

According to some embodiments of the invention, the differentiation inhibiting agent is capable of maintaining mammalian livestock pluripotent stem cells in an undifferentiated states for at least 6, 7, 8, 9, 10, 11, 12, 13, 14, 15, 16, 17, 18, 19, 20 or more passages in culture.

According to some embodiments of the invention, the at least one differentiation inhibiting agent is a growth factor, a cytokine, a small molecule, or a combination thereof, wherein the effective concentration of the at least one differentiation inhibiting agent is capable of maintaining the mammalian pluripotent stem cells (e.g., livestock pluripotent stem cells) in an undifferentiated states for at least 5 passages in culture.

According to some embodiments of the invention, the growth factor is basic fibroblast growth factor (bFGF).

As used herein the term “basic fibroblast growth factor (bFGF)” refers to a polypeptide of the fibroblast growth factor (FGF) family, which binds heparin and possesses broad mitogenic and angiogenic activities. The mRNA for the BFGF gene contains multiple polyadenylation sites, and is alternatively translated from non-AUG (CUG) and AUG initiation codons, resulting in five different isoforms with distinct properties. The CUG-initiated isoforms are localized in the nucleus and are responsible for the intracrine effect, whereas, the AUG-initiated form is mostly cytosolic and is responsible for the paracrine and autocrine effects of this FGF.

The bFGF polypeptide (e.g., GenBank Accession No. NP_001997 (SEQ ID NO:1) can be obtained from various manufacturers such as Peprotech, R&D systems (e.g., Catalog Number: 233-FB), and Millipore.

According to some embodiments of the invention, the effective concentration of the bFGF in the defined culture medium of some embodiments of the invention is in a range of 4-130 ng/ml.

According to some embodiments of the invention, the effective concentration of the bFGF in the defined culture medium of some embodiments of the invention is about 50 ng/ml.

According to some embodiments of the invention, the effective concentration of the bFGF in the defined culture medium of some embodiments of the invention is between 30 ng/ml to 70 ng/ml.

According to some embodiments of the invention, the effective concentration of the bFGF in the defined culture medium of some embodiments of the invention is about 10 ng/ml.

According to some embodiments of the invention, the effective concentration of the bFGF in the defined culture medium of some embodiments of the invention is between 4 ng/ml to 15 ng/ml.

According to some embodiments of the invention, the effective concentration of the bFGF in the defined culture medium of some embodiments of the invention is about 100 ng/ml bFGF.

According to some embodiments of the invention, the effective concentration of the bFGF in the defined culture medium of some embodiments of the invention is between 70-130 ng/ml.

According to some embodiments of the invention, the at least one differentiation inhibiting agent is the IL6RIL6 chimera.

As used herein the term “IL6RIL6” refers to a chimeric polypeptide which comprises the soluble portion of interleukin-6 receptor (IL-6-R, e.g., the human IL-6-R as set forth by GenBank Accession No. AAH89410, SEQ ID NO:2) (e.g., a portion of the soluble IL6 receptors as set forth by amino acids 112-355 of GenBank Accession No. AAH89410, SEQ ID NO:3) and the interleukin-6 (IL6) (e.g., human IL-6 as set forth by GenBank Accession No. CAG29292, SEQ ID NO:4) or a biologically active fraction thereof (e.g., a receptor binding domain).

Preferably, the IL6RIL6 chimera used by the method according to this aspect of the present invention is capable of supporting the undifferentiated growth of mammalian pluripotent stem cells (e.g., mammalian livestock pluripotent stem cells), while maintaining their pluripotent capacity. It will be appreciated that when constructing the IL6RIL6 chimera the two functional portions (i.e., the IL6 and its receptor) can be directly fused (e.g., attached or translationally fused, i.e., encoded by a single open reading frame) to each other or conjugated (attached or translationally fused) via a suitable linker (e.g., a polypeptide linker). Preferably, the IL6RIL6 chimeric polypeptide exhibits a similar amount and pattern of glycosylation as the naturally occurring IL6 and IL6 receptor. For example, a suitable IL6RIL6 chimera is as set forth in SEQ ID NO:5 and in FIG. 11 of WO 99/02552 to Revel M., et al., which is fully incorporated herein by reference.

According to some embodiments of the invention, the IL6RIL6 chimera which is included in the defined culture medium is present at a concentration of at least 50 pg/ml (picograms per milliliter) and not exceeding 150 pg/ml, e.g., at least 75 pg/ml and not exceeding 150 pg/ml, preferably at least 80 pg/ml and not exceeding 150 pg/ml, preferably, at least 85 pg/ml and not exceeding 150 pg/ml, preferably, at least 90 pg/ml and not exceeding 150 pg/ml, e.g., about 100 pg/ml.

According to some embodiments of the invention, the effective concentration of the IL6RIL6 chimera is about 100 pg/ml.

According to some embodiments of the invention, the IL6RIL6 chimera which is included in the defined culture medium of some embodiments of the invention is present at a concentration of at least 50 ng/ml (nanograms per milliliter) and not exceeding 150 ng/ml, e.g., at least 75 ng/ml and not exceeding 150 ng/ml, preferably at least 80 ng/ml and not exceeding 150 ng/ml, preferably, at least 85 ng/ml and not exceeding 150 ng/ml, preferably, at least 90 ng/ml and not exceeding 150 ng/ml, e.g., about 100 ng/ml.

According to some embodiments of the invention, the effective concentration of the IL6RIL6 chimera in the defined culture medium of some embodiments of the invention is about 100 ng/ml.

It should be noted that the concentration of the IL6RIL6 chimera can vary depending on the purity of the chimeric polypeptide following its synthesis or recombinant expression and those of skills in the art are capable of adjusting the optimal concentration depending on such purity.

According to some embodiments of the invention, the at least one differentiation inhibiting agent is a gp130 agonist.

As used herein the phrase “gp130 agonist” refers to a molecule that binds and activates the gp130 signal transducer and inhibits differentiation of mammalian pluripotent stem cells such as mammalian livestock pluripotent stem cells when cultured in vitro.

According to some embodiments of the invention, the gp130 agonist is selected from the group consisting of leukemia inhibitory factor (LIF), interleukin-6 (IL6), interleukin-11 (IL11), and Ciliary neurotrophic factor (CNTF).

As used herein the term “leukemia inhibitory factor (LIF)” refers to the pleiotropic cytokine which is involved in the induction of hematopoietic differentiation, induction of neuronal cell differentiation, regulator of mesenchymal to epithelial conversion during kidney development, and may also have a role in immune tolerance at the maternal-fetal interface.

The LIF used in the culture medium of some embodiments of the invention can be a purified, synthetic or recombinantly expressed LIF protein [e.g., human LIF polypeptide GenBank Accession No. NP_002300.1 (SEQ ID NO:6); human LIF polynucleotide GenBank Accession No. NM_002309.4 (SEQ ID NO:7). It should be noted that for the preparation of a xeno-free culture medium LIF is preferably purified from a human source or is recombinantly expressed. Recombinant human LIF can be obtained from various sources such as Chemicon, USA (Catalogue No. LIF10100) and AbD Serotec (MorphoSys US Inc, Raleigh, NC 27604, USA). Murine LIF ESGRO® (LIF) can be obtained from Millipore, USA (Catalogue No. ESG1107).

According to some embodiments of the invention, the concentration of LIF in the defined culture medium of some embodiments of the invention is from about 1000 units/ml to about 4,000 units/ml, e.g., from about 2000 units/ml to about 4,000 units/ml, e.g., from about 2000 units/ml to about 3,800 units/ml, e.g., from about 2000 units/ml to about 3,600 units/ml, e.g., from about 2000 units/ml to about 3,500 units/ml, e.g., from about 2000 units/ml to about 3,400 units/ml, e.g., from about 2,500 units/ml to about 3,500 units/ml, e.g., from about 2,800 units/ml to about 3,200 units/ml, e.g., from about 2,900 units/ml to about 3,100 units/ml, e.g., about 3000 units/ml.

According to some embodiments of the invention, the effective concentration of the LIF which is included in the defined culture medium of some embodiments of the invention is about 3000 U/ml.

According to some embodiments of the invention, the concentration of LIF in the culture medium is at least about 1000 units/ml and no more than 5000 units/ml, e.g., at least about 2000 units/ml, e.g., at least about 2100 units/ml, e.g., at least about 2200 units/ml, e.g., at least about 2300 units/ml, e.g., at least about 2400 units/ml, e.g., at least about 2500 units/ml, e.g., at least about 2600 units/ml, e.g., at least about 2700 units/ml, e.g., at least about 2800 units/ml, e.g., at least about 2900 units/ml, e.g., at least about 2950 units/ml and no more than 5000 units/ml, e.g., about 3000 units/ml.

As used herein the term “IL6” (interleukin 6) refers to a cytokine that functions in inflammation and the maturation of B cells.

The IL6 used in the defined culture medium of some embodiments of the invention can be a purified, synthetic or recombinantly expressed IL6 protein such as of the protein set forth by GenBank Accession Nos. NP_000591.1 (SEQ ID NO: 8), NP_001305024.1 (SEQ ID NO: 9) or NP_001358025.1 (SEQ ID NO: 10).

IL6 can be provided from various manufacturers such as Peprotech, R&D Systems.

According to some embodiments of the invention, the effective concentration of IL6 which is included in the defined culture medium of some embodiments of the invention is between 50 ng/ml to 200 ng/ml, e.g., between 70-180 ng/ml, e.g., between 90-150 ng/ml, e.g., between 90-120 ng/ml, e.g., between 90-110 ng/ml.

According to some embodiments of the invention, the effective concentration of IL6 which is included in the defined culture medium of some embodiments of the invention is about 100 ng/ml.

As used herein the term “interleukin 11 (IL11)” refers to a protein member of the gp130 family of cytokines, also known as AGIF and IL-11. Interleukin 11 [e.g., the human IL-11 polypeptide GenBank Accession No. NP_000632.1 (SEQ ID NO:11); human IL-11 polynucleotide GenBank Accession No. NM_000641.2 (SEQ ID NO: 12)] can be obtained from various commercial sources such as R&D Systems or PeproTech.

According to some embodiments of the invention, the effective concentration of IL11 which is included in the defined culture medium of some embodiments of the invention is between 0.2-2 ng/ml, e.g., between 0.5-1.5 ng/ml, e.g., between 0.8-1.2 ng/ml, e.g., between 0.9-1.1 ng/ml.

According to some embodiments of the invention, the effective concentration of IL11 which is included in the defined culture medium of some embodiments of the invention is about 1 ng/ml.

As used herein the term “Ciliay Neurotrophic Factor” (also known as HCNTF; CNTF) refers to a polypeptide hormone whose actions appear to be restricted to the nervous system where it promotes neurotransmitter synthesis and neurite outgrowth in certain neuronal populations. The protein is a potent survival factor for neurons and oligodendrocytes and may be relevant in reducing tissue destruction during inflammatory attacks. CNTF [e.g., the human CNTF polypeptide GenBank Accession No. NP_000605.1 (SEQ ID NO:13); human CNTF polynucleotide GenBank Accession No. NM_000614 (SEQ ID NO:14)] can be obtained from various commercial sources such as R&D Systems or PeproTech.

According to some embodiments of the invention, the effective concentration of CNTF which is included in the defined culture medium of some embodiments of the invention is between 0.2-2 ng/ml, e.g., between 0.5-1.5 ng/ml, e.g., between 0.8-1.2 ng/ml, e.g., between 0.9-1.1 ng/ml.

According to some embodiments of the invention, the effective concentration of CNTF which is included in the defined culture medium of some embodiments of the invention is about 1 ng/ml.

According to some embodiments of the invention, the at least one differentiation inhibiting agent comprises leukemia inhibitory factor (LIF) at a concentration of about 3000 U/ml and basic fibroblast growth factor (bFGF) at a concentration of about 50 ng/ml.

According to some embodiments of the invention, the at least one differentiation inhibiting agent comprises leukemia inhibitory factor (LIF) at a concentration of about 3000 U/ml and basic fibroblast growth factor (bFGF) at a concentration of about 10 ng/ml.

According to some embodiments of the invention, the at least one differentiation inhibiting agent comprises a Wnt3a polypeptide and basic fibroblast growth factor (bFGF).

As used herein the term “WNT3A” refers to a member of the WNT gene family. The WNT gene family consists of structurally related genes which encode secreted signaling proteins. These proteins have been implicated in oncogenesis and in several developmental processes, including regulation of cell fate and patterning during embryogenesis.

The WNT3A mRNA (GenBank Accession NO. NM_033131.3; SEQ ID NO:15) encodes the WNT3A polypeptide (GenBank Accession No. NP_149122.1; SEQ ID NO: 16). The WNT3A polypeptide can be obtained from various manufacturers such as R&D SYSTEMS (e.g., Catalogue No. 5036-WN-010).

According to some embodiments of the invention, the effective concentration of the Wnt3a polypeptide in the defined culture medium of some embodiments of the invention is between 5-20 ng/ml, e.g., between 5-15 ng/ml, e.g., between 6-15 ng/ml, e.g., between 8-13 ng/ml, e.g., between 9-12 ng/ml.

According to some embodiments of the invention, the effective concentration of the Wnt3a polypeptide in the defined culture medium of some embodiments of the invention is about 10 ng/ml.

According to some embodiments of the invention, the effective concentration of the bFGF in the defined culture medium of some embodiments of the invention is in a range of 4-100 ng/ml.

According to some embodiments of the invention, the effective concentration of the bFGF in the defined culture medium of some embodiments of the invention is about 100 ng/ml.

According to some embodiments of the invention, the at least one differentiation inhibiting agent comprises a Wnt3a polypeptide at a concentration of about 10 ng/ml and basic fibroblast growth factor (bFGF) at a concentration in a range of 4-100 ng/ml.

According to some embodiments of the invention, the at least one differentiation inhibiting agent comprises a small molecule.

According to some embodiments of the invention, the small molecule is a protease inhibitor.

As described, the defined culture medium of some embodiments of the invention comprises an effective amount of a protease inhibitor.

As used herein the phrase “effective amount of a protease inhibitor” refers to the amount of protease inhibitor which is sufficient to maintain pluripotent stem cells (e.g., mammalian pluripotent stem cells, e.g., livestock pluripotent stem cells) in a pluripotent state, e.g., for at least 5 passages in culture.

Preferably, the effective amount of the protease inhibitor is sufficient for maintaining the pluripotent stem cells in an undifferentiated state for at least 5 passages in culture.

According to some embodiments of the invention, the protease inhibitor is a reversible protease inhibitor.

According to some embodiments of the invention, the protease inhibitor inhibits serine protease(s).

Non-limiting examples of reversible protease inhibitors which can be used in the defined culture medium of some embodiments of the invention include, but are not limited to phenylmethylsulfonyl fluoride (PMSF), GSK3P inhibitor, Aldehydes-CHO, Arylketones-CO-Aryl, Trifluoromethylketones-COCF3, and Ketocarboxylic acids-COCOOH.

According to some embodiments of the invention, the protease inhibitor is phenylmethylsulfonyl fluoride (PMSF).

PMSF is a serine protease inhibitor commonly used in the preparation of cell lysates. PMSF is rapidly degraded in water and stock solutions are usually made up in anhydrous ethanol, isopropanol, corn oil, or DMSO. Proteolytic inhibition occurs when a concentration between 0.1-1 mM of PMSF is used.

According to some embodiments of the invention, the PMSF used in the defined culture medium of some embodiments of the invention is provided at a concentration of at least about 0.01 mM, e.g., at least about 0.02 mM, e.g., at least about 0.03 mM, e.g., at least about 0.04 mM, e.g., at least about 0.05 mM, e.g., at least about 0.06 mM, e.g., at least about 0.07 mM, e.g., at least about 0.08 mM, e.g., at least about 0.09 mM, e.g., at least about 0.1 mM PMSF.

For example, the PMSF included in the defined culture medium of some embodiments of the invention can be in the range of 0.05 mM to 1 mM, e.g., in the range of 0.05 mM to 0.8 mM, e.g., in the range of 0.05 mM to 0.7 mM, e.g., in the range of 0.05 mM to 0.6 mM, e.g., in the range of 0.05 mM to 0.5 mM, e.g., in the range of 0.06 mM to 0.4 mM, e.g., in the range of 0.07 mM to 0.3 mM, e.g., in the range of 0.07 mM to 0.2 mM, e.g., in the range of 0.07 mM to 0.15 mM, e.g., in the range of 0.07 mM to 0.13 mM, e.g., in the range of 0.08 mM to 0.2 mM, e.g., in the range of 0.09 mM to 0.15 mM, e.g., in the range of 0.09 mM to 0.12 mM, e.g., in the range of 0.09 mM to 0.1 mM, e.g., about 0.1 mM PMSF, e.g., about 0.07 mM PMSF, e.g., about 0.13 mM PMSF.

According to some embodiments of the invention, the protease inhibitor is an irreversible protease inhibitor.

According to some embodiments of the invention, the irreversible protease inhibitor inhibits serine protease(s).

According to some embodiments of the invention, the irreversible protease inhibitor is Tosyl-L-lysyl-chloromethane hydrochloride (TLCK).

TLCK (CAS 4238-41-9) is an irreversible inhibitor of trypsin and trypsin-like serine proteases.

TLCK can be obtained from various suppliers such as abcam (e.g., Catalogue number ab144542), Enzo (Catalogue Number BML-PI121-0200), GENAXXON bioscience Catalogue Number M3375.0100) and the like.

According to some embodiments of the invention, the TLCK is provided in the defined culture medium of some embodiments of the invention at a concentration range from about 0.05 μM to about 1000 μM. For example, between 0.5 μM to about 500 μM, between 0.5 μM to about 400 μM, between 0.5 μM to about 300 μM, between 0.5 μM to about 200 μM, between 0.5 μM to about 100 μM, between 1 μM to about 100 μM, between 5 μM to about 100 μM, between 10 μM to about 100 μM, between 10 μM to about 90 μM, between 10 μM to about 80 μM, between 10 μM to about 70 μM, between 20 μM to about 70 μM, between 30 μM to about 70 μM, between 40 μM to about 70 μM, between 40 μM to about 60 μM, e.g., about 50 μM, about 55 μM or about 60 μM.

According to some embodiments of the invention, the TLCK in the defined culture medium of some embodiments of the invention is provided at a concentration of 20-80 μM.

According to some embodiments of the invention, the TLCK in the defined culture medium of some embodiments of the invention is provided at a concentration of 30-70 μM.

According to some embodiments of the invention, the effective concentration of TLCK in the defined culture medium of some embodiments of the invention is in a range between 40-60 μM.

According to some embodiments of the invention, the TLCK in the defined culture medium of some embodiments of the invention is provided at a concentration of about 50 μM in the defined culture medium of some embodiments of the invention.

According to some embodiments of the invention, the defined culture medium of some embodiments of the invention comprises an effective amount of a protease inhibitor and an effective amount of an IL6RIL6 chimera.

According to some embodiments of the invention, the effective concentration of the IL6RIL6 chimera in a defined medium which further comprises the protease inhibitor is in a range of 50-150 μg/ml.

According to some embodiments of the invention, the effective concentration of the IL6RIL6 chimera in a defined medium which further comprises the protease inhibitor is in a range of 70-130 μg/ml.

According to some embodiments of the invention, the effective concentration of the IL6RIL6 chimera in a defined medium which further comprises the protease inhibitor is in a range of 80-120 μg/ml.

According to some embodiments of the invention, the effective concentration of the IL6RIL6 chimera in a defined medium which further comprises the protease inhibitor is in a range of 50-150 ng/ml.

According to some embodiments of the invention, the at least one differentiation inhibiting agent comprises a gp130 agonist selected from the group consisting of leukemia inhibitory factor (LIF), interleukin-6 (IL6), interleukin-11 (IL11), and Ciliary neurotrophic factor (CNTF) and a protease inhibitor selected from the group consisting of phenylmethylsulfonyl fluoride (PMSF) and Tosyl-L-lysyl-chloromethane hydrochloride (TLCK).

According to some embodiments of the invention, the at least one differentiation inhibiting agent comprises a Wnt3a polypeptide and the IL6RIL6 chimera.

According to some embodiments of the invention, the at least one differentiation inhibiting agent comprises a Wnt3a polypeptide at a concentration in a range of 5-20 ng/ml, and the IL6RIL6 chimera at a concentration in a range of 50-150 μg/ml.

According to some embodiments of the invention, the at least one differentiation inhibiting agent comprises a Wnt3a polypeptide at a concentration in a range of 5-20 ng/ml, and the IL6RIL6 chimera at a concentration in a range of 80-120 μg/ml.

According to some embodiments of the invention, the at least one differentiation inhibiting agent comprises basic fibroblast growth factor (bFGF) and transforming growth factor beta 1 (TGFβ1).

According to some embodiments of the invention, the at least one differentiation inhibiting agent comprises basic fibroblast growth factor (bFGF) and transforming growth factor beta 3 (TGFβ3).

As used herein the phrase “transforming growth factor beta (TGFβ)” refers to any isoform of the transforming growth factor beta (β), which functions through the same receptor signaling system in the control of proliferation, differentiation, and other functions in many cell types. TGFβ acts in inducing transformation and also acts as a negative autocrine growth factor.

There are three known isoforms of TGFβ, TGFβ1 [Human TGFβ1 mRNA sequence GenBank Accession NO. NM_000660.4 (SEQ ID NO:17), polypeptide sequence GenBank Accession No. NP_000651.3 (SEQ ID NO:18)], TGFβ₂ [human TGFβ2 mRNA sequence GenBank Accession NO. NM_001135599.1 isoform 1 (SEQ ID NO:19), or GenBank Accession NO. NM_003238.2 isoform 2 (SEQ ID NO:20); polypeptide sequence GenBank Accession No. NP_001129071.1 isoform 2 (SEQ ID NO:21) or GenBank Accession NO. NP_003229.1 isoform 2 (SEQ ID NO:22] or TGFβ₃ [human TGFβ3 mRNA sequence GenBank Accession NO. NM_003239.2 (SEQ ID NO:23), polypeptide sequence GenBank Accession No. NP_003230.1 (SEQ ID NO:24)]. The TGFβ isoforms can be obtained from various commercial sources such as R&D Systems Minneapolis MN, USA, and Sigma, St Louis, MO, USA.

According to some embodiments of the invention, the effective concentration of the TGFβ1 in the defined culture medium of some embodiments of the invention is in a range of 0.06-0.24 ng/ml, e.g., in a range of 0.08-0.20 ng/ml, e.g., in a range of 0.1-0.15 ng/ml, e.g., in a range of 0.11-0.13 ng/ml TGFβ1.

According to some embodiments of the invention, the effective concentration of the TGFβ1 in the defined culture medium of some embodiments of the invention is about 0.12 ng/ml.

According to some embodiments of the invention, in the defined culture medium which comprises the TGFβ1 and bFGF, the effective concentration of the bFGF is in a range of 4-20 ng/ml, e.g., about 10 ng/ml bFGF.

According to some embodiments of the invention, in the defined culture medium which comprises the TGFβ1 and bFGF, the effective concentration of the bFGF is in a range of 70-130 ng/ml, e.g., about 100 ng/ml bFGF.

According to some embodiments of the invention, the effective concentration of the TGFβ3 in the defined culture medium of some embodiments of the invention is in a range of 0.5-4 ng/ml, e.g., in a range of 4 ng/ml, e.g., in a range of 1.5-3.5 ng/ml, e.g., in a range of 1.5-2.5 ng/ml TGFβ3.

According to some embodiments of the invention, the effective concentration of the TGFβ3 in the defined culture medium of some embodiments of the invention is about 2 ng/ml.

According to some embodiments of the invention, the defined culture medium further comprises ascorbic acid.

According to some embodiments of the invention the concentration of ascorbic acid in the defined medium is in a range of 8-600 microgram/milliliter (μg/ml), e.g., in the range of 10-15 μg/ml, e.g., in the range of 40-60 μg/ml, e.g., in the range of 450-550 μg/ml, e.g., about 500 μg/ml.

According to some embodiments of the invention the defined culture medium comprises the IL6RIL6 chimera at a concentration in a range of 50-150 pg/ml and ascorbic acid at a concentration of 450-550 μg/ml.

According to some embodiments of the invention the defined culture medium comprises a basal medium (e.g., 95% DMEM/F12 (or KO-DMEM)) supplemented with insulin (at a concentration range of 0.34×10⁻³ mM to 1.88×10⁻³ mM), transferrin (at a concentration range of 0.137×10⁻⁴ mM to 0.66×10⁻⁴ mM, a lipid mixture at concentration of 0.5% [volume/volume (v/v)] to 1.2% v/v, bovine serum albumin (BSA) at a concentration range of BSA 0.4% (v/v) to 0.7% (v/v), and ascorbic acid 450-550 μg/ml, wherein the serum replacement is devoid of selenium.

According to some embodiments of the invention the defined culture medium comprises a basal medium (e.g., 95% DMEM/F12 (or KO-DMEM)) supplemented with insulin (at a concentration range of 0.34×10⁻³ mM to 1.88×10⁻³ mM), transferrin (at a concentration range of 0.137×10⁻⁴ mM to 0.66×10⁻⁴ mM, a lipid mixture at concentration of 0.5% [volume/volume (v/v)] to 1.2% v/v, bovine serum albumin (BSA) at a concentration range of BSA 0.4% (v/v) to 0.7% (v/v), ascorbic acid 450-550 μg/ml, and the IL6RIL6 chimera at a concentration in a range of 50-150 μg/ml, wherein the serum replacement is devoid of selenium.

According to some embodiments of the invention the defined culture medium comprises a basal medium (e.g., 95% DMEM/F12 (or KO-DMEM)) supplemented with ITS [Insulin (at a concentration range of 0.34×10⁻³ mM to 1.88×10⁻³ mM), transferrin (at a concentration range of 0.137×10⁻⁴ mM to 0.66×10⁻⁴ mM), and Selenium (at a concentration range of 2.11×10⁻⁵ mM to 5.9×10⁻⁵ mM)], a lipid mix at a concentration of 0.5-1.2% v/v, ascorbic acid in the range of 450-550 μg/ml, and bovine serum albumin (at a concentration of 0.4% to 0.7%.

According to some embodiments of the invention the defined culture medium comprises a basal medium (e.g., 95% DMEM/F12 (or KO-DMEM)) supplemented with ITS [Insulin (at a concentration range of 0.34×10⁻³ mM to 1.88×10⁻³ mM), transferrin (at a concentration range of 0.137×10⁻⁴ mM to 0.66×10⁻⁴ mM), and Selenium (at a concentration range of 2.11×10⁻⁵ mM to 5.9×10⁻⁵ mM)], a lipid mix at a concentration of 0.5-1.2% v/v, ascorbic acid in the range of 450-550 μg/ml, the IL6RIL6 chimera at a concentration in a range of 50-150 μg/ml, and bovine serum albumin (at a concentration of 0.4% to 0.7%.

According to some embodiments of the invention the defined culture medium of some embodiments of the invention comprises 95% DMEM/F12 (or KO-DMEM) and supplemented with ITS [Insulin (at a concentration range of 0.34×10⁻³ mM to 1.88×10⁻³ mM), transferrin (at a concentration range of 0.137×10⁻⁴ mM to 0.66×10⁻⁴ mM), and Selenium (at a concentration range of 2.11×10⁻⁵ mM to 5.9×10⁻⁵ mM)]; fatty acid mix [including Linoleic Acid at a concentration in a range of 0.47-0.63×10⁻⁴ mM, Lipoic Acid at a concentration in a range of 1-1.33×10⁻⁴ mM, Arachidonic Acid at a concentration in a range of 0.32-0.43×10⁻⁵ mM, Cholesterol at a concentration in a range of 0.28-0.37×10⁻³ mM, DL-alpha tocopherol-acetate at a concentration in a range of 0.72-0.96×10⁻³ mM, Linolenic Acid at a concentration in a range of 1.74-2.33×10⁻⁵ mM Myristic Acid at a concentration in a range of 2.14-2.86×10⁻⁵ mM, Oleic Acid at a concentration in a range of 1.73-2.31×10⁻⁵ mM, Palmitic Acid at a concentration in a range of 1.91-2.55×10⁻⁵ mM, Palmitoleic acid at a concentration in a range of 1.92-2.571×10⁻⁵ mM, and Stearic Acid at a concentration in a range of 1.72-2.29×10⁻⁵ mM]; ascorbic acid in the range of 450-550 μg/ml; and bovine serum albumin (at a concentration of 0.4% to 0.7% v/v).

According to some embodiments of the invention the defined culture medium of some embodiments of the invention comprises 95% DMEM/F12 (or KO-DMEM) and supplemented with ITS [Insulin (at a concentration range of 0.34×10⁻³ mM to 1.88×10⁻³ mM), transferrin (at a concentration range of 0.137×10⁻⁴ mM to 0.66×10⁻⁴ mM), and Selenium (at a concentration range of 2.11×10⁻⁵ mM to 5.9×10⁻⁵ mM)], a fatty acid mix [including Linoleic Acid at a concentration in a range of 0.47-0.63×10⁻⁴ mM, Lipoic Acid at a concentration in a range of 1-1.33×10⁻⁴ mM, Arachidonic Acid at a concentration in a range of 0.32-0.43×10⁻⁵ mM, Cholesterol at a concentration in a range of 0.28-0.37×10⁻³ mM, DL-alpha tocopherol-acetate at a concentration in a range of 0.72-0.96×10⁻³ mM, Linolenic Acid at a concentration in a range of 1.74-2.33×10⁻⁵ mM Myristic Acid at a concentration in a range of 2.14-2.86×10⁻⁵ mM, Oleic Acid at a concentration in a range of 1.73-2.31×10⁻⁵ mM, Palmitic Acid at a concentration in a range of 1.91-2.55×10⁻⁵ mM, Palmitoleic acid at a concentration in a range of 1.92-2.571×10⁻⁵ mM, and Stearic Acid at a concentration in a range of 1.72-2.29×10⁻⁵ mM], ascorbic acid in the range of 450-550 μg/ml, the IL6RIL6 chimera at a concentration in a range of 50-150 pg/ml, and bovine serum albumin (at a concentration of 0.4% to 0.7% v/v).

According to some embodiments of the invention the at least one differentiation inhibiting agent comprises the IL6RIL6 chimera at a concentration in a range of 50-150 μg/ml, ascorbic acid at a concentration of 450-550 μg/ml and bFGF at a concentration of 30-70 ng/ml.

According to some embodiments of the invention the defined culture medium comprises a basal medium (e.g., 95% DMEM/F12 (or KO-DMEM)) supplemented with insulin (at a concentration range of 0.34×10⁻³ mM to 1.88×10⁻³ mM), transferrin (at a concentration range of 0.137×10⁻⁴ mM to 0.66×10⁻⁴ mM, a lipid mixture at concentration of 0.5% [volume/volume (v/v)] to 1.2% v/v, bovine serum albumin (BSA) at a concentration range of BSA 0.4% (v/v) to 0.7% (v/v), ascorbic acid 450-550 μg/ml, the IL6RIL6 chimera at a concentration in a range of 50-150 pg/ml, and bFGF at a concentration of 30-70 ng/ml, wherein the serum replacement is devoid of selenium.

According to some embodiments of the invention the defined culture medium comprises a basal medium (e.g., 95% DMEM/F12 (or KO-DMEM)) supplemented with ITS [Insulin (at a concentration range of 0.34×10⁻³ mM to 1.88×10⁻³ mM), transferrin (at a concentration range of 0.137×10⁻⁴ mM to 0.66×10⁻⁴ mM), and Selenium (at a concentration range of 2.11×10⁻⁵ mM to 5.9×10⁻⁵ mM)], a lipid mix at a concentration of 0.5-1.2% v/v, ascorbic acid in the range of 450-550 μg/ml, the IL6RIL6 chimera at a concentration in a range of 50-150 μg/ml, bFGF at a concentration of 30-70 ng/ml, and bovine serum albumin (at a concentration of 0.4% to 0.7%.

According to some embodiments of the invention, the defined culture medium of the invention is not suitable for cryopreservation of cells.

As used herein the term “cryopreservation” refers to preservation of cells under freezing conditions such as at a temperature which is below the water freezing point of 0° C., e.g., lower than −5° C., −10° C., −18° C., −20° C., −50° C. or −70° C. (the “−” sign represents a negative value).

According to some embodiments of the invention, the defined culture medium of the invention is devoid of a cryoprotectant.

A cryoprotectant is a substance used to protect biological tissue or cells from a freezing damage which can result from formation of ice.

Known conventional cryoprotectants include, but are not limited to glycols such as ethylene glycol, propylene glycol and glycerol. Dimethyl sulfoxide (DMSO) is also regarded as a conventional cryoprotectant. Glycerol and DMSO have been used to reduce ice formation in sperm, oocytes, and embryos that are cold-preserved in liquid nitrogen. Trehalose is non-reducing sugar produced by yeasts and insects and is used as a cryoprotectant.

According to some embodiments of the invention, the defined culture medium of the invention is devoid of a cryoprotectant such as Dimethyl sulfoxide (DMSO), sucrose, galactose, Trehalose, a glycol (e.g., ethylene glycol, propylene glycol, methanol and glycerol).

As described, the defined culture medium of some embodiments of the invention is capable of maintaining mammalian livestock pluripotent stem cells in an undifferentiated state for at least 5 passages in culture.

As described, the defined culture medium of some embodiments of the invention is capable of maintaining human pluripotent stem cells in an undifferentiated state for at least 5 passages in culture.

According to an aspect of some embodiments of the invention there is provided a cell culture comprising the defined culture medium of some embodiments of the invention and cells.

According to some embodiments of the invention the cells are stem cells.

According to some embodiments of the invention the cells are mammalian pluripotent stem cells.

According to some embodiments of the invention, the pluripotent stem cell (PSC) is a mammalian pluripotent stem cell, e.g., a human pluripotent stem cell.

According to some embodiments of the invention the cells are mammalian livestock pluripotent stem cells.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells).

The phrase “pluripotent stem cells” refers to cells which can differentiate into all three embryonic germ layers, i.e., ectoderm, endoderm and mesoderm, or remaining in an undifferentiated state.

The phrase “pluripotent stem cells” may read on embryonic stem cells (ESCs) and/or induced pluripotent stem cells (iPS cells).

The phrase “embryonic stem cells” as used herein refers to cells which are obtained from the embryonic tissue formed after gestation (e.g., blastocyst) before implantation (i.e., a pre-implantation blastocyst); extended blastocyst cells (EBCs) which are obtained from a post-implantation/pre-gastrulation stage blastocyst and/or embryonic germ (EG) cells which are obtained from the genital tissue of a fetus any time during gestation, preferably before 10 weeks of gestation.

According to some embodiments of the invention, the pluripotent stem cells of the invention are embryonic stem cells, such as from a mammalian origin, such as mammalian livestock pluripotent stem cells or human pluripotent stem cells.

The embryonic stem cells of the invention can be obtained using well-known cell-culture methods. For example, a mammalian embryonic stem cells can be isolated from a mammalian blastocyst. Mammalian blastocysts can be obtained from in vivo preimplantation embryos or from in vitro fertilized (IVF) embryos. Alternatively, a single cell mammalian embryo can be expanded to the blastocyst stage. For the isolation of mammalian ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 6 to 15 days the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. Methods of preparation human ES cells are described in Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998]. Methods of preparation mammalian livestock ES cells are described in Toshihiko Ezashi et al., 2016 (Annu. Rev. Anim. Biosci. 4: 223-253 and in references cited therein, which are fully incorporated herein by reference).

It will be appreciated that commercially available stem cells can also be used with this aspect of the present invention. Human ES cells can be purchased from the NIH human embryonic stem cells registry (www(dot)escr(dot)nih(dot)gov). Non-limiting examples of commercially available embryonic stem cell lines are BGO1, BG02, BG03, BG04, CY12, CY30, CY92, CY10, TE03, TE04 and TE06.

Human extended blastocyst cells (EBCs) can be obtained from a human blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Mammalian livestock extended blastocyst cells (EBCs) can be obtained from a mammalian livestock blastocyst of at least 7 days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least 4 and no more than 21 days post fertilization (i.e., prior to the gastrulation event) in vitro using standard pluripotent stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

Embryonic germ (EG) cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

The phrase “induced pluripotent stem (iPS) cell” (or embryonic-like stem cell) as used herein refers to a proliferative and pluripotent stem cell which is obtained by de-differentiation of a somatic cell (e.g., an adult somatic cell).

According to some embodiments of the invention, the iPS cell is characterized by a proliferative capacity which is similar to that of ESCs and thus can be maintained and expanded in culture for an almost unlimited time.

IPS cells can be endowed with pluripotency by genetic manipulation which re-program the cell to acquire embryonic stem cells characteristics. For example, the iPS cells of the invention can be generated from somatic cells such as fibroblasts, hepatocytes, gastric epithelial cells by induction of expression of Oct-4, Sox2, Kfl4 and c-Myc in a somatic cell essentially as described in Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131: 861-872, each of which is fully incorporated by reference in its entirety. Additionally or alternatively, the iPS cells of the invention can be generated from somatic cells by induction of expression of OCT4, Sox2, Nanog and Lin28 essentially as described in Yu Junying et al. (Science 318:1917-1920, 2007), and Nakagawa et al, 2008 (Nat Biotechnol. 26(1):101-106). It should be noted that the genetic manipulation (re-programming) of the somatic cells can be performed using any known method such as using plasmids or viral vectors, or by derivation without any integration to the genome [Yu J, et al., Science. 2009, 324: 797-801]. Other embryonic-like stem cells can be generated by nuclear transfer to oocytes, fusion with embryonic stem cells or nuclear transfer into zygotes if the recipient cells are arrested in mitosis. WO 03/046141 A2 (Advanced Cell Tech Inc. 5 Jun. 2003) teaches generation of activated human embryos by parthenogenesis as well as by somatic cell nuclear transfer.

The iPS cells of the invention can be obtained by inducing de-differentiation of embryonic fibroblasts [Takahashi and Yamanaka, 2006 Cell. 2006, 126(4):663-676; Meissner et al, 2007 Nat Biotechnol. 2007, 25(10):1177-1181], fibroblasts formed from hESCs [Park et al, 2008 Nature. 2008, 451(7175):141-146], Fetal fibroblasts [Yu et al, 2007 Science. 2009, 324(5928):797-801; Park et al, 2008 (supra)], foreskin fibroblast [Yu et al, 2007 (supra); Park et al, 2008 (supra)], adult dermal and skin tissues [Hanna et al, 2007 Science. 2007, 318(5858):1920-1923; Lowry et al, 2008 Proc Natl Acad Sci USA, 105(8):2883-2888], b-lymphocytes [Hanna et al 2007 (supra)] and adult liver and stomach cells [Aoi et al, 2008 Science. 2008 Aug. 1; 321(5889):699-702].

IPS cell lines are also available via cell banks such as the WiCell bank. Non-limiting examples of commercially available iPS cell lines include the iPS foreskin clone 1 [WiCell Catalogue No. iPS(foreskin)-1-DL-1], the iPSIMR90 clone 1 [WiCell Catalogue No. iPS(IMR90)-1-DL-1], and the iPSIMR90 clone 4 [WiCell Catalogue No. iPS(IMR90)-4-DL-1].

The defined culture medium of some embodiments of the invention can be used to derive a pluripotent stem cell line.

As used herein the phrase “deriving” with respect to “a mammalian pluripotent stem cells line” refers to generating a population of mammalian pluripotent stem cells from at least one stem cell (e.g., a blastomere (a cell of a blastocyst), an epiblast cell or a late-stage pluripotent stem cell) that is isolated from a single mammalian embryo (e.g., from an ex-vivo cultured mammalian embryo such as an ex-vivo cultured bovine embryo).

As used herein the phrase “epiblast cells” refers to cells of the embryonic epiblast. These cells are pluripotent and therefore capable of differentiating into all three embryonic germ layers.

As used herein the phrase “late stage pluripotent stem cells” refers to cells which are derived from the late epiblast stage until gastrulation. These cells are pluripotent and therefore capable of differentiating into all three embryonic germ layers.

According to some embodiments of the invention, the epiblast cell and/or the late-stage pluripotent stem cell are characterized by a large nucleus to cytoplasm ratio.

As used herein the phrase “mammalian livestock” refers to a domesticated mammalian animal which is typically used as a source of food, such as meat and/or milk.

According to some embodiments of the invention, the mammalian livestock is a ruminant mammalian livestock.

According to some embodiments of the invention, the mammalian livestock is a non-ruminant mammalian livestock.

According to some embodiments of the invention, the ruminant mammalian livestock is selected from the group consisting of a Bovinae subfamily, sheep, goat, deer, and camel.

According to some embodiments of the invention, the ruminant mammalian livestock of the Bovinae subfamily is cattle or a yak.

According to some embodiments of the invention, the ruminant mammalian livestock of the Bovinae subfamily is cattle.

According to some embodiments of the invention, the cattle is buffalo, bison or cow (bovine).

According to some embodiments of the invention, the mammalian livestock is cow (bovine).

According to some embodiments of the invention, the cattle is cow (bovine).

According to some embodiments of the invention, the non-ruminant mammalian livestock is selected from the group pig, rabbit, and horse.

According to some embodiments of the invention, the mammalian livestock pluripotent stem cell is derived from a delayed bovine blastocyst, e.g., by ex-vivo culturing a mammalian livestock embryo of at least 7 days post-fertilization for a culturing period of at least 4 days and no more than until 21 days post-fertilization so at to obtain an embryo comprising an epiblast cell and/or a late stage pluripotent stem cell.

Non-limiting examples of mammalian livestock pluripotent stem cell lines which are derived from a delayed bovine blastocyst include the BVN1, BVN2, BVN5 and BVN6.

According to some embodiments of the invention, the mammalian livestock pluripotent stem cell is derived from a bovine blastocyst such as by culturing a mammalian livestock embryo of 7 days post fertilization directly on a feeder cell layer (such as MEF feeder layer) or a feeder-free matrix (such as a Matrigel matrix, fibronectin matrix, laminin matrix, collagen matrix, Elastin matrix, and Vitronectin matrix).

Non-limiting examples of mammalian livestock pluripotent stem cells which are derived from a bovine blastocyst include, but are not limited to bovine embryonic stem cells (ESCs) such as BVN3 and BVN4.

Derivation of Bovine embryonic stem cells can be performed essentially as described in Bogliotti Y S, et al. 2018 (Efficient derivation of stable primed pluripotent embryonic stem cells from bovine blastocysts. PNAS, 115 (9): 2090-2095), which uses whole embryo culturing at day 7 of bovine embryo.

According to an aspect of some embodiments of the invention there is provided a mammalian livestock pluripotent stem cell line which is derived from a mammalian livestock blastocyst (e.g., from a blastomere), an epiblast cell or a late-stage pluripotent stem cell by ex-vivo culturing the cell in the defined culture medium of some embodiments of the invention.

According to some embodiments of the invention the mammalian livestock pluripotent stem cell line is the BVN3 cell line which was derived by the present inventor using the defined culture medium of some embodiments of the invention which comprises the IL6RIL6R chimera.

According to some embodiments of the invention, a mammalian livestock pluripotent stem cell line can be obtained from a 7-day bovine embryo at the blastocyst stage. After removal of the zona pellucida (ZP) the 7-day bovine embryo is plated on feeder cells (e.g., MEFs) in the presence of a defined culture medium according to some embodiments of the invention, e.g., a culture medium which comprises the IL6RIL6R chimera and 5% serum replacement. At day 17 of the embryo the cultured cells with PSCs morphology can be collected mechanically under a microscope and re-plated on a fresh feeder cells layer (e.g., MEFs).

According to some embodiments of the invention, the mammalian livestock pluripotent stem cell is an induced pluripotent stem cell (iPSC) derived from a mammalian livestock somatic cell which was subject to de-differentiation. De-differentiation can be conducted using a commercial reprograming kit [such as Epi5 episomal iPSCs kit (Thrmo-Fisher), Simplicon RNA reprograming Kit (Merck-Millipore), Stemcca kit (Merck-Millipore), Stemgent stemRNA 3ed reprograming kit (Reprocelll) or CytoTune™ kit (Life Technology)], each of which according to manufacturer's instructions.

Non-limiting examples of mammalian livestock iPSC include cell lines derived from bovine fetal tissue and from endometrium epithelial cells such as iBVN1.4, iBVN1.14 and iBVN1.15.

According to some embodiments of the invention, the mammalian livestock iPSCs are generated by transient expression of the reprogramming factors (e.g., the Oct3/4, Sox2, cMyc, and Klf4 genes).

According to some embodiments of the invention, once generated, the mammalian livestock iPSCs are not dependent on a persistent expression of the reprogramming factors (e.g., Oct3/4, Sox2, cMyc, and Klf4 genes) in order to maintain their pluripotency and undifferentiated state.

According to some embodiments of the invention, once generated, the vector that encodes the reprogramming factors is shut down and there is no continued expression of the reprogramming factors (e.g., the Oct3/4, Sox2, cMyc, and Klf4 genes).

According to an aspect of some embodiments of the invention there is provided a method of maintaining mammalian livestock pluripotent stem cells in an undifferentiated state, comprising culturing the mammalian livestock pluripotent stem cells in the defined culture medium of some embodiments of the invention.

According to some embodiments of the invention, the method further comprising passaging the mammalian livestock pluripotent stem cells for at least one time.

As used herein the term “passage” or “passaging” as used herein refers to splitting the cells in the culture vessel to 2 or more culture vessels, typically including addition of fresh culture medium. Passaging is typically done when the cells reach a certain density in culture.

According to some embodiments of the invention, passaging is effected every 5-21 days during the culturing.

According to some embodiments of the invention, passaging comprises splitting the mammalian livestock pluripotent stem cells in a 1 to 2, or a 2 to 3 ratio before further culturing the cells.

According to some embodiments of the invention, passaging is performed by mechanical passaging.

As used herein the phrase “mechanical dissociation” refers to separating the pluripotent stem cell clumps to single cells by employing a physical force rather than an enzymatic activity.

For mechanical dissociation, a pellet of pluripotent stem cells (which may be achieved by centrifugation of the cells) or an isolated pluripotent stem cells clump can be dissociated by pipetting the cells up and down in a small amount of medium (e.g., 0.2-1 ml). For example, pipetting can be performed for several times (e.g., between 3-20 times) using a tip of a 200 μl or 1000 μl pipette.

Additionally or alternatively, mechanical dissociation of large pluripotent stem cells clumps can be performed using a device designed to break the clumps to a predetermined size. Such a device can be obtained from CellArtis Goteborg, Sweden. Additionally or alternatively, mechanical dissociation can be manually performed using a needle such as a 27 g needle (BD Microlance, Drogheda, Ireland) while viewing the clumps under an inverted microscope.

According to some embodiments of the invention, passaging is effected under conditions devoid of enzymatic dissociation.

According to some embodiments of the invention, the method further comprising mechanically passaging the pluripotent stem cells for at least 2 passages, e.g., at least 3 passages, e.g., at least 4 passages, e.g., at least 5 passages to thereby obtain an expanded population of pluripotent stem cells.

According to some embodiments of the invention, passaging is performed by enzymatic dissociation of cell clumps.

Enzymatic digestion of pluripotent stem cells clump(s) can be performed by subjecting the clump(s) or the colonies to an enzyme such as type IV Collagenase (Worthington biochemical corporation, Lakewood, NJ, USA) and/or Dispase (Invitrogen Corporation products, Grand Island NY, USA). The time of incubation with the enzyme depends on the size of cell clumps or the colonies present in the cell culture. Typically, when pluripotent stem cells cell clumps are dissociated every 5-21 days while in culture, incubation of 20-60 minutes with 1.5 mg/ml type IV Collagenase results in small cell clumps which can be further cultured in the undifferentiated state. Alternatively, pluripotent stem cells clumps can be subjected to incubation of about 25 minutes with 1.5 mg/ml type IV Collagenase followed by five minutes incubation with 1 mg/ml Dispase.

According to some embodiments of the invention, the method further comprises enzymatic passaging the population of pluripotent stem cells for at least 2 passages, e.g., at least 3 passages, e.g., at least 4 passages, e.g., at least 5 passages to thereby obtain an expanded population of pluripotent stem cells.

According to some embodiments of the invention, the population of pluripotent stem cells is expanded in an undifferentiated state for an extended time period while being serially passaged.

According to some embodiments of the invention, the extended time period is at least one two weeks, e.g., at least one month, e.g., at least 3, 4, 5, 6, 7 months or more while in culture.

According to some embodiments of the invention, the serial passaging of the pluripotent stem cells is performed every 5-21 days, e.g., every 5-15 days, e.g., every 5-10 days, e.g., every 5-7 days.

According to some embodiments of the invention, passaging the pluripotent stem cells is performed by enzymatic passaging (e.g., using type IV collagenase, Dispase, TryPLE trypsin).

According to some embodiments of the invention, the culturing is performed on feeder cell layers.

According to some embodiments of the invention, culturing the mammalian livestock pluripotent stem cell is performed on a two-dimensional culture system.

According to some embodiments of the invention, the two-dimensional culture system comprises a feeder-free matrix.

According to some embodiments of the invention, culturing is performed on an extracellular matrix.

According to some embodiments of the invention, the culturing is performed in a suspension culture devoid of substrate adherence.

As used herein the phrase “suspension culture” refers to a culture in which the pluripotent stem cells are suspended in a medium rather than adhering to a surface.

Thus, the culture of the present invention is “devoid of substrate adherence” in which the pluripotent stem cells are capable of expanding without adherence to an external substrate such as components of extracellular matrix, a glass microcarrier or beads.

It should be noted that some protocols of culturing pluripotent stem cells such as ESCs and iPS cells include microencapsulation of the cells inside a semipermeable hydrogel membrane, which allows the exchange of nutrients, gases, and metabolic products with the bulk medium surrounding the capsule (for details see e.g., U.S. Patent Application No. 20090029462 to Beardsley et al.).

According to some embodiments of the invention, the pluripotent stem cells cultured in the suspension culture are devoid of cell encapsulation.

According to some embodiments of the invention, the culture medium and/or the conditions for culturing the pluripotent stem cells in suspension are devoid of a protein carrier.

According to some embodiments of the invention the suspension culture is devoid of substrate adherence and devoid of protein carrier.

As used herein the phrase “protein carrier” refers to a protein which acts in the transfer of proteins or nutrients (e.g., minerals such as zinc) to the cells in the culture. Such protein carriers can be, for example, albumin (e.g., bovine serum albumin), Albumax (lipid enriched albumin) or plasmanate (human plasma isolated proteins).

Culturing in suspension is effected by plating the pluripotent stem cells in a culture vessel at a cell density which promotes cell survival and proliferation but limits differentiation.

Typically, a plating density of between about 5×10⁴-2×10⁵ cells per ml is used. It will be appreciated that although single-cell suspensions of stem cells are usually seeded, small clusters such as 10-200 cells may also be used.

In order to provide the PSCs with sufficient and constant supply of nutrients and growth factors while in the suspension culture, the culture medium can be replaced on a daily basis, or, at a pre-determined schedule such as every 2-3 days. For example, replacement of the culture medium can be performed by subjecting the PSCs suspension culture to centrifugation for about 3 minutes at 80 g, and resuspension of the formed PSCs pellet in a fresh medium. Additionally, or alternatively, a culture system in which the culture medium is subject to constant filtration or dialysis so as to provide a constant supply of nutrients or growth factors to the PSCs may be employed.

Since large clusters of PSCs may cause cell differentiation, measures are taken to avoid large PSCs aggregates. Preferably, the formed PSC clumps are dissociated every 5-7 days and the single cells or small clumps of cells are either split into additional culture vessels (i.e., passaged) or remained in the same culture vessel yet with additional culture medium. For dissociation of large PSCs clumps, a pellet of PSCs (which may be achieved by centrifugation as described hereinabove) or an isolated PSCs clump can be subject to enzymatic digestion and/or mechanical dissociation as explained above.

As described, the mammalian pluripotent stem cells are capable of differentiation into the endoderm, mesoderm and ectoderm embryonic germ layers.

Differentiation of the pluripotent stem cells of some embodiments of the invention into the endoderm, mesoderm and ectoderm embryonic germ layers can be performed by direct differentiation in cell culture, by differentiation into embryoid bodies and/or by teratoma formations.

The mammalian pluripotent stem cells (e.g., from livestock) which are included by the cell cultures of some embodiments of the invention, and/or which are used by the methods of some embodiments of the invention can be can be used as a source for generating differentiated, lineage-specific cells. Such cells can be obtained directly from the pluripotent stem cells by subjecting the PSCs to various differentiation signals (e.g., cytokines, hormones, growth factors) or indirectly, via the formation of embryoid bodies and the subsequent differentiation of cells of the EBs to lineage-specific cells.

Thus, according to an aspect of the some embodiments of the invention there is provided a method of generating embryoid bodies from pluripotent stem cells. The method is effected by (a) culturing the pluripotent stem cells of some embodiments of the invention according to the method of some embodiment of the invention to thereby obtain expanded, undifferentiated pluripotent stem cells; and (b) subjecting the expanded, undifferentiated pluripotent stem cells to culturing conditions suitable for differentiating the stem cells to embryoid bodies, thereby generating the embryoid bodies from the pluripotent stem cells.

As used herein the phrase “embryoid bodies” refers to morphological structures comprised of a population of ESCs, extended blastocyst cells (EBCs), embryonic germ cells (EGCs) and/or induced pluripotent stem cells which have undergone differentiation. EBs formation initiates following the removal of differentiation blocking factors from the pluripotent stem cell cultures. In the first step of EBs formation, the pluripotent stem cells proliferate into small masses of cells which then proceed with differentiation. In the first phase of differentiation, following a predetermined period in culture (e.g., 1-4 days in culture for either human ESCs or human iPS cells; e.g., 1-4 days in culture of mammalian livestock pluripotent stem cells), a layer of endodermal cells is formed on the outer layer of the small mass, resulting in “simple EBs”. In the second phase, following 3-20 days post-differentiation, “complex EBs” are formed. Complex EBs are characterized by extensive differentiation of ectodermal and mesodermal cells and derivative tissues.

During the culturing period, EBs are further monitored for their differentiation state. Cell differentiation can be determined upon examination of cell or tissue-specific markers which are known to be indicative of differentiation. For example, EB-derived-differentiated cells may express the neurofilament 68 KD which is a characteristic marker of the ectoderm cell lineage.

The differentiation level of the EB cells can be monitored by following the loss of expression of OCT-4, and the increased expression level of other markers such as α-fetoprotein, NF-68 kDa, α-cardiac and albumin. Methods useful for monitoring the expression level of specific genes are well known in the art and include RT-PCR, semi-quantitative RT-PCR, Northern blot, RNA in situ hybridization, Western blot analysis and immunohistochemistry.

Thus, the method according to some embodiments of the invention involves the culturing of the pluripotent stem cells of some embodiments of the invention in any of the culture media described hereinabove in order to obtain expanded, undifferentiated pluripotent stem cells and then subjecting the expanded, undifferentiated pluripotent stem cells to culturing conditions suitable for differentiating the pluripotent stem cells to embryoid bodies. Such differentiation-promoting culturing conditions are substantially devoid of differentiation inhibitory factors which are employed when pluripotent stem cells are to be expanded in an undifferentiated state, such as TGFβ1, TGFβ₃, ascorbic acid, gp130 agonists, e.g., IL-11, CNTF, oncostatin, bFGF and/or the IL6RIL6 chimera.

For EBs formation, the pluripotent stem cells (ESCs or iPS cells) are removed from their feeder-free-culturing systems or suspension cultures and are transferred to a suspension culture in the presence of a culture medium containing serum or serum replacement and being devoid of differentiation-inhibitory factors. For example, a culture medium suitable for EBs formation may include a basic culture medium (e.g., Ko-DMEM or DMEM/F12) supplemented with 20% FBSd (HyClone, Utah, USA), 1 mM L-glutamine, 0.1 mM β-mercaptoethanol, and 1% non-essential amino acid stock.

Monitoring the formation of EBs is within the capabilities of those skilled in the art and can be effected by morphological evaluations (e.g., histological staining) and determination of expression of differentiation-specific markers [e.g., using immunological techniques or RNA-based analysis (e.g., RT-PCR, cDNA microarray)].

It will be appreciated that in order to obtain lineage-specific cells from the EBs, cells of the EBs can be further subjected to culturing conditions suitable for lineage-specific cells.

According to some embodiments of the invention, for generating lineage-specific cells from the pluripotent stem cells, the method further includes step (c) of subjecting cells of the embryoid bodies to culturing conditions suitable for differentiating and/or expanding lineage specific cells; thereby generating the lineage-specific cells from the embryonic stem cells.

As used herein the phrase “culturing conditions suitable for differentiating and/or expanding lineage specific cells” refers to a combination of culture system, e.g., feeder-free matrix or a suspension culture and a culture medium which are suitable for the differentiation and/or expansion of specific cell lineages derived from cells of the EBs. Non-limiting examples of such culturing conditions are further described hereinunder.

It will be appreciated that since EBs are complex structures, differentiation of EBs into specific differentiated cells, tissue or organ may require isolation of lineage specific cells from the EBs.

According to some embodiments of the invention, the method of this aspect of the invention further includes isolating lineage specific cells following step (b).

As used herein, the phrase “isolating lineage specific cells” refers to the enrichment of a mixed population of cells in a culture with cells predominantly displaying at least one characteristic associated with a specific lineage phenotype. It will be appreciated that all cell lineages are derived from the three embryonic germ layers. Thus, for example, hepatocytes and pancreatic cells are derived from the embryonic endoderm, osseous, cartilaginous, elastic, fibrous connective tissues, myocytes, myocardial cells, bone marrow cells, vascular cells (namely endothelial and smooth muscle cells), and hematopoietic cells are differentiated from embryonic mesoderm and neural, retina and epidermal cells are derived from the embryonic ectoderm.

Such isolation may be effected by sorting of cells of the EBs via fluorescence activated cell sorter (FACS) or mechanical separation of cells, tissues and/or tissue-like structures contained within the EBs.

According to some preferred embodiments of the invention, isolating lineage specific cells is effected by sorting of cells of the EBs via fluorescence activated cell sorter (FACS).

Methods of isolating EB-derived-differentiated cells via FACS analysis are known in the art. According to one method, EBs are disaggregated using a solution of Trypsin and EDTA (0.025% and 0.01%, respectively), washed with 5% fetal bovine serum (FBS) in phosphate buffered saline (PBS) and incubated for 30 min on ice with fluorescently-labeled antibodies directed against cell surface antigens characteristics to a specific cell lineage. For example, endothelial cells are isolated by attaching an antibody directed against the platelet endothelial cell adhesion molecule-1 (PECAM1) such as the fluorescently-labeled PECAM1 antibodies (30884X) available from PharMingen (PharMingen, Becton Dickinson Bio Sciences, San Jose, CA, USA) as described in Levenberg, S. et al., (Endothelial cells derived from human embryonic stem cells. Proc. Natl. Acad. Sci. USA. 2002. 99: 4391-4396). Hematopoietic cells are isolated using fluorescently-labeled antibodies such as CD34-FITC, CD45-PE, CD31-PE, CD38-PE, CD90-FITC, CD117-PE, CD15-FITC, class I-FITC, all of which IgGI are available from PharMingen, CD133/1-PE (IgGI) (available from Miltenyi Biotec, Auburn, CA), and glycophorin A-PE (IgGI), available from Immunotech (Miami, FL). Live cells (i.e., without fixation) are analyzed on a FACScan (Becton Dickinson Bio Sciences) by using propidium iodide to exclude dead cells with either the PC-LYSIS or the CELLQUEST software. It will be appreciated that isolated cells can be further enriched using magnetically-labeled second antibodies and magnetic separation columns (MACS, Miltenyi) as described by Kaufman, D. S. et al., (Hematopoietic colony-forming cells derived from human embryonic stem cells. Proc. Natl. Acad. Sci. USA. 2001, 98: 10716-10721).

According to some embodiments of the invention, isolating lineage specific cells is effected by a mechanical separation of cells, tissues and/or tissue-like structures contained within the EBs.

For example, beating cardiomyocytes can be isolated from EBs as disclosed in U.S. Pat. Appl. No. 20030022367 to Xu et al. Four-day-old EBs of the present invention are transferred to gelatin-coated plates or chamber slides and are allowed to attach and differentiate. Spontaneously contracting cells, which are observed from day 8 of differentiation, are mechanically separated and collected into a 15-mL tube containing low-calcium medium or PBS. Cells are dissociated using Collagenase B digestion for 60-120 minutes at 37° C., depending on the Collagenase activity. Dissociated cells are then resuspended in a differentiation KB medium (85 mM KCl, 30 mM K₂IPO₄, 5 mM MgSO₄, 1 mM EGTA, 5 mM creatine, 20 mM glucose, 2 mM Na₂ATP, 5 mM pyruvate, and 20 mM taurine, buffered to pH 7.2, Maltsev et al., Circ. Res. 75:233, 1994) and incubated at 37° C. for 15-30 min. Following dissociation cells are seeded into chamber slides and cultured in the differentiation medium to generate single cardiomyocytes capable of beating.

It will be appreciated that the culturing conditions suitable for the differentiation and expansion of the isolated lineage specific cells include various tissue culture medium, growth factors, antibiotic, amino acids and the like and it is within the capability of one skilled in the art to determine which conditions should be applied in order to expand and differentiate particular cell types and/or cell lineages [reviewed in Fijnvandraat A C, et al., Cardiovasc Res. 2003; 58: 303-12; Sachinidis A, et al., Cardiovasc Res. 2003; 58: 278-91; Stavridis M P and Smith A G, 2003; Biochem Soc Trans. 31(Pt 1): 45-9].

According to some embodiments of the invention, isolating lineage specific cells is effected by subjecting the EBs to differentiation factors to thereby induce differentiation of the EBs into lineage specific differentiated cells. Non-limiting procedures and approaches for inducing differentiation of EBs to lineage specific cells are described below.

### Neural Precursor Cells

To differentiate the EBs of some embodiments of the invention into neural precursors, four-day-old EBs are cultured for 5-12 days in tissue culture dishes including DMEM/F-12 medium with 5 mg/ml insulin, 50 mg/ml transferrin, 30 nM selenium chloride, and 5 mg/ml fibronectin (ITSFn medium, Okabe, S. et al., 1996, Mech. Dev. 59: 89-102). The resultant neural precursors can be further transplanted to generate neural cells in vivo (Brustle, O. et al., 1997. In vitro-generated neural precursors participate in mammalian brain development. Proc. Natl. Acad. Sci. USA. 94: 14809-14814). It will be appreciated that prior to their transplantation, the neural precursors are trypsinized and triturated to single-cell suspensions in the presence of 0.1% DNase.

### Oligodendrocytes and Myelinate Cells

EBs of some embodiments of the invention can differentiate to oligodendrocytes and myelinate cells by culturing the cells in modified SATO medium, i.e., DMEM with bovine serum albumin (BSA), pyruvate, progesterone, putrescine, thyroxine, triiodothryonine, insulin, transferrin, sodium selenite, amino acids, neurotrophin 3, ciliary neurotrophic factor and Hepes (Bottenstein, J. E. & Sato, G. H., 1979, Proc. Natl. Acad. Sci. USA 76, 514-517; Raff, M. C., Miller, R. H., & Noble, M., 1983, Nature 303: 390-396]. Briefly, EBs are dissociated using 0.25% Trypsin/EDTA (5 min at 37° C.) and triturated to single cell suspensions. Suspended cells are plated in flasks containing SATO medium supplemented with 5% equine serum and 5% fetal calf serum (FCS). Following 4 days in culture, the flasks are gently shaken to suspend loosely adhering cells (primarily oligodendrocytes), while astrocytes are remained adhering to the flasks and further producing conditioned medium. Primary oligodendrocytes are transferred to new flasks containing SATO medium for additional two days. Following a total of 6 days in culture, oligospheres are either partially dissociated and resuspended in SATO medium for cell transplantation, or completely dissociated and a plated in an oligosphere-conditioned medium which is derived from the previous shaking step [Liu, S. et al., (2000). Embryonic stem cells differentiate into oligodendrocytes and myelinate in culture and after spinal cord transplantation. Proc. Natl. Acad. Sci. USA. 97: 6126-6131].

### Mast Cells

For mast cell differentiation, two-week-old EBs of some embodiments of the invention are transferred to tissue culture dishes including DMEM medium supplemented with 10% FCS, 2 mM L-glutamine, 100 units/ml penicillin, 100 mg/ml streptomycin, 20% (v/v) WEHI-3 cell-conditioned medium and 50 ng/ml recombinant rat stem cell factor (rrSCF, Tsai, M. et al., 2000. In vivo immunological function of mast cells derived from embryonic stem cells: An approach for the rapid analysis of even embryonic lethal mutations in adult mice in vivo. Proc Natl Acad Sci USA. 97: 9186-9190). Cultures are expanded weekly by transferring the cells to new flasks and replacing half of the culture medium.

### Hemato-Lymphoid Cells

To generate hemato-lymphoid cells from the EBs of some embodiments of the invention, 2-3 days-old EBs are transferred to gas-permeable culture dishes in the presence of 7.5% CO₂ and 5% O₂ using an incubator with adjustable oxygen content. Following 15 days of differentiation, cells are harvested and dissociated by gentle digestion with Collagenase (0.1 unit/mg) and Dispase (0.8 unit/mg), both are available from F. Hoffman-La Roche Ltd, Basel, Switzerland. CD45-positive cells are isolated using anti-CD45 monoclonal antibody (mAb) M1/9.3.4.HL.2 and paramagnetic microbeads (Miltenyi) conjugated to goat anti-rat immunoglobulin as described in Potocnik, A. J. et al., (Immunology Hemato-lymphoid in vivo reconstitution potential of subpopulations derived from in vitro differentiated embryonic stem cells. Proc. Natl. Acad. Sci. USA. 1997, 94: 10295-10300). The isolated CD45-positive cells can be further enriched using a single passage over a MACS column (Miltenyi).

It should be noted that EBs of some embodiments of the invention can be used to generate lineage-specific cell lines which are capable of unlimited expansion in culture.

Cell lines of some embodiments of the invention can be produced by immortalizing the EB-derived cells by methods known in the art, including, for example, expressing a telomerase gene in the cells (Wei, W. et al., 2003. Mol Cell Biol. 23: 2859-2870) or co-culturing the cells with NIH 3T3 hph-HOX11 retroviral producer cells (Hawley, R. G. et al., 1994. Oncogene 9: 1-12).

As mentioned above, lineage specific cells can be also obtained by directly inducing the expanded, undifferentiated pluripotent stem cells such as ESCs or iPS cells to culturing conditions suitable for the differentiation of specific cell lineage.

According to an aspect of some embodiments of the invention there is provided a method of differentiating mammalian livestock pluripotent stem cells. The method is performed by (a) culturing the mammalian livestock pluripotent stem cells according to the method of some embodiments of the invention, to thereby obtain an expanded population of mammalian livestock pluripotent stem cells in an undifferentiated state, and (b) culturing the expanded population of mammalian livestock pluripotent stem cells in an undifferentiated state under conditions devoid of the differentiation inhibiting agent which allow differentiation of the mammalian livestock pluripotent stem cells, thereby differentiating the mammalian livestock pluripotent stem cells.

According to some embodiments of the invention the culturing in steps (a) and (b) is performed in a suspension culture.

According to some embodiments of the invention the culturing in the suspension culture is without adherence to a substrate.

The mammalian livestock pluripotent stem cells of some embodiments of the invention can be induced to differentiation into various cell lineages and cell types.

According to some embodiments of the invention the conditions comprise culturing the cells in a culture medium suitable for differentiating the mammalian livestock undifferentiated stem cells into muscle cells.

Differentiation into cardiomyocytes—Pluripotent stem cells can be induced to differentiation into cardiomyocytes using various known methods such as those described in P. W. Burridge et al., (2014; Nat Methods. 11: 855-860; “Chemically defined generation of human cardiomycytes”); I. Batalov et al., (2015; Biomarker Insights 2015:10(S1); “Differentiation of Cardiomycytes from Human Pluripotent Stem Cells Using Monolayer Culture”); and P. W. Burridge et al. 2013 (Chapter 12 In: Methods in Molecular Biology 997; Uma Lakshmipathy and Mohan C. Vemuri Editors; Pluripotent Stem Cells, Methods and Protocols; “Highly Efficient Directed Differentiation of Human Induced Pluripotent Stem Cells into Cardiomyocytes”), each of which is fully incorporated herein by reference in its entirety. For example, for cardiomyocyte differentiation the pluripotent stem cells can be cultured in a conditioned medium, allowing formation of embryoid bodies (EBs), which can then be exposed to a serum containing medium (e.g., fetal bovine serum) for adhesion and formation of contracting cardiomyocytes.

Differentiation into Smooth muscle cells—Pluripotent stem cells can be induced to differentiation into smooth muscle cells using various known methods, such as using multipotent vasculogenic pericytes, which can successfully differentiate into smooth muscle cells, essentially as described in Dar A., et al., 2012 (Circulation. 125: 87-99; “Multipotent Vasculogenic Pericytes From Human Pluripotent Stem Cells Promote Recovery of Murine Ischemic Limb”), which is fully incorporated herein by reference in its entirety. Briefly, the pluripotent stem cells undergo spontaneous differentiation into EBs and cells of the EBs which are CD105⁺/CD90⁺/CD73⁺/CD31⁻ multipotent clonogenic mesodermal precursors can be isolated by MACS MicroBeads and give rise to pericytes, which can further proliferate and further differentiated into smooth muscle cells.

Additionally or alternatively, pluripotent stem cells can be cultured in a chemically defined culture medium comprising inhibitors of phosphoinositide 3-kinase (PI3K) and glycogen synthase kinase 3b(GSK3b) and the addition of bone morphogenic protein 4 (BMP4) and fibroblast growth factor 2 (FGF2), to successfully convert up to about 60% of the cells into the myogenic program by day 36 as indicated by MYOG+ cell populations, essentially as described in ELLIOT W. SWARTZ, et al., 2016 (“A Novel Protocol for Directed Differentiation of C9orf72-AssociatedHumanInduced Pluripotent Stem Cells Into Contractile Skeletal Myotubes”; STEM CELLS TRANSLATIONAL MEDICINE 2016; 5:1461-1472), which is fully incorporated herein by reference in its entirety.

Additional suitable methods of inducing differentiation of pluripotent stem cells into muscle cells are described in Jerome Chal et al., 2016 (“Generation of human muscle fibers and satellite-like cells from human pluripotent stem cells in vitro”; Nature protocols; VOL. 11: 1833-1850); Nunnapas Jiwlawat et al., 2018 (“Current Progress and Challenges for Skeletal Muscle Differentiation from Human Pluripotent Stem Cells Using Transgene-Free Approaches”; Stem Cells International, Volume 2018, pp: 1-18), each of which is fully incorporated herein by reference in its entirety.

According to some embodiments of the invention the conditions comprise culturing the cells in a culture medium suitable for differentiating the mammalian livestock undifferentiated stem cells into blood cells.

Differentiation into red blood cells—Pluripotent stem cells can be induced to differentiation into hematopoietic cells, such as red blood cells using various protocols.

For example, differentiation into hematopoietic cells can be achieved via differentiation of the pluripotent stem cells into embryoid bodies (EBs).

Pluripotent stem cells can be induced to differentiation to hematopoietic cells by spontaneous differentiation into embryoid bodies (EBs), essentially as described in H. Lapillonne, et al., 2010 [haematologica, 95(10): 1651-1659; “Red blood cell generation from human induced pluripotent stem cells: perspectives for transfusion medicine” ], which is fully incorporated herewith in its entirety. Briefly, differentiation into EBs is performed in the presence of a culture medium such as Iscove's modified Dulbecco's medium-glutamax containing human plasma in the presence of stem cell factor (SCF, e.g., about 100 ng/mL), thrombopoietin (TPO, e.g., about 100 ng/mL), FLT3 ligand (e.g., about 100 ng/mL), recombinant human bone morphogenetic protein 4 (BMP4; e.g., about 10 ng/mL), recombinant human vascular endothelial growth factor (VEGF-A165; e.g., about 5 ng/mL), interleukin-3 (IL-3; e.g., about 5 ng/mL), interleukin-6 (IL-6; e.g., about 5 ng/mL) and erythropoietin (Epo; e.g., about 3 U/mL). Following about 20 days in culture the resulting embryoid bodies contain cells having early erythroid commitment. The cells of the EBs are then dissociated into single cells and further cultured in a culture medium containing plasma (e.g., about 10%), insulin (e.g., about 10 μg/ml) and heparin (e.g., about 3 U/mL) and additional factors such as SCF (e.g., about 100 ng/mL), IL-3 (e.g., about 5 ng/mL) and Epo (e.g., about 3 U/mL). Following 8 days in culture the medium is replaced with a culture medium supplemented with SCF (e.g., about 100 ng/mL) and Epo (e.g., about 3 U/mL) for additional 3 days. From day 11 to 25 the cells can be cultured in a medium supplemented with Epo (3 U/mL). This protocol can result in definitive erythrocytes capable of maturation up to enucleated red blood cells containing fetal hemoglobin in a functional tetrameric form.

Alternatively, pluripotent stem cells can be directly differentiated into definite erythroblasts, essentially as described in Bin Mao et al. (2016, Stem Cell Reports, Vol. 7, pp 869-883), which is fully incorporated herein by reference in its entirety. Briefly, pluripotent stem cells which are cultured on a two-dimensional matrix or on feeder cells can be induced to differentiation into hematopoietic lineage by replacing the culture medium from an hPSCs maintenance medium to a hematopoiesis-inducing medium. For example, the hematopoiesis-inducing medium can be an Iscove's modified Dulbecco's medium (IMDM) supplemented with fetal bovine serum (FBS; e.g., about 10%) (e.g., Hyclone), 1% non-essential amino acids, ascorbic acid (e.g., about 50 mg/mL), and VEGF (Vascular endothelial growth factor; e.g., about 20 ng/mL), and culturing can be for a culturing period of about 10-12 days so as to form hematopoietic and erythroid progenitors. At days 10-12 the co-culture can be harvested and transferred to an ultra-low attachment plate with serum-free expansion medium supplemented with stem cell factor (SCF; e.g., about 100 ng/mL), interleukin-6 (IL-6; e.g., about 100 ng/mL), interleukin-3 (IL-3; e.g., about 5 ng/mL), fetal liver (e.g., about 10 ng/mL), thrombopoietin (TPO; e.g., about 10 ng/mL), erythropoietin (EPO; e.g., about 4 IU/mL), and VEGF (e.g., about 20 ng/mL) for 6 days, following which the cells are cultured for additional 7-8 days in a serum-free medium supplemented with stem cell factor, interleukin-3 (IL-3) and erythropoietin. Finally for maturation of the erythroblasts, the cells are cultured for about 1-2 weeks in serum-free RBC medium supplemented erythropoietin (EPO) essentially as described in Giarratana, M. C., 2005 (Nat. Biotechnol. 23, 69-74), which is fully incorporated herewith in its entirety. It is noted that the mature erythroblasts (derived from pluripotent stem cells) can be identified by the GPA+CD36^{low}/⁺ which express higher levels of beta-globin along with a gradual loss of mesodermal and endothelial properties, and terminally suppressed CD36.

Additionally or alternatively it is noted that once CD34+ cells are obtained or isolated, enucleated red blood cells can be obtained under feeder-free culture conditions essentially as described in Kenichi Miharada et al., 2006 (“Efficient Enucleation of Erythroblasts Differentiated in Vitro From Hematopoietic Stem and Progenitor Cells”; Nat. Biotechnol. 24(10):1255-6), which is fully incorporated herein by reference in its entirety. Briefly, CD34+ cells are cultured in a culture medium containing stem cell factor (SCF), eruthropoietin (EPO), interleukin-3 (IL-3), vascular endothelial growth factor (VEGF) and insulin-like growth factor-II (IGF-II) for the first passage and then in a medium supplemented with only SCF and EPO for passages II and III, to thereby obtain about 77% of nucleated red blood cells.

According to some embodiments of the invention the conditions comprise culturing the cells in a culture medium suitable for differentiating the mammalian livestock undifferentiated stem cells into adipogenic (e.g., fat) cells.

Differentiation into adipogenic cell lineage—It is well known in the art that pluripotent stem cells can be induced to differentiation into the adipogenic lineage by direct induction in the presence of effective amounts of adipogenic differentiation agents. For example, direct differentiation can be achieved by culturing the pluripotent stem cells in the presence of a bone morphogenic protein 4 (BMP4) essentially as described in Qi-Qun Tang, 2004 [Proc. Natl. Acad. Sci. U.S.A. 101(26): 9607-9611 “Commitment of C3H10T1/2 pluripotent stem cells to the adipocyte lineage” ]. Additionally or alternatively, pluripotent stem cells can be differentiated into adipogenic cells via embryoid bodies (EBs) differentiation. For example, 10-day old EBs can be plated on gelatin-coated plates with medium (e.g., DMEM/F12) comprising 20% KSR (knockout serum replacement), and following additional 10 days the outgrowth are cultured in a medium containing DMEM/F12 and 10% KSR supplemented with IBMX (1-Methyl-3-Isobutylxanthine; e.g., at a concentration of 0.5 mM), dexamethasone (e.g., 0.25 μM), T3 (e.g., 0.2 nM), insulin (e.g., 1 μg/ml), and Rosiglitazone (e.g., 1 μM), essentially as described in Tala Mohsen-Kanson et al., 2014 (Stem Cells, 32: 1459-1467), which is fully incorporated herein by reference.

As used herein the phrase “adipogenic differentiation agent” refers to a substance e.g., hormone and/or a chemical agent which when added to pluripotent stem cells in an in-vitro culture results in induction of differentiation of the cells towards the adipogenic cell lineage, ultimately resulting in the generation of adipocytes.

According to some embodiments of the invention, the adipogenic differentiation agent induces differentiation towards adipogenic lineage of pluripotent stem cells which are cultured in a two-dimensional culture system (e.g., on a matrix or on feeder cell layer(s)).

Non-limiting examples of known adipogenic differentiation agents include, but are not limited to, IBMX (1-Methyl-3-Isobutylxanthine, or 3-isobutyl-1-methylxanthine, which are interchangeably used herein), hydrocortisone, dexamethasone, BMP (bone morphogenic protein), T3 (triiodothyronine), indomethacin and fatty acids such as monounsaturated omega5 (e.g., Myristoleic acid), monounsaturated omega7 (e.g., Palmitoleic acid), monounsaturated omega 9 (e.g., Erucic acid, Elaidic acid, Oleic acid) or branched fatty acids (e.g., Phytanic acid and Pristanic acid) essentially as described in F. Mehta et al 2019 Sissel Beate Ronning (ed.), Myogenesis: Methods and Protocols, Methods in Molecular Biology, vol. 1889, Springer Science+Business Media, LLC, part of Springer Nature 2019.

Following are exemplary effective concentration ranges suitable for inducing adipogenic differentiation of pluripotent stem cell such as human ESCs or iPSCs. An adipogenic differentiation medium may comprise 0.01-1 mM of 3-isobutyl-1-methylxanthine, 0.1-10 μM of hydrocortisone, 0.01-1 mM of indomethacin, 0.4-0.6 mM IBMX, 0.2-0.3 μM dexamethasone, 0.15-0.3 nM T3, 1-2 μg/ml insulin, and 1-2 μM Rosiglitazone.

According to some embodiments of the invention, mammalian livestock pluripotent stem cells which are derived from a delayed blastocyst can spontaneously differentiate into adipogenic lineage without the addition of an adipogenic differentiation agent.

According to some embodiments of the invention the conditions comprise culturing the cells in a culture medium suitable for differentiating the mammalian livestock undifferentiated stem cells into connective tissue cells.

Differentiation into cartilage cells—Pluripotent stem cells can be induced to differentiation into cartilage cells via formation of embryoid bodies, e.g., essentially as described in Sergey P. Medvedev et al., 2011 (“Human Induced Pluripotent Stem Cells Derived from Fetal Neural Stem Cells Successfully Undergo Directed Differentiation into Cartilage”; STEM CELLS AND DEVELOPMENT, Volume 20, Number 6: 1099-1112), which is fully incorporated herein by reference in its entirety. Briefly, pluripotent stem cells are allowed to spontaneously differentiate into embryoid bodies for 8-15 days. For directed chondrogenic differentiation, the embryoid bodies can be further cultivated for 21 days in a chondrogenic medium comprising DMEM, supplemented with bovine serum (e.g., about 5%), dexamethasone (e.g., about 10 nM), ascorbic acid (e.g., about 50 μg/mL), L-proline (e.g., about 40 μg/mL), transforming growth factor b3 (TGFβ3; e.g., about 10 ng/mL) and bone morphogenetic protein-2 (BMP2; e.g., about 10 ng/mL). For a further cartilage self-assembly, the EBs can be disaggregated (e.g., using trypsin), and further transferred to coated 96-well plates (e.g., coated with agarose), at a density of 10⁵ cells per well and further cultured in the same medium.

Additionally or alternatively, pluripotent stem cells can be directly differentiated into chondrocytes by plating the cells on a matrix in the presence of a chondrogenic-inducing culture medium, using various protocols, for example, as reviewed in Michal Lach et al., 2014. Journal of Tissue Engineering Volume 5: 1-9, which is fully incorporated herein by reference in its entirety. For example, pluripotent stem cells can be cultured on a matrix in a medium supplemented with various growth factors such as WNT-3a, activin, follistatin, BMP4, fibroblast growth factor 2 (FGF2), growth and differentiation factor 5 (GDF5) and neurotrophin 4 (NT4), essentially as described in Oldershaw R A, et al. 2010 (“Directed differentiation of human embryonic stem cells toward chondrocytes”; Nat Biotechnol 28(11): 1187-1194), which is fully incorporated herein by reference in its entirety.

Additionally or alternatively, for differentiation into chondrocyte-like cells the pluripotent stem cells can be cultured in a medium comprising only six growth factors WNT-3a, activin, follistatin, BMP4, fibroblast growth factor 2 (FGF2), and growth and differentiation factor 5 (GDF5) essentially as described in Yang S-L, et al. 2012 (“Compound screening platform using human induced pluripotent stem cells to identify small molecules that promote chondrogenesis”. Protein Cell, 3(12): 934-942), which is fully incorporated herein by reference in its entirety. These protocols can result in differentiation into chondrocyte-like cells with high COL2A1 (Collagen type II, alpha 1) and SRY (sex determining region Y)-box 9 (SOX9) expression and decreased pluripotent marker expression compared to control cell lines.

According to some embodiments of the invention the pluripotent stem cells can be differentiated to generate mesenchymal stromal cells.

Mesenchymal stromal cells which are CD73-positive and SSEA-4-negative can be generated from pluripotent stem cells by mechanically increasing the fraction of fibroblast-like differentiated cells formed in cultures of pluripotent stem cells, essentially as described in Trivedi P and Hematti P. Exp Hematol. 2008, 36(3):350-9. Briefly, to induce differentiation of pluripotent stem cells the intervals between medium changes are increased to 3-5 days, and the cells at the periphery of the ESC colonies become spindle-shaped fibroblast-looking cells. After 9-10 days under these conditions when about 40-50% of the cells in the culture acquire the fibroblast-looking appearance, the undifferentiated portions of pluripotent stem cells colonies are physically removed and the remaining differentiated cells are passaged to new culture plates under the same conditions.

According to some embodiments of the invention the pluripotent stem cells can be differentiated to generate dopaminergic (DA) neurons.

To induce differentiation of pluripotent stem cells into dopaminergic (DA) neurons, the cells can be co-cultured with the mouse stromal cell lines PA6 or MS5, or can be cultured with a combination of stromal cell-derived factor 1 (SDF-1/CXCL12), pleiotrophin (PTN), insulin-like growth factor 2 (IGF2) and ephrin B1 (EFNB1) essentially as described in Vazin T, et al., PLoS One. 2009 Aug. 12; 4(8):e6606; and in Elkabetz Y., et al., Genes Dev. 2008 January 15; 22: 152-165.

According to some embodiments of the invention the pluripotent stem cells can be differentiated to generate mesencephalic dopamine (mesDA) neurons.

To generate mesencephalic dopamine (mesDA) neurons, pluripotent stem cells can be genetically modified to express the transcription factor Lmx1a (e.g., using a lentiviral vector with the PGK promoter and Lmx1a) essentially as described in Friling S., et al., Proc Natl Acad Sci USA. 2009, 106: 7613-7618.

According to some embodiments of the invention the pluripotent stem cells can be differentiated to generate lung epithelium (type II pneumocytes).

To generate lung epithelium (type II pneumocytes) from pluripotent stem cells, the pluripotent stem cells can be cultured in the presence of a commercially available cell culture medium (Small Airway Growth Medium; Cambrex, College Park, MD), or alternatively, in the presence of a conditioned medium collected from a pneumocyte cell line (e.g., the A549 human lung adenocarcinoma cell line) as described in Rippon H J., et al., Proc Am Thorac Soc. 2008; 5: 717-722.

According to some embodiments of the invention the pluripotent stem cells can be differentiated to generate neural cells.

To induce differentiation of pluripotent stem cells into neural cells, the pluripotent stem cells can be cultured for about 5 days in the presence of a serum replacement medium supplemented with TGF-b inhibitor (SB431542, Tocris; e.g., 10 nM) and Noggin (R&D; e.g., 500 ng/ml), following which the cells are cultured with increasing amounts (e.g., 25%, 50%, 75%, changed every two days) of N2 medium (Li X J., et al., Nat Biotechnol. 2005, 23:215-21) in the presence of 500 ng/mL Noggin, essentially as described in Chambers S M., et al., Nat Biotechnol. 2009, 27: 275-280.

It should be noted that cells which are differentiated from the mammalian livestock pluripotent stem cells of some embodiments of the invention can be incorporated into a food product.

According to an aspect of some embodiments of the invention there is provided a method of preparing food product, comprising combining differentiated mammalian livestock cells resultant from the method of some embodiments of the invention with a food product, thereby preparing the food product.

According to an aspect of some embodiments of the invention there is provided food product comprising differentiated mammalian livestock cells resultant from the method of some embodiments of the invention.

According to some embodiments of the invention, the food product comprises a cultured meat or cultured cells which can be combined with other substances to result in cultured meat.

As used herein the term “cultured meat” refers to in-vitro cultured animal cells processed to impart an organoleptic sensation and texture of meat.

The cultured meat product may include a variety of cells, including but not limited to adipocytes, muscle cells, blood cells, cartilage cells, bone cells, connective tissue cells, fibroblasts and/or cardiomyocytes.

According to some embodiments of the invention, the in vitro cultured animal cells are mammalian livestock cells.

According to some embodiments of the invention, the in vitro cultured animal cells are bovine cells (though other cells can be included e.g., fish, porcine, avian etc).

According to some embodiments of the invention, the in vitro cultured animal cells are adipocytes which are obtained by spontaneous differentiation of the mammalian livestock pluripotent stem cells of some embodiments of the invention.

According to some embodiments of the invention, the cultured meat is substantially free from any harmful microbial or parasitic contamination.

It should be noted that the fattier meat is generally tastier, but a greater fat content may pose a greater risk of adverse health consequences such as heart disease.

According to some embodiments of the invention, the cultured meat includes a ratio of muscle to fat cells that can be controlled to produce a meat product with optimal flavor and health effects. For example, such a ratio can be controlled by initial seeding of the desired cells in a culture or by controlling the differentiation of the mammalian livestock pluripotent stem cells into muscle, cartilage, blood or fat cells.

Differentiation may occur on supporting layers to support the structure and/or texture of the cultured meat.

According to some embodiments of the invention, aseptic techniques may be used to culture the cells resulting in meat products that are substantially free from harmful microbes such as bacteria, fungi, viruses, prions, protozoa, or any combination of the above. Harmful microbes may include pathogenic type microorganisms such as salmonella, campylobacter, E. coli 0156:H7, etc. Aseptic techniques may also be employed in packaging the meat products as they come off the biological production line. Such quality assurance may be monitored by standard assays for microorganisms or chemicals that are already known in the art. “Substantially free” means that the concentration of microbes or parasites is below a clinically significant level of contamination, i.e., below a level wherein ingestion would lead to disease or adverse health conditions.

According to some embodiments of the invention, other nutrients such as vitamins that are normally lacking in meat products from whole animals may be added to increase the nutritional value of the meat. This may be achieved either through straight addition of the nutrients to the growth medium or through genetic engineering techniques. For example, the gene or genes for enzymes responsible for the biosynthesis of a particular vitamin, such as Vitamin D, A, or the different Vitamin B complexes, may be transfected in the cultured muscle cells to produce the particular vitamin.

According to some embodiments of the invention, the meat product derived from the cultured cells in vitro may include different derivatives of meat products. These derivatives may be prepared, for example, by grounding or shredding the tissues grown in vitro and mixed with appropriate seasoning to make meatballs, fishballs, hamburger patties, etc. The derivatives may also be prepared from layers of tissues cut and spiced into, for example, beef jerky, ham, bologna, salami, etc. Thus, the meat products of the present invention may be used to generate any kind of food product originating from the meat of an animal.

### Teratomas

The pluripotent capacity of the pluripotent stem cells of some embodiments of the invention can also be confirmed by injecting the cells into SCID mice [Evans M J and Kaufman M (1983). Pluripotential cells grown directly from normal mouse embryos. Cancer Surv. 2: 185-208], which upon injection form teratomas. Teratomas are fixed using 4% paraformaldehyde and histologically examined for the three germ layers (i.e., endoderm, mesoderm and ectoderm).

In addition to monitoring a differentiation state, stem cells are often also being monitored for karyotype, in order to verify cytological euploidity, wherein all chromosomes are present and not detectably altered during culturing. Cultured stem cells can be karyotyped using a standard 30 Giemsa staining and compared to published karyotypes of the corresponding species.

As mentioned, any of the proteinaceous factors used in the culture medium of the present invention (e.g., the bFGF, IL6RIL6 chimera, WNT3a, LIF) can be recombinantly expressed or biochemically synthesized. In addition, naturally occurring proteinaceous factors such as bFGF, WNT3a, LIF can be purified from biological samples (e.g., from human serum, cell cultures) using methods well known in the art. It should be noted that for the preparation of xeno-free culture medium the proteinaceous factor is preferably recombinantly expressed.

Biochemical synthesis of the proteinaceous factors of the present invention can be performed using standard solid phase techniques. These methods include exclusive solid phase synthesis, partial solid phase synthesis methods, fragment condensation and classical solution synthesis.

Recombinant expression of the proteinaceous factors of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463. Specifically, the IL6RIL6 chimera can be generated as described in PCT publication WO 99/02552 to Revel M., et al. and Chebath J, et al., 1997, which are fully incorporated herein by reference.

As mentioned, the method of some embodiments of the invention employs culturing the mammalian (e.g., livestock) pluripotent stem cells on feeder cell layers or on feeder cell-free culture systems.

Following are exemplary, non-limiting descriptions of feeder cell layers.

Mouse feeder layers—The most common method for culturing pluripotent stem cells is based on mouse embryonic fibroblasts (MEF) as a feeder cell layer supplemented with tissue culture medium containing serum or leukemia inhibitor factor (LIF) which supports the proliferation and the pluripotency of the pluripotent stem cells [Thomson J A, Itskovitz-Eldor J, Shapiro S S, Waknitz M A, Swiergiel J J, Marshall V S, Jones J M. (1998). Embryonic stem cell lines derived from human blastocysts. Science 282: 1145-7; Reubinoff B E, Pera M F, Fong C, Trounson A, Bongso A. (2000). Embryonic stem cell lines from human blastocysts: somatic differentiation in vitro. Nat. Biotechnol. 18: 399-404]. MEF cells are derived from day 12-13 mouse embryos in medium supplemented with fetal bovine serum. Under these conditions mouse ES cells can be maintained in culture as pluripotent stem cells, preserving their phenotypical and functional characteristics. It should be noted that the use of feeder cells substantially increases the cost of production. Additionally, the feeder cells are metabolically inactivated to keep them from outgrowing the stem cells, hence it is necessary to have fresh feeder cells for each splitting of pluripotent stem cell culture.

Pluripotent stem cells can also be cultured on MEF under serum-free conditions using serum replacement supplemented with basic fibroblast growth factor (bFGF) [Amit M, Carpenter M K, Inokuma M S, Chiu C P, Harris C P, Waknitz M A, Itskovitz-Eldor J, Thomson J A. (2000). Clonally derived human embryonic stem cell lines maintain pluripotency and proliferative potential for prolonged periods of culture. Dev. Biol. 227: 271-8]. Under these conditions the cloning efficiency of ES cells is 4 times higher than under fetal bovine serum. In addition, following 6 months of culturing under serum replacement the ES cells still maintain their pluripotency as indicated by their ability to form teratomas which contain all three embryonic germ layers. Although this system uses a better-defined culture conditions, the presence of mouse cells in the culture may expose the pluripotent stem cell culture to mouse pathogens which restricts their use in cell-based therapy.

Human embryonic fibroblasts or adult fallopian epithelial cells as feeder cell layers—Embryonic stem cells can be grown and maintained using human embryonic fibroblasts or adult fallopian epithelial cells. When grown on these human feeder cells the embryonic stem cells exhibit normal karyotypes, present alkaline phosphatase activity, express Oct-4 and other embryonic cell surface markers including SSEA-3, SSEA-4, TRA-1-60, and GCTM-2, form teratomas in vivo, and retain all key morphological characteristics [Richards M, Fong C Y, Chan W K, Wong P C, Bongso A. (2002). Human feeders support prolonged undifferentiated growth of human inner cell masses and embryonic stem cells. Nat. Biotechnol. 20: 933-6].

Foreskin feeder layers—Embryonic stem cells can be cultured on human foreskin feeder layer as disclosed in U.S. patent application Ser. No. 10/368,045. Foreskin derived feeder cell layers consist of a complete animal-free environment suitable for culturing embryonic stem cells. In addition, foreskin cells can be maintained in culture for as long as 42 passages since their derivation, providing the embryonic stem cells with a relatively constant environment. Under these conditions the embryonic stem cells were found to be functionally indistinct from cells grown with alternate protocols (e.g., MEF). Following differentiation, embryonic stem cells expressed genes associated with all three embryonal germ layers, in vitro, and formed teratomas in vivo, consisting of tissue arising from all three germ layers. In addition, unlike human fallopian epithelial cells or human embryonic fibroblasts, human embryonic stem cells cultured on foreskin feeder layers were maintained in culture in a pluripotent and undifferentiated state for at least 87 passages. However, although foreskin cells can be maintained in culture for long periods (i.e., 42 passages), the foreskin culture system is not well-defined due to differences between separate batches. In addition, human feeder layer-based culture systems would still require the simultaneous growth of both feeder layers and hES cells. Therefore, feeder-free culturing systems have been developed.

As described, the pluripotent stem cells of some embodiments of the invention can be cultured and maintained in an undifferentiated state for extended periods of time, e.g., for at least 5 passages or more (e.g., for more than 10, 15, 20, 25, 30, 35, 40 passages) while being cultured on feeder-free culture systems.

Pluripotent stem cells can be grown on a solid surface such as an extracellular matrix in the presence of a culture medium. Unlike feeder-based cultures which require the simultaneous growth of feeder cells and stem cells and which may result in mixed cell populations, pluripotent stem cells grown on feeder-free systems are easily separated from the surface. The culture medium used for growing the stem cells contains factors that effectively inhibit differentiation and promote their growth (e.g., the differentiation inhibitory factor(s) described herein).

Commonly used feeder-free culturing systems utilize an animal-based matrix (e.g., Matrigel®) supplemented with mouse or bovine serum, or with MEF conditioned medium [Xu C, et al. (2001). Feeder-free growth of undifferentiated human embryonic stem cells. Nat Biotechnol. 19: 971-4] which present the risk of animal pathogen cross-transfer to other species (e.g., human) pluripotent stem cells.

The extracellular matrix can be composed of components derived from basement membrane and/or extracellular matrix components that form part of adhesion molecule receptor-ligand couplings. MATRIGEL® (Becton Dickinson, USA) is one example of a commercially available matrix which is suitable for use with the present invention. MATRIGEL® is a soluble preparation from Engelbreth-Holm-Swarm tumor cells that gels at room temperature to form a reconstituted basement membrane; MATRIGEL® is also available as a growth factor reduced preparation. Other extracellular matrix components and component mixtures which are suitable for use with the present invention include foreskin matrix, laminin matrix, fibronectin matrix, proteoglycan matrix, entactin matrix, heparan sulfate matrix, collagen matrix and the like, alone or in various combinations thereof.

According to some embodiments of the invention, the feeder-free matrix is selected from the group consisting of a Matrigel™ matrix, a fibronectin matrix, a laminin matrix, collagen matrix, Elastin matrix, and a vitronectin matrix.

According to some embodiments of the invention the matrix is xeno-free.

In cases where complete xeno-free culturing conditions are desired, the matrix is preferably derived from the same source of the embryo, e.g., a mammalian livestock, e.g., a bovine, or can be synthesized using recombinant techniques. Such matrices include, for example, recombinant fibronectin, recombinant laminin, a synthetic fibronectin matrix, Vitronectin matrix, and/or a collagen matrix. A synthetic fibronectin matrix can be obtained from Sigma, St. Louis, MO, USA.

The pluripotent stem cells of some embodiments of the invention, or the cells differentiated therefrom (e.g., adipocytes, muscle cells, blood cells, cartilage cells, bone cells, connective tissue cells, fibroblasts and/or cardiomyocytes) can be identified using various expression markers characterizing these cells. The expression markers can be identified on the RNA or protein level.

Methods of detecting the expression level of RNA include, but are not limited to Northern Blot analysis, RT-PCR analysis, RNA in situ hybridization stain, In situ RT-PCR stain, DNA microarrays/DNA chips, and Oligonucleotide microarray.

Methods of detecting expression and/or activity of proteins include, but are not limited to Enzyme linked immunosorbent assay (ELISA), Western blot, Radio-immunoassay (RIA), Fluorescence activated cell sorting adipocytes, muscle cells, blood cells, cartilage cells, bone cells, connective tissue cells, fibroblasts and/or cardiomyocytes (FACS), Immunohistochemical analysis, and In situ activity assay.

As used herein the term “about” refers to ±10%.

According to some embodiments of the invention the term “about” refers to ±9%, ±8%, ±7%, ±6%, ±5%, ±4%, ±3%, ±2%, 1%, ±0.5%, ±0.1%, or ±0.01%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 15 is expressed in a DNA sequence format (e.g., reciting T for thymine), but it can refer to either a DNA sequence that corresponds to an WNT3A nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL METHODS

### Cell Lines:

Pluripotent Stem Cells (PSCs) which are derived from delayed Bovine blastocysts (BVN1, BVN2, BVN5 and BVN6), Bovine ESCs (BVN3 and BVN4) and induced PSCs (iPSC) from Bovine fetal tissue and from endometrium epithelial cells (iBVN1.4, iBVN1.14 and iBVN1.15 respectively) were used.

Derivation of bovine PSC lines from delayed blastocytes: After zona pellucida digestion by Tyrode's acidic solution (Sigma Aldrich, St Louis, MO, USA) the exposed blastocysts were plated. There are two plating possibilities: (i) on feeder layer, such as mitotically inactivated mouse embryonic fibroblasts (MEFs) or mitotically inactivated foreskin fibroblasts, (ii) on suitable matrix (Matrigel™ matrix, Fibronectin, Laminin, Vitronectin, or other commercial extra-cellular matrices. The embryos were attached to the surface using a 27 g needle, a pulled Pasteur Pipette, by covering the embryo by a drop of a suitable matrix, or by being left overnight till the embryo spontaneously attached to the surface. Attached blastocysts were cultured on MEFs as whole embryos for 7-21 days post fertilization until a large cyst was developed. If needed due to the MEF or matrix quality, the embryos were transferred in whole to new MEF-covered plates using 27 gouge syringe needles, leaving a few of the surrounding fibroblasts behind. After the embryo developed a cyst, a disc-like structure was isolated from it and plated separately on a fresh MEF or matrix-covered plate. Cells with stem cell morphology (small cells with large nucleus) were passaged mechanically. After a few passages (e.g., about 4-6 passages), when a homogenous culture was achieved, the cells were passaged routinely every five to ten days using 1 mg/ml type IV collagenase (Gibco Invitrogen corporation products, San Diego, CA, USA).

Derivation of Bovine iPSC Lines:

Bovine iPSC cells were generated from bovine fetal cells or bovine endometrium cells. Bovine fetal cells were cultured using “medium X” (described below, with DMEM\12 as a basal medium). Bovine Endometrium cells were cultured using “medium S” (described below). Before reprograming, the Bovine Endometrium cells were plated using “medium X” (described below, with DMEM\12 as a basal medium). Cells from Passage 7 were cultured for 4-7 days before reprograming.

Reprograming was preformed using a reprogramming vector comprising the Oct3/4, Sox2, cMyc, and Klf4 genes. Suitable vectors can be found in commercially available kits such as Epi5 episomal iPSCs kit (Thrmo-Fisher), Simplicon RNA reprograming Kit (Merck-Millipore), Stemcca kit (Merck-Millipore), Stemgent stemRNA 3ed reprograming kit (Reprocelll) or CytoTune™ kit (Life Technology), each of which can be used according to manufacturer's instructions. Following about 10 days, colonies of pluripotent stem cells were isolated by picking the colonies with a using a pipette tip while viewing the cell culture under a binocular. The isolated colonies were cultured on mouse embryonic fibroblasts (MEFs) in the presence of the IL6RIL6 chimera medium (with 50 ng/ml bFGF) as described hereinbelow.

For example, iBVN 1.14 p7+23 is an induced PSC line from bovine, which was derived from an embryonic mesenchymal bovine cell at passage 7 (before reprogramming), and was cultured as an induced PSC for additional 23 passages.

### Culture Media

Medium X: This medium consists of 80% basal medium which is either DMEM\12 (Biological Industries, Bet Ha'emek, Israel) or KO-DMEM (Gibco Life Technology), and supplemented with 20% defined fetal bovine serum (defined FBS) (HyClone, Utah, USA), 1 mM L-glutamine, 0.1 mM β-mercaptoethanol, and 1% non-essential amino acid stock (all from Gibco Invitrogen corporation products, San Diego, CA, USA products).

Medium S: This medium consisting of 90% DMEM (Biological Industries, Bet Ha'emek; Israel) and supplemented with 10% defined FBS (HyClone, Utah, USA), and 1 mM L-glutamine (from Gibco Invitrogen corporation products, San Diego, CA, USA products).

The basal media used in the following culture media include low concentrations of KO-serum replacement (as indicated in the Drawings or below) or low concentrations of ITS (insulin, transferrin and selenium), fatty acids, ascorbic acid and bovine serum albumin as described below. These basal media along with the added factors were found capable of supporting the undifferentiated growth of livestock pluripotent stem cells which are cultured on feeder cell layers, or in the absence of feeder cell support such as in 3-dimensional (3-D) suspension cultures without substrate adherence for at least 5 passages.

“Basal medium-1”: Basal medium of DMEM/F12 (or KO-DMEM) at a concentration of 80-99.9% v/v supplemented with KO-serum replacement (Gibco Life Technology) as indicated below and in the Drawings (e.g., between 0.1%-20% volume/volume KoSR), 1 mM L-glutamine, 0.1 mM β-mercaptoethanol, 1% (v/v) non-essential amino acid stock, and growth factors as described below. It is noted that the concentration of the basal medium depends on the concentration of KoSR used, such that 100% volume is achieved. For example, when 5% KoSR is used then the basal medium DMEM/F12 (or KO-DMEM) is provided at a concentration of 95% v/v. When 1% KoSR is used then the basal medium DMEM/F12 (or KO-DMEM) is provided at a concentration of 99% v/v. confirmed

Chimera medium (with 50 ng/ml bFGF): basal medium-1 supplemented with 100 μg/ml IL6RIL6 chimera (R&D Systems) and 50 ng/ml basic fibroblast growth factor (bFGF) (All products but the chimera are from Gibco Invitrogen corporation products, San Diego, CA, USA).

Chimera medium (with 10 ng/ml bFGF): basal medium-1 supplemented with 100 pg\ml IL6RIL6 chimera (R&D Systems) and 10 ng/ml basic fibroblast growth factor (bFGF) (All products but the chimera are from Gibco Invitrogen corporation products, San Diego, CA, USA).

GP130 agonist based medium: basal medium-1 supplemented with IL6 (concentration of 100 ng/ml), IL11 (concentration of 1 ng/ml), LIF (concentration of 3000 U/ml), or CNTF (concentration of 1 ng/ml), along with basic fibroblast growth factor (bFGF) at concentration between 10-50 ng/ml.

LIF medium, basal medium-1 supplemented with 3000 U/ml (units per milliliter) leukemia inhibitory factor (LIF) (PeproTech) and 50 ng/ml basic fibroblast growth factor (bFGF) (All products are from Gibco Invitrogen corporation products, San Diego, CA, USA).

LIF medium—10, basal medium-1 supplemented with 3000 U/ml LIF (PeproTech) and 10 ng/ml basic fibroblast growth factor (bFGF) (All products are from Gibco Invitrogen corporation products, San Diego, CA, USA).

Wnt3a medium, basal medium-1 supplemented with 10 ng/ml Wnt3a (R&D Systems) and 100 ng/ml basic fibroblast growth factor (bFGF) (All products from Gibco Invitrogen corporation products, San Diego, CA, USA). It should be noted that bFGF can be used at a concentration range between 4-100 ng/ml).

Protease inhibitor and IL6RIL6 chimera medium: basal medium-I supplemented with a protease inhibitor such as phenylmethylsulfonyl fluoride (PMSF) at a concentration of 100 μM, or Tosyl-L-lysyl-chloromethane hydrochloride (TLCK) at a concentration of 50 μM, and the IL6RIL6 chimera at a concentration of 100 μg/ml.

Protease inhibitor and a GP130 agonist medium: basal medium-1 supplemented with a protease inhibitor such as phenylmethylsulfonyl fluoride (PMSF) at a concentration of 100 μM, or Tosyl-L-lysyl-chloromethane hydrochloride (TLCK) at a concentration of 50 μM, and a GP130 agonist. The GP130 agonist can be interleukin 6 (IL6) (e.g., at a concentration of 100 ng/ml), interleukin 11 (“IL11” or “IL-11”, which is interchangeably used herein) (e.g., at a concentration of 1 ng/ml), LIF (e.g., at a concentration of 3000 U/ml), or Ciliary neurotrophic factor (CNTF) (e.g., at a concentration of 1 ng/ml).

Wnt3a medium: basal medium-1 supplemented with 10 ng/ml Wnt3a (R&D Systems) and 100 ng/ml basic fibroblast growth factor (bFGF) (All products from Gibco Invitrogen corporation products, San Diego, CA, USA). It should be noted that bFGF can be used at a concentration range between 4-100 ng/ml).

Wnt3a+chimera medium: basal medium-1 supplemented with 10 ng/ml Wnt3a (R&D Biosystem) and 100 μg/ml IL6RIL6 chimera.

yF10: basal medium-1 supplemented with 10 ng/ml basic fibroblast growth factor (bFGF).

yF100: basal medium-1 supplemented with 100 ng/ml basic fibroblast growth factor (bFGF).

BFGF (10) and TGFβ1: basal medium-1 supplemented with transforming growth factor beta-1 (TGFβ1) 0.12 ng/ml and 10 ng/ml basic fibroblast growth factor (bFGF).

BFGF (100) and TGFβ1: basal medium-1 supplemented with TGFβ1 0.12 ng/ml and 100 ng/ml basic fibroblast growth factor (bFGF).

CNTF and IL11 medium: DMEM/F12 91.8% v/v, KoSR 5% v/v, NEAA Non Essential Amino Acid) 1% (v/v), CNTF 1 ng/ml, IL-11 1 ng/ml, beta mercaptoethanol 0.1 mM, L-Gluthmine 1 mM, bFGF 20 ng/ml, Penicillin 50 U/ml, Streptomycin 0.05 mg/ml.

PMSF medium: DMEM/F12 92.8% v/v, KoSR 5% v/v, NEAA (Non Essential Amino Acid) 1% (v/v), beta mercaptoethanol 0.1 mM, L-Gluthmine 1 mM, PMSF at a concentration in the range of 70-130 μM (exact concentration is described in each experiment or Figure).

It is noted that the PMSF medium does not include bFGF.

Defined “IT1” medium: DMEM/F12 (94.7%), insulin 0.43 μM (Sigma Catalogue No. 19287), Transferrin 0.0172 μM (Holo Transferrin Sigma T0665), lipid mixture 1% volume/volume (Gibco, Catalogue No. 11905-031), bovine serum albumin 0.5% v/v (Sigma Catalogue No. A9418), bFGF (50 ng/ml), IL6RIL6 chimera 100 μg/ml (R&D Cat: 8954-SR-025), ascorbic acid 500 μg/ml (RND-SOL-041), L-glutamine 4 mM, Penicillin 50 U/ml, Streptomycin 0.05 mg/ml.

It is noted that the “IT1” medium does not include any added NEAA.

Defined “IT2” medium: DMEM/FF12 (94.7%), insulin 1.57 μM (Sigma Catalogue No. I9287), Transferrin 0.055 μM (Holo Transferrin Sigma T0665), lipid mixture 1% volume/volume (Gibco, Catalogue No. 11905-031), bovine serum albumin 0.5% v/v (Sigma Catalogue No. A9418), bFGF (50 ng/ml), IL6RIL6 chimera 100 μg/ml (R&D Cat: 8954-SR-025), ascorbic acid 500 μg/ml (RND-SOL-041), L-glutamine 4 mM, Penicillin 50 U/ml, Streptomycin 0.05 mg/ml.

It is noted that the “IT2” medium does not include any added NEAA.

### EB Formation:

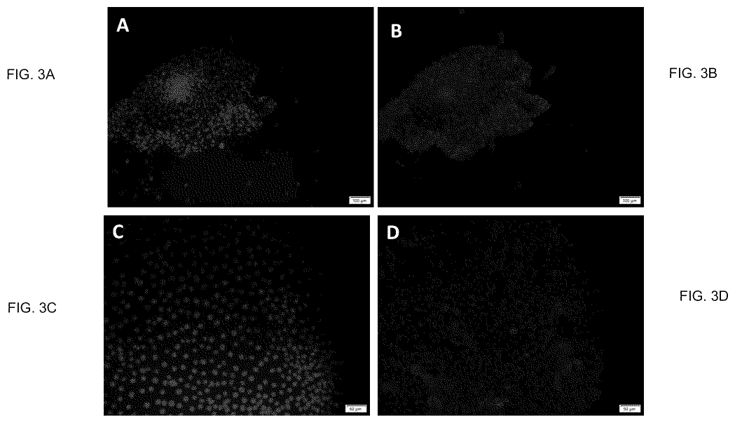

For the formation of embryoid bodies (EBs) two of four confluent wells in a four-well plate were used. The cells were cultured in the presence of the medium X and were left without splitting for 14 days to reach a confluent culture, and EBs were spontaneously formed. Some remained attached to culture surface and some floating EBs (FIGS. 3A-D). EBs were grown using medium X.

### Cell Culture:

Adherent culture: Medium was changed every day except for one day per week. Cells were split every 5-10 days using Collagenase type-4. The cells were frozen in liquid nitrogen using a freezing solution consisting of 10% Dimethyl sulfoxide (DMSO) (Sigma, St Louis, MO, USA), 10% FBS (Hyclone, Utah, USA) and 80% DMEM\12.

### Suspension Culture:

1. Adaptation to suspension, the cells are split using an enzyme such as TrypLEx (Gibco-Invitrogen Corporation, Grand Island NY, USA), Trypsin EDTA (BI), accutase or collagenase type IV (Worthington), and transferred to Petri dishes. Alternatively, cells could be split by mechanical dissociation of cell clumps by pipetting up and down the cell clumps using 200-1000 μL Gilson tips.
2. Cells are cultured for 3-5 passages, split every 5-10 days (as described in 1 above).
3. Cells could be transferred to spinner flasks (75 rpm (Revolutions Per Minute). No splitting is needed.
4. Cells could be transferred to Bioreactors.

### Immunostaining:

Cells were fixed and exposed to the primary antibodies at room temperature. Then the cells were incubated with secondary antibodies. Table 1 below summarized the reaction condition and antibodies.

TABLE 1
Table 1: Immunostaining conditions

| | |
|---|---|
| Fixation: PFA 4% | Fixation: Methanol |
| Permeabilization buffer: 0.5% Triton in | Permeabilization buffer: NA |
| PBS | Blocking Buffer: 5% Host serum + 0.2% |
| Blocking Buffer: 5% Host serum + 0.2% | Tween in PBS |
| Tween in PBS | Alpha 1 Fetoprotein (AFP) (mouse) 1:200 |
| β III Tubulin (TUBB3) (rabbit) 1:100 | α-Actinin (ACTN1) (rabbit) 1:100 |
| EOMES (mouse) 1:100 | TRA1-60-R 1:400 |
| Oct3/4 (POU5F1)1:100 | TRA1-81 1:100 |
| Nanog 1:200 | |
| Fixation: PFA 4% | Fixation: Methanol |
| Permeabilization buffer: 0.5% Triton in | Permeabilization buffer: NA |
| PBS | Blocking Buffer: 5% Host serum + 0.2% |
| Blocking Buffer: 5% Host serum + 0.2% | Tween in PBS |
| Tween in PBS | Nestin (rabbit) 1:100 |
| | α-Actinin (ACTN1) (mouse) 1:100 |

Spontaneous differentiation toward adipocytes: Cells cultured with either medium X or with 15% ko-SR and the IL6RIL6 Chimera medium (including 50 ng/ml bFGF) on MEFs feeder layer were spontaneously differentiated to adipocyte as background differentiation or when left without passaging for more than 14 days.

### Oil Red Staining:

Cells were fixed with paraformaldehyde (PFA) 4% for 20 minutes (min) at room temperature (RT). After washing the PFA with PBS, the cells are incubated with Oil Red O solution (Sigma) for 10 min at RT. The culture is washed with water and visualized by phase contrast microscope.

### Example 1

### Low Concentrations of Serum Replacement can Support the Undifferentiated Growth of Mammalian Livestock Pluripotent Stem Cells

The present inventor has tested the ability of various medium formulations with low concentrations of serum replacement (e.g., between 1-10% of KoSR) to support the undifferentiated and pluripotent state of mammalian livestock pluripotent stem cells

### Experimental Results

Several medium formulations based on different growth factors combinations and low concentration of serum replacer were tested for the ability to support the growth of mammalian livestock pluripotent stem cells (iPSCs, extended blastocyst ESCs, or ESCs) under various culture systems, such as on feeder cell layers (e.g., MEFs), or feeder-layer free culture systems such as in suspension and or synthetic matrixes (feeder-free adherent cultures). The tested medium formulations were found suitable to support undifferentiated PSC proliferation and maintenance of PSCs characteristics.

### Culturing on Feeder Cell Layers

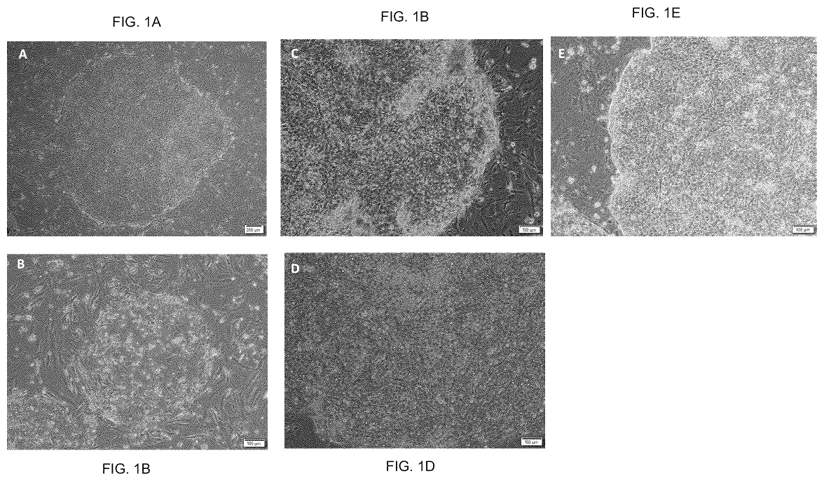

Bovine iPSCs maintain an undifferentiated state when cultured on feeder cells for at least 5 passages in a medium supplemented with 5% KoSR (KNOCKOUT™ serum replacement)—As shown in FIGS. 1A-B, iBVN 1.14 p7+23 cells, which were cultured on MEFs for 5 passages with YF10 supplemented with 5% KoSR, maintain a morphology of undifferentiated pluripotent stem cells. Similarly, iBVN1.14 P7+29 cells which were cultured on MEFs for 6 passages with the Wnt3a+IL6RIL6 Chimera (with 50 ng/ml bFGF) medium supplemented with 5% KoSR, maintain a morphology of undifferentiated pluripotent stem cells (FIG. 1E).

Bovine ESCs maintain an undifferentiated state when cultured on feeder cells for at least 5 passages in a medium supplemented with 5% KoSR (KNOCKOUT™ serum replacement)—The bovine ESCs (BVN3 P5 and BVN4 P8) which were cultured on MEFs with the IL6RIL6 Chimera medium (with 50 ng/ml bFGF) supplemented with 5% KoSR maintained a morphology of undifferentiated pluripotent stem cells (FIGS. 1C-D).

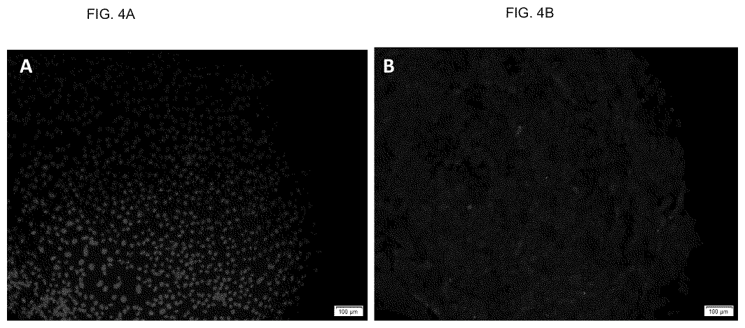

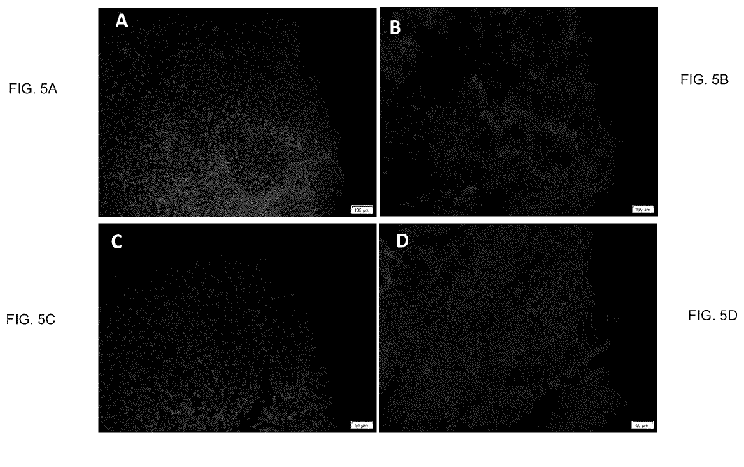

Bovine iPSCs maintain an undifferentiated and pluripotent state when cultured on feeder cells for at least 5 passages in a medium supplemented with 5% KoSR (KNOCKOUT™ serum replacement)—iBVN 1.4 p7+30 which were cultured on MEFs for 17 passages with the YF10 medium supplemented with 5% KoSR exhibit positive staining of the pluripotent stem cells marker TRA-1-81 (FIGS. 4A-B, images were captured after 11 passages of culturing with the YF10 medium supplemented with 5% KoSR).

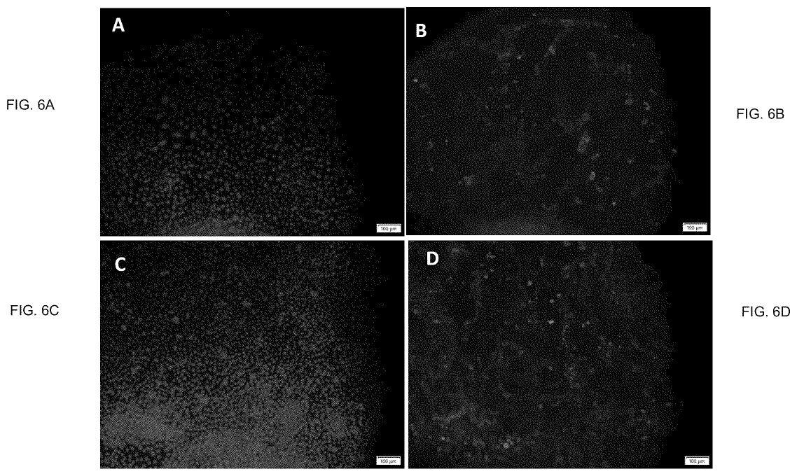

Similarly, iBVN 1.4 p7+30 which were cultured on MEFs for 13 passages with the IL6RIL6 Chimera medium (with 50 ng/ml bFGF) supplemented with 5% KoSR exhibit positive staining of the pluripotent stem cells markers TRA-1-60 and TRA-1-81 (FIGS. 5A-D, images were captured after 11 passages of culturing with the IL6RIL6 Chimera medium (with 50 ng/ml bFGF) supplemented with 5% KoSR).

In addition, iBVN 1.14 p7+29 cells which were cultured on MEFs for 13 passages with the IL6RIL6 Chimera medium (with 50 ng/ml bFGF) supplemented with 5% KoSR exhibit positive staining for the TRA-1-60 and TRA-1-81 pluripotent stem cells markers (FIGS. 6A-D, images were captured after 10 passages of culturing with the IL6RIL6 Chimera medium (with 50 ng/ml bFGF) supplemented with 5% KoSR).

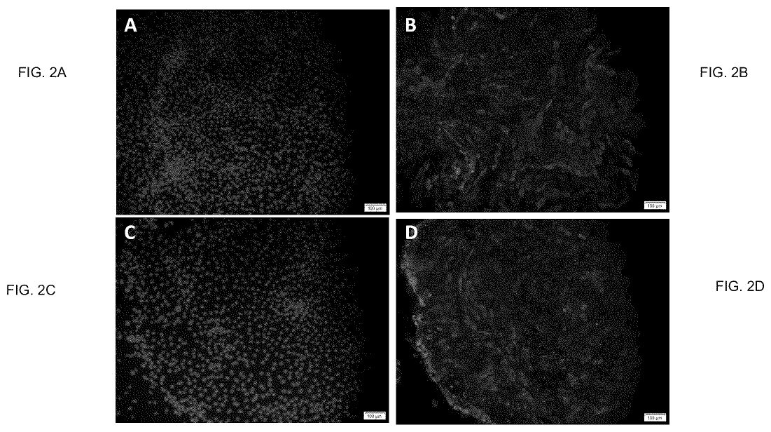

Bovine iPSCs maintain an undifferentiated and pluripotent state when cultured on feeder cells for at least 5 passages in a medium supplemented with 5-10% KoSR (KNOCKOUT™ serum replacement)—iBVN 1.4 p7+27 cells which were cultured on MEFs for 8 passages with the YF10 medium supplemented with 10% KoSR exhibit positive staining of the pluripotent stem cells markers TRA-1-60 and TRA-1-81 (FIGS. 2A-D).

Similarly, iBVN 1.4 p7+30 cells which were cultured on MEFs for 11 passages with the YF10 medium supplemented with 5% KoSR medium exhibit positive staining of the pluripotent stem cells markers Nanog and TRA-1-60 (FIGS. 3A-D).

Bovine iPSCs maintain an undifferentiated state when cultured on feeder cells for at least 5 passages in a medium supplemented with 1-2.5% KoSR (KNOCKOUT™ serum replacement)—iBVN1.4 p7+43 cells which were cultured on MEFs for 13 passages with the IL6RIL6 Chimera medium (with 50 ng/ml bFGF) supplemented with 2.5% KoSR exhibit a morphology of undifferentiated state (FIG. 13A, images were captured after 7 passages of culturing with the IL6RIL6 Chimera medium (with 50 ng/ml bFGF) supplemented with 2.5% KoSR). Similarly, when the same cells were cultured on MEFs for 13 passages with the IL6RIL6 Chimera medium (with 50 ng/ml bFGF) supplemented with 1% KoSR the cell colonies remained in the undifferentiated state (FIG. 13B, images were captured after 7 passages of culturing with the IL6RIL6 Chimera medium (with 50 ng/ml bFGF) supplemented with 1% KoSR).

### Culturing in Suspension

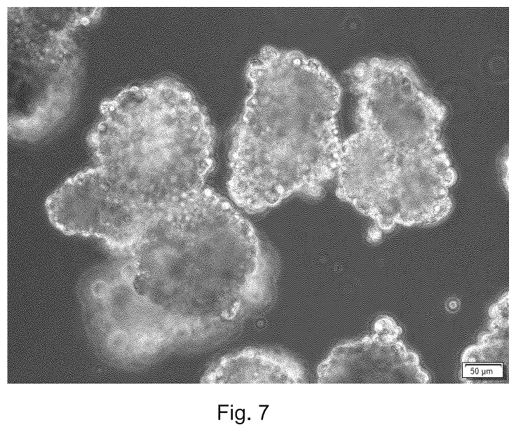

Bovine iPSCs maintain an undifferentiated state when cultured in a suspension culture for at least 5 passages in a medium supplemented with 10% KoSR (KNOCKOUT™ serum replacement)—iBVN1.4 p7+27 cells which were cultured in suspension for one month with the IL6RIL6 Chimera medium (with 50 ng/ml bFGF) supplemented with 10% KoSR maintain a morphology of undifferentiated Bovine iPSC cells (FIG. 7).

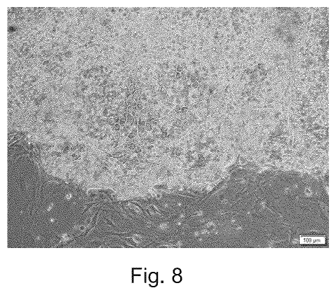

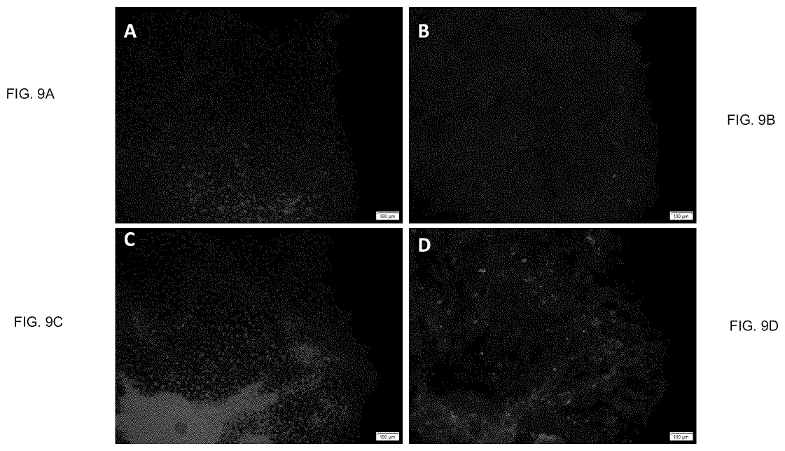

Bovine iPSC maintain an undifferentiated state when cultured in suspension for at least 5 passages in a medium supplemented with 5% KoSR (KNOCKOUT™ serum replacement)—iBVN1.4 p7+17 cells which were cultured in suspension for one month with the IL6RIL6 Chimera medium (with 50 ng/ml bFGF) supplemented with 5% KoSR, and were then re-plated on MEFs maintain the morphology of undifferentiated Bovine iPSC cells (FIG. 8).

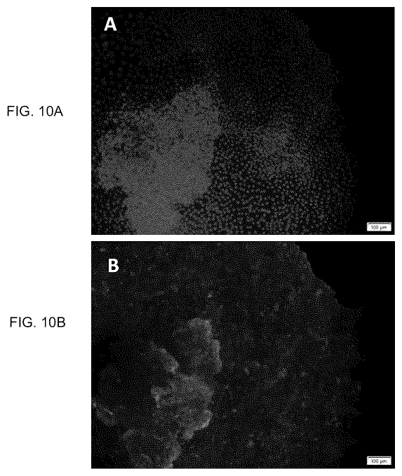

Bovine iPSCs maintain an undifferentiated and pluripotent state when cultured in suspension for at least 5 passages in a medium supplemented with 5% KoSR (KNOCKOUT™ serum replacement)—iBVN 1.14 p7+30 cells which were cultured in suspension for 1 month with the IL6RIL6 Chimera medium (with 50 ng/ml bFGF) supplemented with 5% KoSR exhibit positive staining of the pluripotent stem cells markers Nanog and TRA-1-60 (FIGS. 9A-D) as well as TRA-1-81 (FIGS. 10A-B).

### Example 2

### Effect of Increasing Concentrations of Serum Replacement on Background Differentiation of Mammalian Livestock Pluripotent Stem Cells

The present inventor has tested the ability of various medium formulations with increasing concentrations of serum replacement (e.g., between 10-15% of KoSR) to support the undifferentiated and pluripotent state of mammalian livestock pluripotent stem cells

### Experimental Results

### Culturing on Feeder Cell Layers

Bovine iPSCs exhibit some degree of background differentiation to adipocyte cells when cultured on feeder cells for at least 5 passages in a medium supplemented with 10% KoSR (KNOCKOUT™ serum replacement)—iBVN1.4 p7+42 cells which were cultured on MEFs in the presence of the IL6RIL6 Chimera medium (with 50 ng/ml bFGF) supplemented with 10% KoSR show some degree of background differentiation. As shown in FIGS. 12A-D, some of the colonies differentiated, mainly to fat cells (FIGS. 12A and 12C), and in other colonies areas with fat cells (marked with white arrow) could be noted (FIGS. 12B and 12D). Differentiation into adipocytes was confirmed with oil red staining (FIGS. 12C and 12D).

Bovine iPSCs exhibit some degree of background differentiation to adipocyte cells when cultured on feeder cells for at least 5 passages in a medium supplemented with 15% KoSR (KNOCKOUT™ serum replacement)—iBVN1.4 p7+42 cells which were cultured on MEFs for 3 passages with the IL6RIL6 Chimera medium (with 50 ng/ml bFGF) supplemented with 15% KoSR show some degree of differentiation. As shown in FIGS. 11A-D some of the colonies differentiated, mainly to fat cells (FIGS. 11A and 11C), and in other colonies areas with fat cells (marked with white arrow) could be noted (FIGS. 11B and 111D). The differentiation into adipocytes was confirmed with the oil red staining (FIGS. 11C and 111D).

### Example 3

### Derivation of a Bovine Embryonic Stem Cell in a Medium Supplemented with 5% Serum Replacement

### Experimental Results

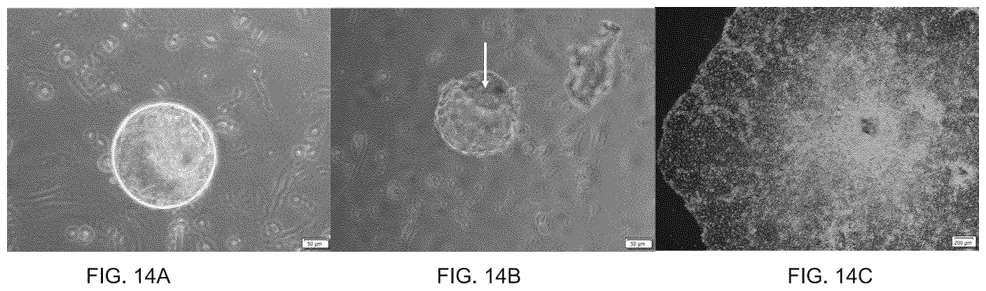

FIGS. 14A-C depict the derivation of BVN3 cell line in the IL6RIL6 chimera medium (with 50 ng/ml bFGF) supplemented with 5% KoSR. ESC line BVN3 was derived using a whole embryo approach. The results show that bovine embryonic stem cells can be derived in a medium supplemented with low concentrations of serum replacement such as 5% KoSR.

### Example 4

### Comparative Data for Colony Diameter of Bovine Pluripotent Stem Cells Cultured in Various Low Concentrations of Serum Replacement

### Experimental Results

The present inventor has compared the effect of the concentration of serum replacement on colony cell growth, by measuring the diameter of bovine iPSC colonies (of the iBVN1.4 cell line at passage 42 and 43) at three days post splitting of the colonies (passaging). The iBVN1.4 cells were cultured on MEFs with the IL6RIL6 chimera medium (with 50 ng/ml bFGF) supplemented with different concentrations of KoSR. As shown in FIG. 15, at concentrations of 1% or 2.5% of KoSR the average diameter of colonies is smaller as compared to the diameter of colonies grown in the same medium supplemented with 5% KoSR or with higher concentrations of 7.5%, 10% or 15% KoSR. No significant difference was found between the diameters of colonies when grown in a medium supplemented with 5-15% KoSR.

### Analysis and Summary

Bovine pluripotent stem cells were cultured in the tested medium formulations for at least 27 passages and maintained their PSC features, including undifferentiated proliferation, cells and colony morphology, and pluripotency.

In addition, bovine PSCs cultured in the tested culture medium strongly expressed specific pluripotency markers such as Nanog, OCT4 (Data not shown), TRA-1-60 and TRA-1-81.

As shown in FIG. 15, the colony diameter of PSCs cultured in as low as 1-2.5% KoSR is smaller than that of cells cultured with 5% KoSR or with higher concentrations of 7.5%, 10% or 15% KoSR, indicating a somewhat slower growth rate of colonies during the first 1-7 passages in the presence of 1% or 2.5% KoSR. On the other hand, it should be noted that at concentrations of 1-2.5% KoSR there is no significant background differentiation of the PPSCs (described in Example 1 above and in FIGS. 13A-B, less than 3% background differentiation) and at a concentration of 5% KoSR there is about 5% background differentiation to adipocyte cells. In contrast, at a concentration of 10% KoSR there is about 10% background differentiation to adipocyte cells (FIGS. 12A-D); and at a concentration of 15% KoSR there is about 15-20% background differentiation to adipocyte cells (FIGS. 11A-D), thus increasing the concentration of KoSR from 5% to 10% or 15% results in increasing of background differentiation.

The developmental potential of the cells after prolonged culture while using the tested new formulations was examined in vitro by the formation of embryoid bodies (EBs) (Data not shown). When cultured in suspension, e.g., after 10 passages, in the tested medium PSCs formed EBs containing representative cells for the three embryonic germ layers. Additionally, oil red staining demonstrated the ability of the cells to differentiate into fat cells.

Thus, the new tested medium formulations were found suitable for prolonged culture of PSCs while maintaining PSC characteristics.

### Example 5

### Culture Media with Low Concentrations of Serum Replacement and Supplemented with CNTF and IL11 Maintain Livestock Pluripotent Stem Cells in an Undifferentiated State

The CNTF and IL11 culture medium which comprises 5% % of KoSR, 1 ng/ml CNTF and 1 ng/ml IL11 was used to culture bovine and porcine pluripotent stem cells (iPSCs) on two-dimensional cultures (on MEFs feeder cells) or three-dimensional suspension cultures.

### Experimental Results

### Two-Dimensional Culture Systems

As shown in FIGS. 16A-B and 23A-D, bovine iPSCs which are cultured on MEFs with the CNTF and IL11 medium maintain their undifferentiated and pluripotent state for at least 3 or 5 passages, showing positive expression of the pluripotency markers TRA1-60, Nanog, OCT4, and TRA1-81.

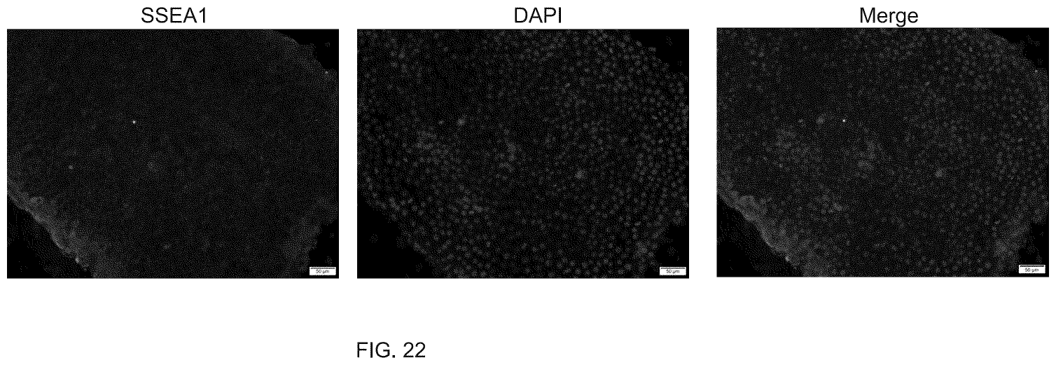

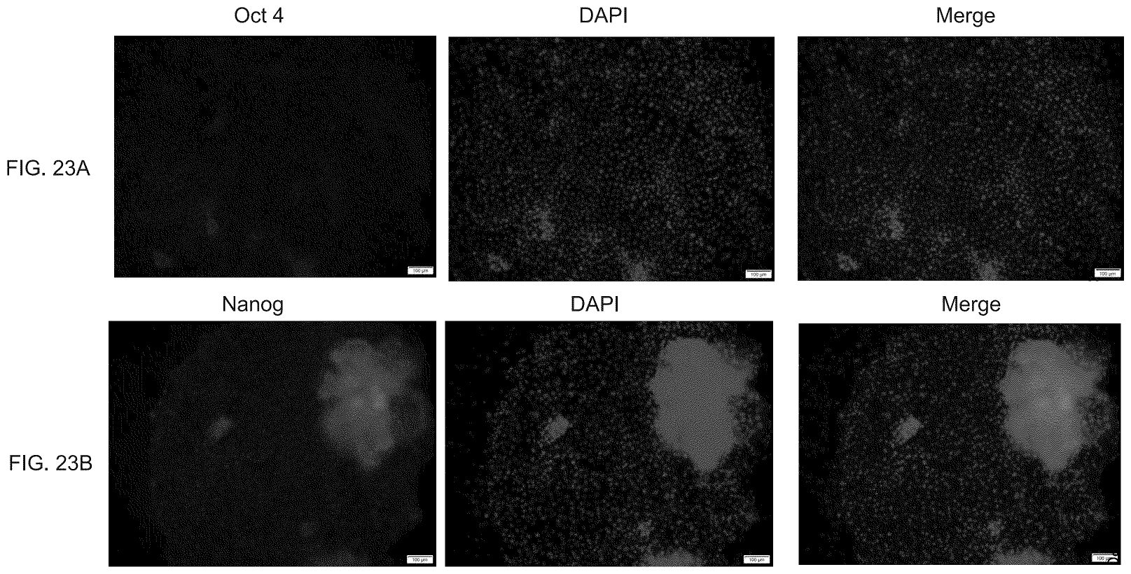

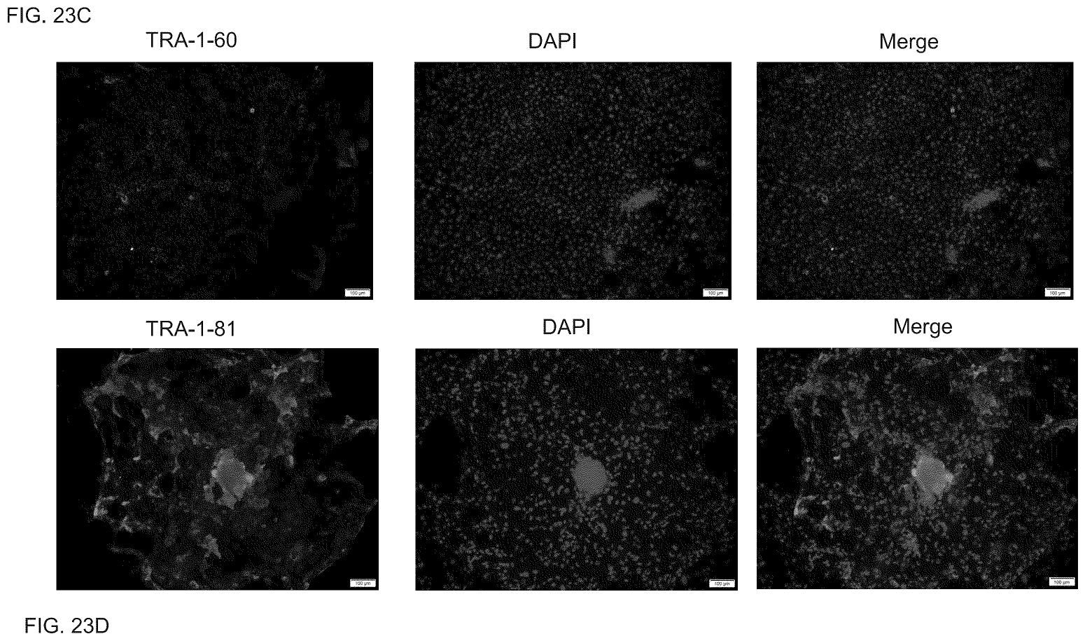

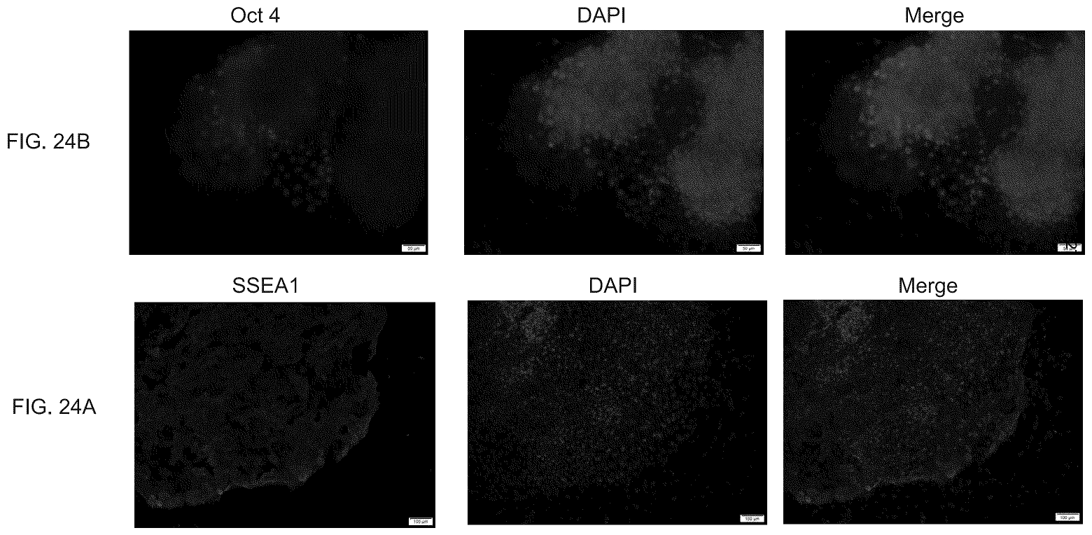

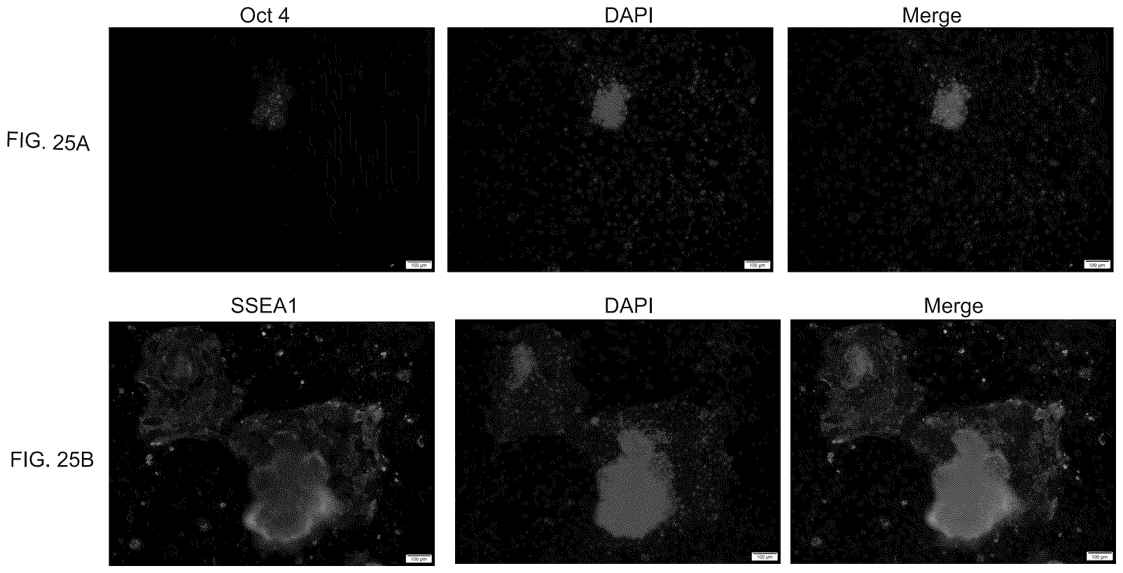

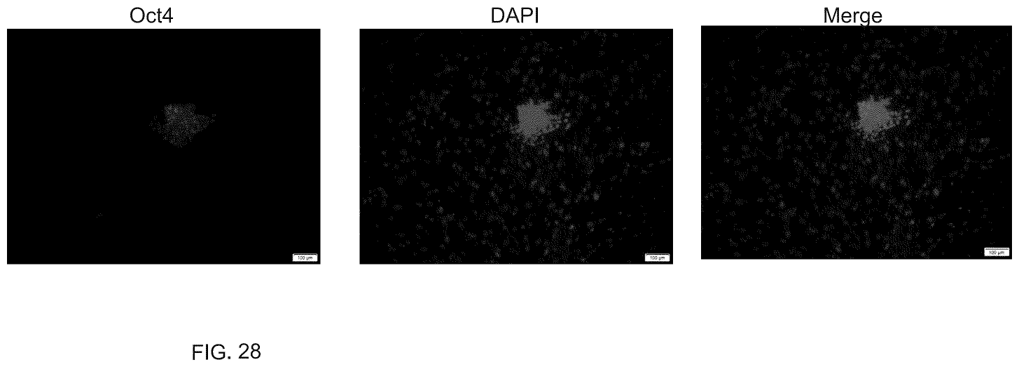

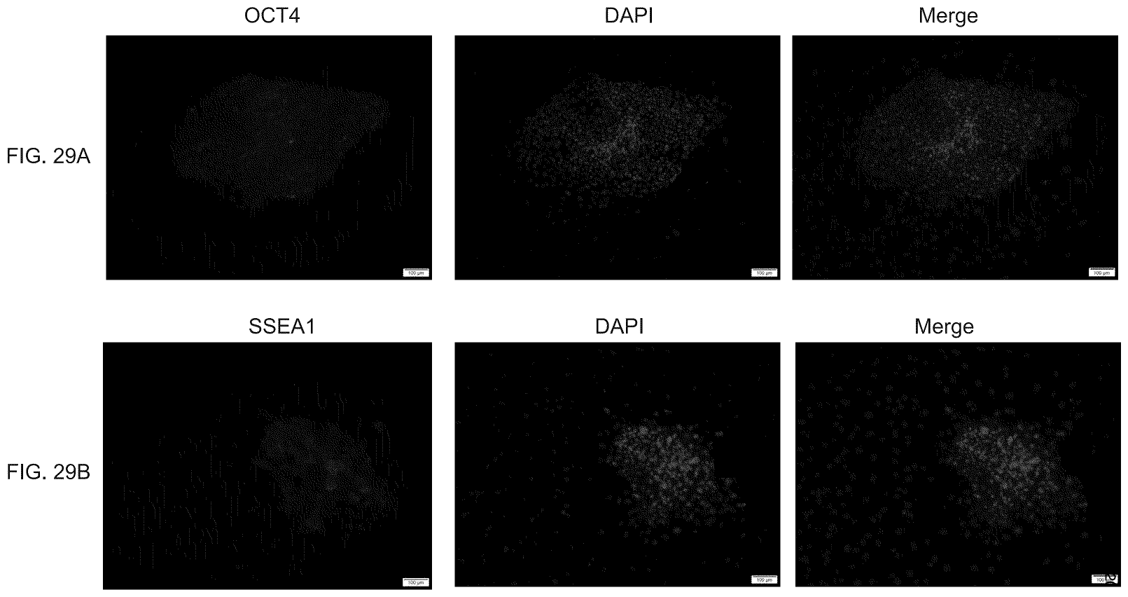

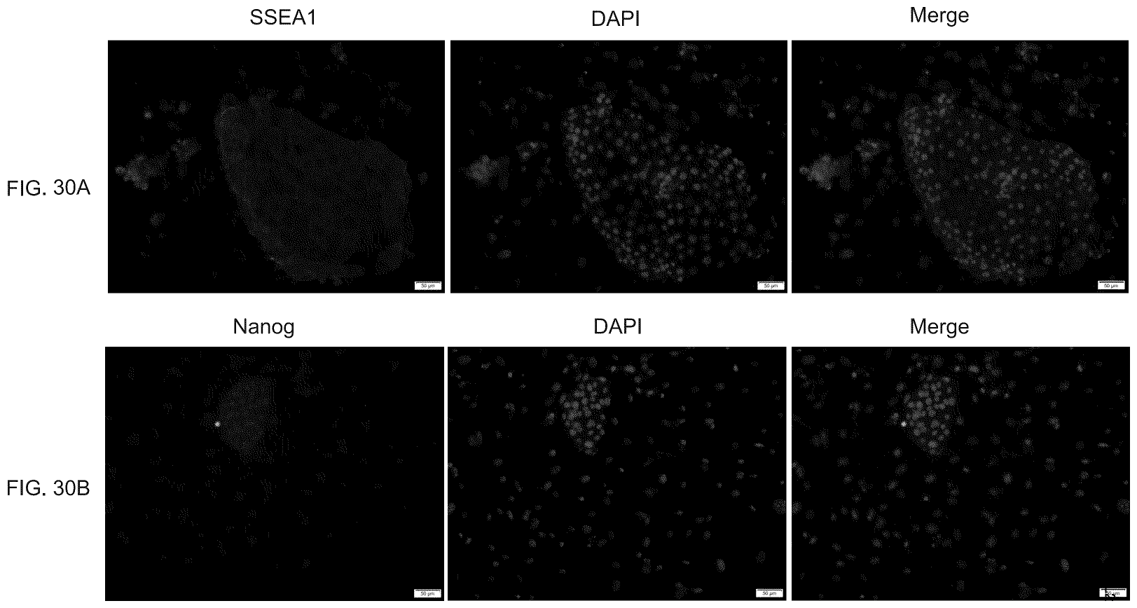

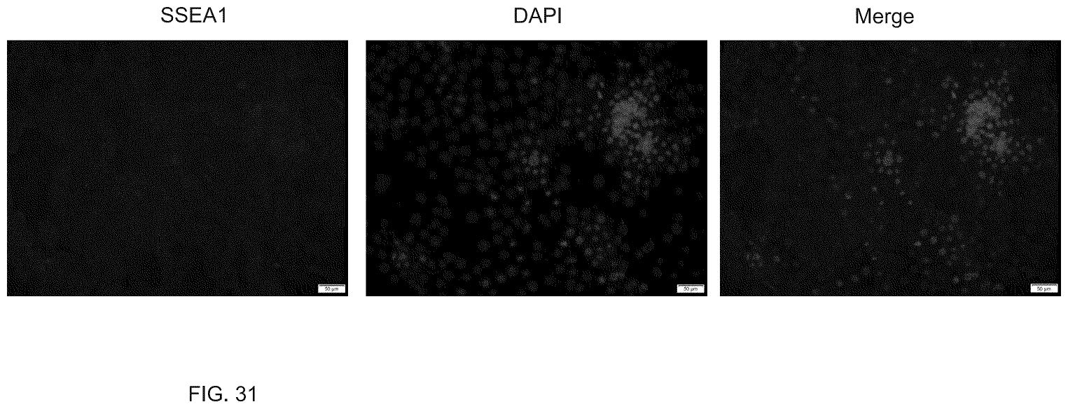

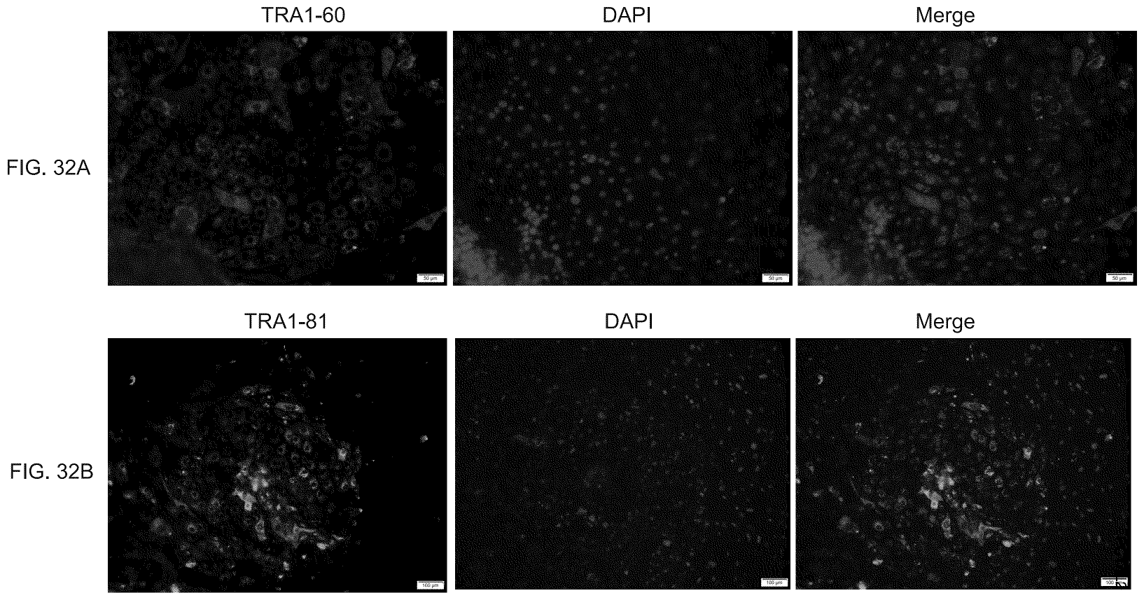

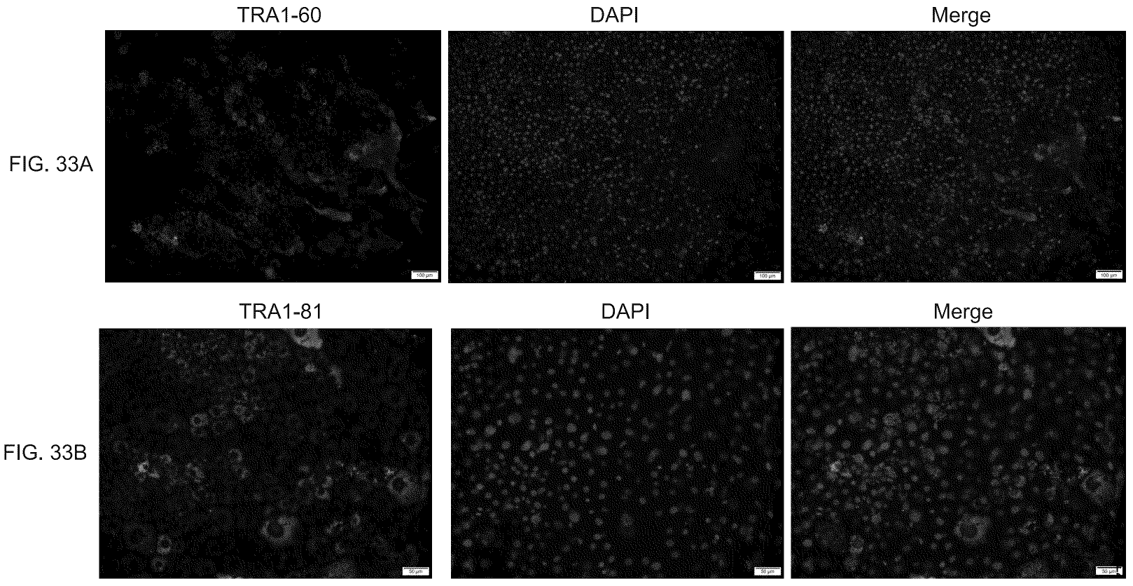

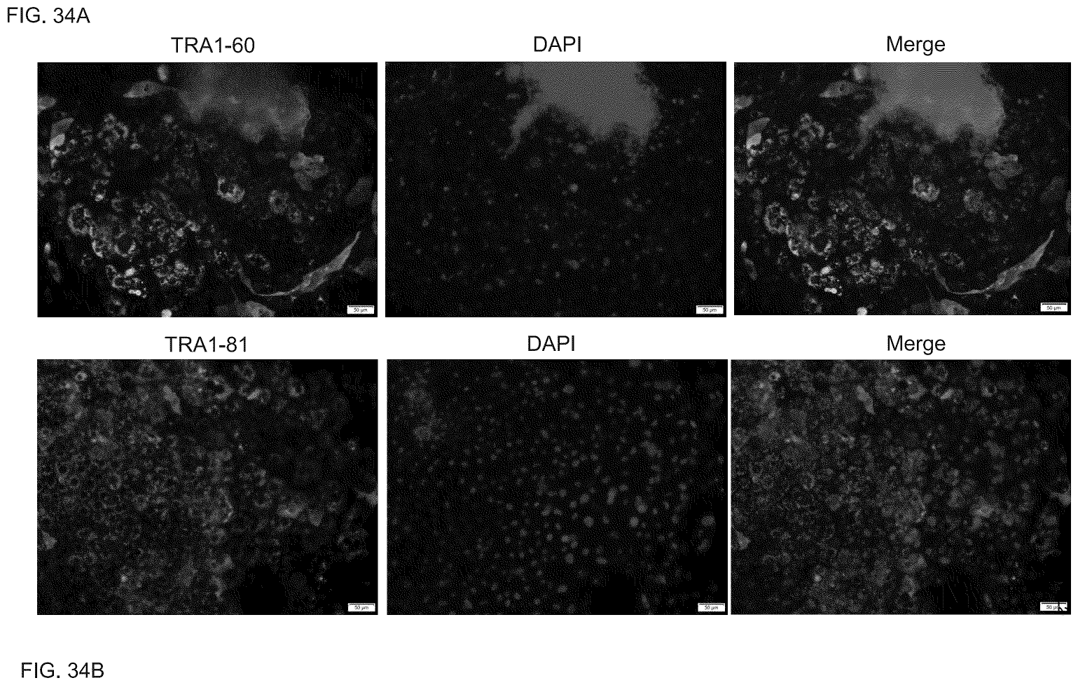

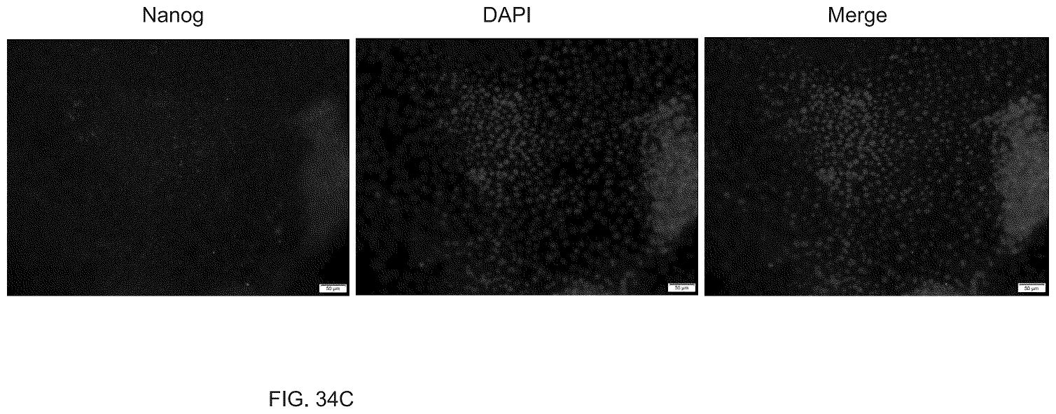

Similarly, as shown in FIGS. 22 and 28, porcine iPSCs which are cultured on MEFs with the CNTF and IL11 medium maintain their undifferentiated and pluripotent state for at least 3 or 5 passages, showing positive expression of the pluripotency markers SSEA1 and OCT4.

### Three-Dimensional Culture Systems

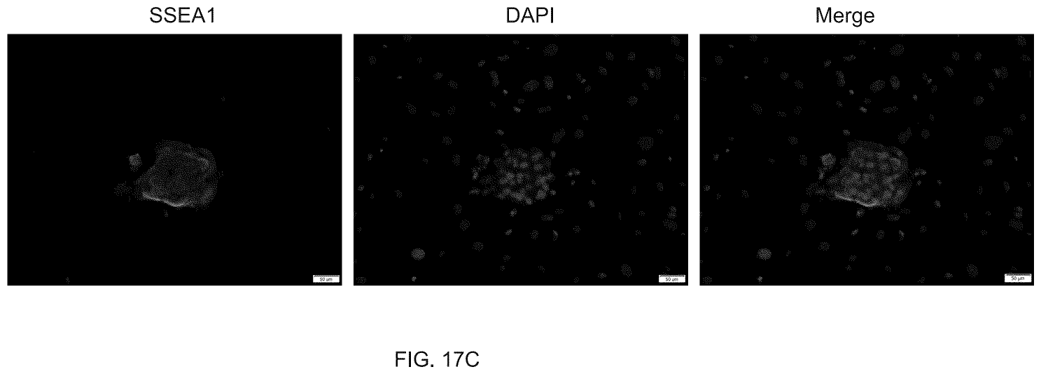

As shown in FIGS. 17A-C and 24A-B, porcine iPSCs which are cultured in a 3-D suspension culture with the CNTF and IL11 medium maintain their undifferentiated and pluripotent state for at least 3 or 5 passages, showing positive expression of the pluripotency markers Nanog, OCT4, and SSEA1.

### Example 6

### Culture Media with Low Concentrations of Serum Replacement and Supplemented with a Protease Inhibitor Maintain Livestock Pluripotent Stem Cells in an Undifferentiated State

PMSF culture media which comprises 5% of KoSR, and 70, 100 or 130 μM PMSF were used to culture bovine and porcine pluripotent stem cells (iPSCs) on two-dimensional cultures (on MEFs feeder cells) or three-dimensional suspension cultures.

### Experimental Results

### Two-Dimensional Culture Systems

As shown in FIGS. 18A-B, 19, 21A-B, 29A-B, and 34A-C, bovine or porcine iPSCs which are cultured in a suspension culture (3D) with the PMSF medium maintain their undifferentiated and pluripotent state for at least 3 or 5 passages, showing positive expression of the pluripotency markers TRA1-60, Nanog, OCT4, SSEA1, and TRA1-81.

### Three-Dimensional Culture Systems

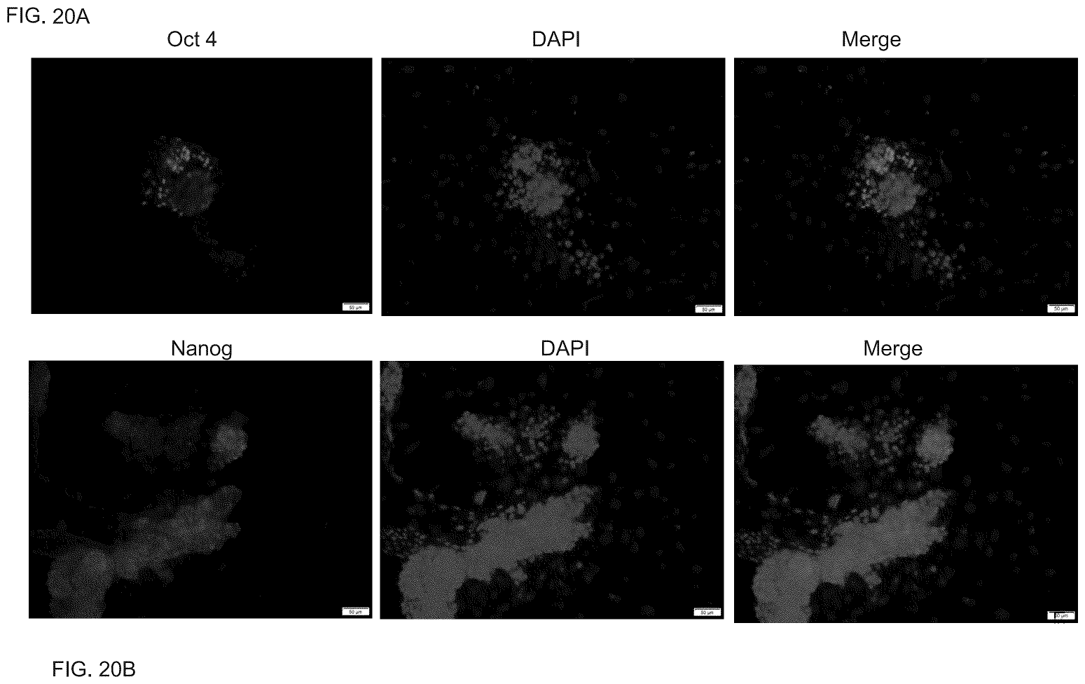

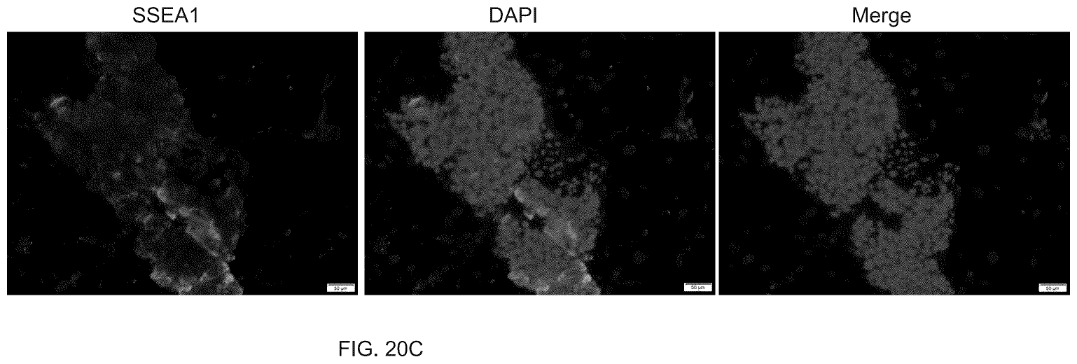

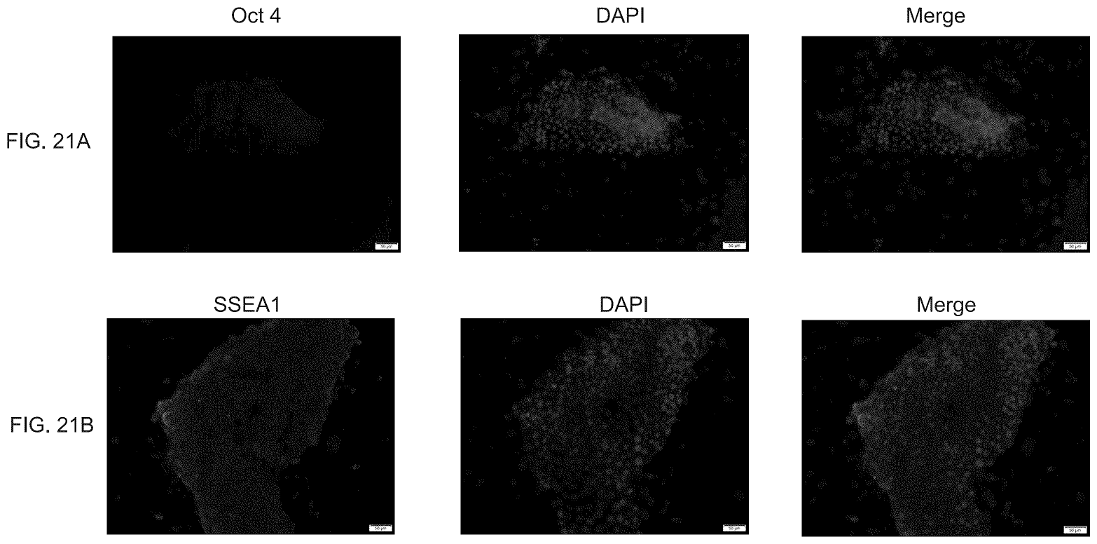

As shown in FIGS. 20A-C and 25A-B, porcine iPSCs which are cultured in a 3D suspension culture with the PMSF media maintain their undifferentiated and pluripotent state for at least 3 or 5 passages, showing positive expression of the pluripotency markers Nanog, OCT4, and SSEA1.

### Example 7

### Defined Culture Media with Insulin and Transferrin Maintain Livestock Pluripotent Stem Cells in an Undifferentiated State

The present inventors have tested the ability of chemically defined culture medium to maintain the undifferentiated growth of mammalian livestock pluripotent stem cells. The chemically defined culture media comprise insulin and transferrin, along with a lipid mixture, BSA, and supplemented with differentiation inhibitory factors (bFGF, IL6RIL6 chimera and ascorbic acid), however, they do not comprise selenium. The first chemically-defined culture medium, termed “IT1”, includes 0.43 μM insulin and 0.0172 μM transferrin. The second chemically-defined culture medium, termed “IT2”, includes 1.57 μM insulin and 0.055 μM transferrin.

### Experimental Results

### Two-Dimensional Culture Systems

As shown in FIGS. 26A-B, 27A-B, 30A-B, 31, 32A-B and 33A-B bovine and porcine iPSCs which are cultured on MEFs with the defined culture media (“IT1” or “IT2”) maintain their undifferentiated and pluripotent state for at least 3 or 5 passages, showing positive expression of the pluripotency markers TRA1-60, Nanog, SSEA1, and TRA1-81.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A defined serum-free culture medium comprising a basal medium, serum replacement and an effective concentration of at least one differentiation inhibiting agent, wherein the defined culture medium is capable of maintaining mammalian livestock pluripotent stem cells in an undifferentiated state for at least 5 passages in culture, wherein said basal medium is selected suitable for maintaining pluripotent stem cells in an undifferentiated state, wherein said serum replacement comprises insulin and transferrin, and wherein said serum replacement comprises selenium in a concentration that does not exceed 2.23×10⁻⁴ gram per liter of said defined serum-free culture medium.

2. The defined serum-free culture medium of claim 1, wherein said insulin is provided at a concentration in a range of 0.34×10⁻³ mM to 1.88×10⁻³ mM, and wherein said transferrin is provided at a concentration in a range of 0.137×10⁻⁴ mM to 0.66×10⁻⁴ mM.

3. (canceled)

4. The defined culture medium of claim 1, with the proviso that said basal medium is not RPMI1640.

5. The defined culture medium of claim 1, wherein said basal medium is selected from the group consisting of KO-DMEM, DMEM/F12 and DMEM.

6. The defined culture medium of claim 1, wherein said basal medium is selected from the group consisting of KO-DMEM and DMEM/F12.

7. The defined culture medium of claim 1, wherein said basal medium is provided at a concentration in a range of 94-96%.

8. The defined culture medium of claim 1, wherein the culture medium is devoid of a cryoprotectant.

9. (canceled)

10. The defined culture medium of claim 1, wherein said medium does not comprise selenium.

11. The defined culture medium of claim 1, further comprising a lipid mixture at a concentration range of 0.5-1.2% (v/v).

12. The defined culture medium of claim 1, wherein said serum replacement further comprises ascorbic acid at a concentration in a range of 125-170 mM.

13. The defined culture medium of claim 1, wherein said serum replacement further comprises bovine serum albumin at a concentration in a range of 0.4% to 0.7% volume/volume (v/v).

14. The defined culture medium of claim 1, wherein said serum replacement is knockout (KO)-serum replacement provided at a concentration in a range of 1-10% volume/volume (v/v).

15. The defined culture medium of claim 1, wherein said at least one differentiation inhibiting agent is a growth factor, a cytokine, a small molecule, or a combination thereof, wherein said effective concentration of said at least one differentiation inhibiting agent is capable of maintaining said mammalian livestock pluripotent stem cells in an undifferentiated states for at least 5 passages in culture, and optionally wherein any one of:
a. said growth factor is basic fibroblast growth factor (bFGF), and optionally wherein said effective concentration of said bFGF is in a range of 4-110 ng/ml, said effective concentration of said bFGF is about 50 ng/ml, or both;
b. said at least one differentiation inhibiting agent is the IL6RIL6 chimera, and optionally wherein said effective concentration of said IL6RIL6 chimera is about 100 pg/ml;
c. said at least one differentiation inhibiting agent is a gp130 agonist, and optionally wherein said gp130 agonist is selected from the group consisting of leukemia inhibitory factor (LIF), interleukin-6 (IL6), interleukin-11 (IL11), and Ciliary neurotrophic factor (CNTF), said effective concentration of said IL11 is about 1 ng/ml, said effective concentration of said CNTF is about 1 ng/ml, or any combination thereof;
d. said at least one differentiation inhibiting agent comprises leukemia inhibitory factor (LIF) at a concentration of about 3000 U/ml and basic fibroblast growth factor (bFGF) at a concentration of about 50 ng/ml;
e. said at least one differentiation inhibiting agent comprises leukemia inhibitory factor (LIF) at a concentration of about 3000 U/ml and basic fibroblast growth factor (bFGF) at a concentration of about 10 ng/ml;
f. said at least one differentiation inhibiting agent comprises a Wnt3a polypeptide and basic fibroblast growth factor (bFGF), and optionally wherein said effective concentration of said Wnt3a polypeptide is about 10 ng/ml, said effective concentration of said bFGF is in a range of 4-100 ng/ml, or both;
g. said small molecule is a protease inhibitor selected from the group consisting of: phenylmethylsulfonyl fluoride (PMSF) and Tosyl-L-lysyl-chloromethane hydrochloride (TLCK), and optionally wherein said at least one differentiation inhibiting agent further comprises the IL6RIL6 chimera, said effective concentration of said IL6RIL6 chimera is in a range of 80-120 μg/ml, said effective concentration of said PMSF in a range of 70-130 μM, said effective concentration of said TLCK is in a range of 20-80 μM, or any combination thereof;
h. said at least one differentiation inhibiting agent comprises a gp130 agonist selected from the group consisting of leukemia inhibitory factor (LIF), interleukin-6 (IL6), interleukin-11 (IL11), and Ciliary neurotrophic factor (CNTF) and a protease inhibitor selected from the group consisting of phenylmethylsulfonyl fluoride (PMSF) and Tosyl-L-lysyl-chloromethane hydrochloride (TLCK);
i. said at least one differentiation inhibiting agent comprises a Wnt3a polypeptide and the IL6RIL6 chimera, and optionally wherein said effective concentration of said Wnt3a polypeptide is in a range of 5-20 ng/ml, and wherein said effective concentration of said IL6RIL6 chimera is in a range of 70-130 μg/ml;
j. said at least one differentiation inhibiting agent comprises any one of: basic fibroblast growth factor (bFGF), transforming growth factor beta, optionally wherein said TGFβ is selected from TGFβ1, and TGFβ3), and Activin; and
k. any combination of (a) to (j).

16.-38. (canceled)

39. The defined culture medium of claim 1, further comprises ascorbic acid, and optionally wherein said ascorbic acid is at a concentration range of 8-600 μg/ml or at a concentration range of 450-550 μg/ml.

40.-41. (canceled)

42. The defined culture medium of claim 1, wherein the culture medium comprises ascorbic acid at a concentration range of 450-550 μg/ml and basic fibroblast growth factor at a concentration of 40-60 ng/ml.

43. A cell culture comprising the defined culture medium of claim 1 and cells, and optionally wherein said cells are mammalian livestock pluripotent stem cells.

44. (canceled)

45. A method of maintaining mammalian livestock pluripotent stem cells in an undifferentiated state, comprising culturing the mammalian livestock pluripotent stem cells in the defined culture medium of claim 1, and optionally wherein any one of:
a. the method further comprising passaging the mammalian livestock pluripotent stem cells for at least one time, and optionally wherein said passaging is effected every 5-21 days during said culturing, passaging comprises splitting the mammalian livestock pluripotent stem cells in a 1 to 2, or a 2 to 3 ratio before further culturing said cells, or both;
b. said culturing is performed on feeder cell layers;
c. said culturing is performed on a feeder-free matrix;
d. said culturing is performed in a suspension culture devoid of substrate adherence; and
e. any combination of (a) to (d).

46.-51. (canceled)

52. A method of differentiating mammalian livestock pluripotent stem cells comprising:
(a) culturing the mammalian livestock pluripotent stem cells according to the method of claim 45, to thereby obtain an expanded population of mammalian livestock pluripotent stem cells in an undifferentiated state, and
(b) culturing said expanded population of mammalian livestock pluripotent stem cells in an undifferentiated state under conditions devoid of said differentiation inhibiting agent which allow differentiation of said mammalian livestock pluripotent stem cells,
thereby differentiating the mammalian livestock pluripotent stem cells,
and optionally wherein any one of:
a. said conditions comprise culturing said cells in a culture medium suitable for differentiating said mammalian livestock undifferentiated stem cells into muscle cells;
b. said conditions comprise culturing said cells in a culture medium suitable for differentiating said mammalian livestock undifferentiated stem cells into blood cells;
c. said conditions comprise culturing said cells in a culture medium suitable for differentiating said mammalian livestock undifferentiated stem cells into fat cells;
d. said conditions comprise culturing said cells in a culture medium suitable for differentiating said mammalian livestock undifferentiated stem cells into connective tissue cells;
e. said culturing in steps (a) and (b) is performed in a suspension culture;
g. said culturing in said suspension culture is without adherence to a substrate; and
h. any combination of (a) to (g).

53.-58. (canceled)

59. A method of preparing a food product, comprising combining differentiated mammalian livestock cells resultant from the method of claim 52 with a food product, thereby preparing the food product.

60. A food product comprising differentiated mammalian livestock cells resultant from the method of claim 52.
